(19) United States
(12) Patent Application Publication
(10) Pub. No.: US 2023/0123981 A1
(43) Pub. Date: Apr. 20, 2023

(54) 4'-O-METHYLENE PHOSPHONATE NUCLEIC ACIDS AND ANALOGUES THEREOF

(71) Applicant: Dicerna Pharmaceuticals, Inc., Lexington, MA (US)

(72) Inventors: Weimin WANG, Cambridge, MA (US); Hongchuan YU, Chestnut Hill, MA (US)

(21) Appl. No.: 17/792,948

(22) PCT Filed: Jan. 15, 2021
(86) PCT No.: PCT/US2021/013525

§ 371 Date: Jul. 14, 2022

Related U.S. Application Data
(60) Provisional application No. 62/961,360, filed on Jan. 15, 2020.
(60) Provisional application No. 62/975,352, filed on Feb. 12, 2020.
(60) Provisional application No. 62/991,738, filed on Mar. 19, 2020.

(51) Int. Cl. C12N 15/113

(52) U.S. Cl. CPC C12N 15/113; C12N 2310/3125; C12N 2310/14

(57) ABSTRACT

The present invention relates to nucleic acids and analogues thereof useful as potent and stable RNA interference agents.

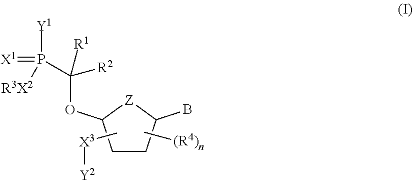

### CROSS-REFERENCE TO RELATED APPLICATIONS

This application claims the benefit under 35 U.S.C. § 119(e) of U.S. Provisional Patent Application No. 62/961,360, filed Jan. 15, 2020; U.S. Provisional Patent Application No. 62/975,352, filed Feb. 12, 2020; and U.S. Provisional Patent Application No. 62/991,738, filed Mar. 19, 2020, the contents of each of which are herein incorporated by reference in their entireties.

### TECHNICAL FIELD OF THE INVENTION

The present disclosure relates to nucleic acids and analogues thereof, and methods useful to modulate the expression of a target gene in a cell using the provided nucleic acids and analogues thereof according to the description provided herein. The disclosure also provides pharmaceutically acceptable compositions comprising the nucleic acids and analogues thereof of the present description and methods of using said compositions in the treatment of various disorders.

### BACKGROUND OF THE INVENTION

Regulation of gene expression by modified nucleic acids shows great potential as both a research tool in the laboratory and a therapeutic approach in the clinic. Several classes of oligonucleotide or nucleic acid-based therapeutics have been under the clinical investigation, including antisense oligo (ASO), short interfering RNA (siRNA), aptamer, ribozyme, exon skipping or splice altering oligos, mRNA, and CRISPR. Chemical modifications play a key role in overcoming the hurdles facing oligonucleotide therapeutics, including improving nuclease stability, RNA-binding affinity, and pharmacokinetic properties of oligonucleotides. Various chemical modification strategies for oligonucleotides have been developed in the past three decades including modification of the sugars, nucleobases, and phosphodiester backbone (Deleavey and Darma, CHEM. BIOL. 2012, 19(8):937-54; Wan and Seth, J. MED. CHEM. 2016, 59(21):9645-67; and Egli and Manoharan, ACC. CHEM. RES. 2019, 54(4):1036-47).

One of the most widely used backbone modifications in ASO and siRNA therapeutics is the phosphorothioate (PS) linkage, which replaces one of the non-bridging oxygen with a sulfur atom. Although this modification increases nuclease resistance and improves pharmacokinetics of therapeutic oligonucleotides without compromising their biological function, toxicities such as inflammation, nephrotoxicity, hepatotoxicity, and thrombocytopenia in both pre-clinical models and the clinic are known (Frazier, TOXICOL. PATHOL. 2015, 43(1):78-89). Toxicity is believed to arise from the ASO's strong tendency of binding to protein via the PS linkages (Shen et al, NAT. BIOTECH. 2019, 37:640-50). Furthermore, the PS linkages are chiral, resulting in 2^{N} diastereomers with N being the number of PS linkages in the backbone. Despite decades of efforts (Stec et al, NUCLEIC ACID RES. 1991, 1(21):5883-8 and J. AM. CHEM. SOC. 1998, 120(29):7156-67; Agrawal et al, TETRAHEDRON 1995, 6(5):1051-4; Iyer et al, J. AM. CHEM. SOC. 2000, 112(3), 1253-4; and Oka et al, J. AM. CHEM. SOC. 2008, 130(47):16031-7) including recent developments (Iwamoto et al, NAT. BIOTECH. 2017, 35(9):845-51) in the chemical synthesis of oligonucleotides with defined stereochemistry of PS linkages, the methods still lack of high stereoselectivity and high synthesis efficiency, and they are not generally robust and accessible. It is desirable to develop novel internucleotide linkages that not only can maintain the desired properties of PS linkages such as nuclease resistance, RNA-binding affinity, and proper pharmacokinetics, but also can mitigate toxicity without compromising the biological function. Ideally, the novel linkages should be achiral. Even if chirality cannot be avoided, controlling the stereochemistry should be robust and easily accessible.

Recently, charge-neutral alkyl phosphonate linkages have been reported and used to replace PS linkages in ASOs for reducing toxicity and increasing the therapeutic window (Migawa et al, NUCLEIC ACIDS RES. 2019, 47(11):5465-79 and Shen et al, 2019). However, these alkyl phosphonate linkages are chiral, do not support the RNase H mediated activity near the site of incorporation, and are more susceptible to strand cleavage under the basic conditions required to deprotect oligonucleotides after solid-phase synthesis.

An ongoing need exists in the art for effective treatments for disease, especially cancer. Nucleic acid therapeutic agents that are useful to modulate the expression of a target gene in a cell hold promise as therapeutic agents. Accordingly, there remains a need to find nucleic acids and analogues thereof that are useful as therapeutic agents.

### SUMMARY

The present application relates to novel nucleic acids or analogues thereof comprising 4′-O-methylene phosphonate internucleotide linkages, which function to modulate the expression of a target gene in a cell, and methods of preparation and uses thereof. The nucleic acids and analogues thereof provided herein are stable and bind to RNA targets to elicit RNase H activity comparable to their phosphorothioate (PS) counterparts and are also useful in splice switching and RNAi. The provided nucleic acids and analogues thereof can also be used in other mechanisms such as splice switching, RNAi, etc. Incorporation of the 4′-O-methylene phosphonate linkage confers nuclease stability to the internucleotide linkages, does not create a chiral center at the phosphorus atom, and retains the negative charge of the phosphate backbone which may be required for protein (e.g. RNase H or Ago2) binding to exert potent gene silencing activity in contrast to charge-neutral alkyl phosphonate approaches (Migawa et al, 2019).

Suitable nucleic acids or analogues thereof comprising 4′-O-methylene phosphonate internucleotide linkages include nucleic acid inhibitor molecules, such as dsRNAi inhibitor molecules, antisense oligonucleotides, miRNA, ribozymes, antagomirs, aptamers, and ssRNAi inhibitor molecules. In particular, the present disclosure provides nucleic acids and analogues thereof, which find utility as modulators of intracellular RNA levels, which are then reduced by the nucleic acids and analogues thereof as described herein. Nucleic acid inhibitor molecules can modulate RNA expression through a diverse set of mechanisms, for example by RNA interference (RNAi). An advantage of the nucleic acids and analogues thereof provided herein is that a broad range of pharmacological activities is possible, consistent with the modulation of intracellular RNA levels. In addition, the description provides methods of using an effective amount of the nucleic acids and analogues thereof as described herein for the treatment or amelioration of a disease condition, such as a cancer, viral infection or genetic disorder.

It has now been found that the nucleic acids and analogues thereof of this invention, and pharmaceutically acceptable compositions thereof, are effective as modulators of intracellular RNA levels. Such nucleic acids and analogues thereof comprise a 4′-O-methylene phosphonate internucleotide linkage, wherein the 4′-O-methylene phosphonate internucleotide linkage is represented by formula I:

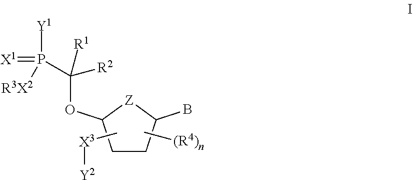

or a pharmaceutically acceptable salt thereof, wherein each variable is as defined and described herein.

Nucleic acids and analogues thereof of the present disclosure, and pharmaceutically acceptable compositions thereof, are useful for treating a variety of diseases, disorders or conditions, associated with regulation of intracellular RNA levels. Such diseases, disorders, or conditions include those described herein.

Nucleic acids and analogues thereof provided by this disclosure are also useful for the study of gene expression in biological and pathological phenomena; the study of RNA levels in bodily tissues; and the comparative evaluation of new RNA interference agents, in vitro or in vivo.

### BRIEF DESCRIPTION OF THE DRAWINGS

FIG. 1 includes the results of replacing internucleotide phosphorothioate (PS) linkage on benchmark SGLT2 ASO with internucleotide phosphodiester (PO) linkage showing % SGLT2 remaining compared to PBS (y-axis) and PBS, benchmark SGLT2 ASO (ASO), and oligonucleotide replaced between nucleotide 1 and 2 (ASO1), 2 and 3 (ASO2), 3 and 4 (ASO3), 4 and 5 (ASO4), 5 and 6 (ASO5), 6 and 7 (ASO6), 7 and 8 (ASO7), 8 and 9 (ASO8), 9 and 10 (ASO9), 10 and 11 (ASO10), and 11 and 12 (ASO11), counting from 5′-end to 3′-end respectively (x-axis).

FIG. 2 includes the results of replacing internucleotide phosphorothioate (PS) linkage with internucleotide 4′-O-methylene phosphonate (iMOP) linkage on the ASO backbone in vivo as measured by SGLT2 mRNA knockdown (KD) in mouse kidney 5 days after a single dose of 0.5 and 3.0 milligram per kilogram body weight (mpk) (% Expression [Slc5a2/Ppib]+SEM)) (y-axis) of PBS, SGLT2 benchmark ASO (ASO), ASO12, and ASO13 (x-axis). ASO12 is an experimental control only differing from the benchmark by the 2′-modification of the nucleotide 11 (counting from 5′-end) being 2′-OMe instead of 2′-MOE. ASO13 is a test article of which the linkage between nucleotide 10 and 11 is iMOP (shown in nucleic acid I-3) instead of PS. The rest of ASO12 is identical to ASO13.

FIG. 3 includes the results of the effect of replacing PS linkage with internucleotide 4′-O-methylene phosphonate (iMOP) linkage or internucleotide 4′-O-methylmethylene phosphonate (iMeMOP) linkage on ASO backbone in vivo as measured by SGLT2 mRNA knockdown (KD) in mouse kidney 7 days after a single dose of 0.5 milligram per kilogram body weight (mpk) showing (% SGLT2 mRNA remaining relative to PBS) (y-axis) and ASO14, SGLT2 benchmark ASO (ASO), ASO12, ASO13, and ASO15 (x-axis). ASO14 is a PO control of which the linkage between nucleotide 10 and 11 is a phosphodiester linkage and nucleotide 11 is 2′-OMe. ASO12 is a PS control of which all linkages are PS and nucleotide 11 is 2′-OMe. ASO13 is the iMOP test article of which the linkage between nucleotide 10 and 11 is iMOP instead of PS. ASO15 is the iMeMOP test article of which the linkage between nucleotide 10 and 11 is iMeMOP (shown in nucleic acid I-6) instead of PS.

FIG. 4 includes the results of iMOP linkage at 5′-end of antisense strand in a GalXC molecule as measured by target gene mRNA knockdown in mouse liver 4 days after a single dose of 1.0 mpk showing (% Aldh2 mRNA remaining relative to PBS) (y-axis) and PBS, GalXC1, and GalXC2 (x-axis). GalXC1 is a control GalXC molecule with a PS linkage between nucleotide 1 and 2 at the 5′-end of the antisense strand. GalXC2 is a GalXC molecule replacing the 5′-end PS linkage of the antisense strand with an iMOP linkage. The rest of the GalXC molecules are identical to the control.

FIG. 5 discloses effect of replacing PS linkage with iMOP linkage or iMeMOP linkage on the GAP2 position of the ASO backbone in vivo.

FIG. 6 depicts the results of the HRMS based in vitro tritosomal stability assay for benchmark ASO (A), ASO12 (B), ASO13 (C), ASO14 (D), and ASO15 (E) showing percent remaining (%) (y-axis) over tritosomal incubation time (hrs) (x-axis), as described in Table 3 of Example 8.

FIG. 7 includes the thermal stability results of incorporating iMeMOP and iMOP into the ASO strand of an ASO:RNA duplex for benchmark ASO:RNA1, ASO12:RNA1, ASO13:RNA1, ASO15:RNA1, and ASO14:RNA1 showing normalized absorbance (y-axis) over temperature (° C.) (x-axis).

FIG. 8 includes the RNase H activity results of incorporating iMeMOP and iMOP into the ASO strand of an ASO:RNA hybrid for benchmark ASO:RNA2, ASO15:RNA2, and ASO13:RNA2 showing percent remaining RNA (%)(y-axis) over time (min)(x-axis).

### DETAILED DESCRIPTION OF CERTAIN EMBODIMENTS

### 1. General Description of Certain Embodiments of the Invention

4′-O-Methylene phosphonate chemistry for the 5′-terminal phosphate mimic that improves RNAi potency and duration has been described in WO 2018/045317 and U.S. 2019/177729, the entirety of which is herein incorporated by reference. This type of chemical analogue not only mimics the electrostatic and/or steric properties of a phosphate group, but also possesses excellent metabolic stability, and is fully compatible with the standard oligonucleotide solid-phase synthesis.

Nucleic acids and analogues thereof of the present disclosure, and compositions thereof, are useful as RNA interference agents. In some embodiments, a provided nucleic acid or analogue thereof inhibits gene expression in a cell.

In certain embodiments, the present invention provides a nucleic acid or analogue thereof comprising a 4′-O-methylene phosphonate internucleotide linkage, wherein the 4′-O-methylene phosphonate internucleotide linkage is represented by formula I:

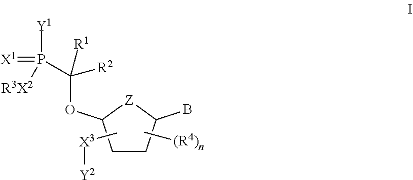

or a pharmaceutically acceptable salt thereof, wherein:
B is a nucleobase or hydrogen;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X¹ is O, S, or NR;
X² is —O—, —S—, —B(H)₂—, or a covalent bond;
X³ is —O—, —S—, —Se—, or —N(R)—;
Y¹ is a linking group attaching to the 2′- or 3′-terminal of a nucleoside, a nucleotide, or an oligonucleotide;
Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
Z is —O—, —S—, —N(R)—, or —C(R)₂—; and
n is 0, 1, 2, 3, 4, or 5.

### 2. Compounds and Definitions

Compounds of the present invention (i.e., nucleic acids and analogues thereof) include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally, or alternatively, any substitutable nitrogen of a bridged bicyclic group is optionally substituted. Exemplary bridged bicyclics include:

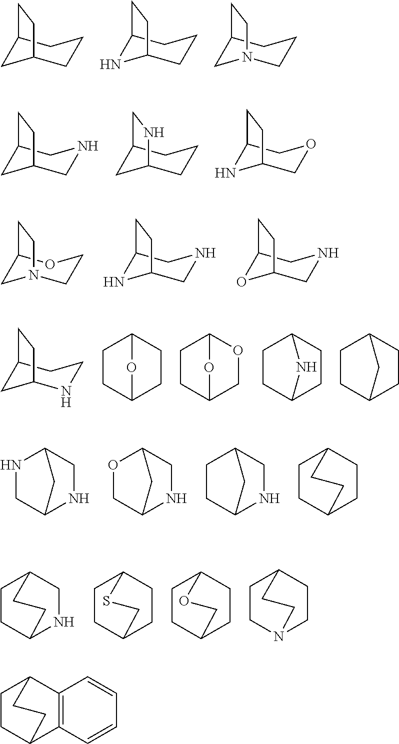

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

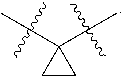 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the singular forms “a,” “an,” and “the” include plural references unless the context clearly dictates otherwise. For example, a reference to “a method” includes one or more methods, and/or steps of the type described herein and/or which will become apparent to those persons skilled in the art upon reading this disclosure and so forth.

As used herein, the term “and/or” is used in this disclosure to mean either “and” or “or” unless indicated otherwise.

As used herein, the term “4′-O-methylene phosphonate” refers all substituted methylene analogues (e.g., methylene substituted with methyl, dimethyl, ethyl, fluoro, cyclopropyl, etc.) and all phosphonate analogues (e.g., phosphorothioate, phosphorodithiolate, phosphodiester etc.) described herein.

As used herein, the term “5′-terminal nucleotide” refers to the nucleotide located at the 5′-end of an oligonucleotide. The 5′-terminal nucleotide may also be referred to as the “N1 nucleotide” in this application.

As used herein, the term “aptamer” refers to an oligonucleotide that has binding affinity for a specific target including a nucleic acid, a protein, a specific whole cell or a particular tissue. Aptamers may be obtained using methods known in the art, for example, by in vitro selection from a large random sequence pool of nucleic acids. Lee et al., NUCLEIC ACID RES., 2004, 32:D95-D100.

As used herein, the term “antagomir” refers to an oligonucleotide that has binding affinity for a specific target including the guide strand of an exogenous RNAi inhibitor molecule or natural miRNA (Krutzfeldt et al. NATURE 2005, 438(7068):685-689).

A double stranded RNAi inhibitor molecule comprises two oligonucleotide strands: an antisense strand and a sense strand. The antisense strand or a region thereof is partially, substantially or fully complementary to a corresponding region of a target nucleic acid. In addition, the antisense strand of the double stranded RNAi inhibitor molecule or a region thereof is partially, substantially or fully complementary to the sense strand of the double stranded RNAi inhibitor molecule or a region thereof. In certain embodiments, the antisense strand may also contain nucleotides that are non-complementary to the target nucleic acid sequence. The non-complementary nucleotides may be on either side of the complementary sequence or may be on both sides of the complementary sequence. In certain embodiments, where the antisense strand or a region thereof is partially or substantially complementary to the sense strand or a region thereof, the non-complementary nucleotides may be located between one or more regions of complementarity (e.g., one or more mismatches). The antisense strand of a double stranded RNAi inhibitor molecule is also referred to as the guide strand.

As used herein, the term “canonical RNA inhibitor molecule” refers to two strands of nucleic acids, each 21 nucleotides long with a central region of complementarity that is 19 base-pairs long for the formation of a double stranded nucleic acid and two nucleotide overhands at each of the 3′-ends.

As used herein, the term “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. “Fully complementarity” or 100% complementarity refers to the situation in which each nucleotide monomer of a first oligonucleotide strand or of a segment of a first oligonucleotide strand can form a base pair with each nucleotide monomer of a second oligonucleotide strand or of a segment of a second oligonucleotide strand. Less than 100% complementarity refers to the situation in which some, but not all, nucleotide monomers of two oligonucleotide strands (or two segments of two oligonucleotide strands) can form base pairs with each other. “Substantial complementarity” refers to two oligonucleotide strands (or segments of two oligonucleotide strands) exhibiting 90% or greater complementarity to each other. “Sufficiently complementary” refers to complementarity between a target mRNA and a nucleic acid inhibitor molecule, such that there is a reduction in the amount of protein encoded by a target mRNA.

As used herein, the term “complementary strand” refers to a strand of a double stranded nucleic acid inhibitor molecule that is partially, substantially or fully complementary to the other strand.

As used herein, the term “conventional antisense oligonucleotide” refers to single stranded oligonucleotides that inhibit the expression of a targeted gene by one of the following mechanisms: (1) Steric hindrance, e.g., the antisense oligonucleotide interferes with some step in the sequence of events involved in gene expression and/or production of the encoded protein by directly interfering with, for example, transcription of the gene, splicing of the pre-mRNA and translation of the mRNA; (2) Induction of enzymatic digestion of the RNA transcripts of the targeted gene by RNase H; (3) Induction of enzymatic digestion of the RNA transcripts of the targeted gene by RNase L; (4) Induction of enzymatic digestion of the RNA transcripts of the targeted gene by RNase P: (5) Induction of enzymatic digestion of the RNA transcripts of the targeted gene by double stranded RNase; and (6) Combined steric hindrance and induction of enzymatic digestion activity in the same antisense oligo. Conventional antisense oligonucleotides do not have an RNAi mechanism of action like RNAi inhibitor molecules. RNAi inhibitor molecules can be distinguished from conventional antisense oligonucleotides in several ways including the requirement for Ago2 that combines with an RNAi antisense strand such that the antisense strand directs the Ago2 protein to the intended target(s) and where Ago2 is required for silencing of the target.

Clustered Regularly Interspaced Short Palindromic Repeats (“CRISPR”) is a microbial nuclease system involved in defense against invading phages and plasmids. Wright et al., Cell, 2016, 164:29-44. This prokaryotic system has been adapted for use in editing target nucleic acid sequences of interest in the genome of eukaryotic cells. Cong et al., SCIENCE, 2013, 339:819-23; Mali et al., SCIENCE, 2013, 339:823-26; Woo Cho et al., NAT. BIOTECHNOLOGY, 2013, 31(3):230-232. As used herein, the term “CRISPR RNA” refers to a nucleic acid comprising a “CRISPR” RNA (crRNA) portion and/or a trans activating crRNA (tracrRNA) portion, wherein the CRISPR portion has a first sequence that is partially, substantially or fully complementary to a target nucleic acid and a second sequence (also called the tracer mate sequence) that is sufficiently complementary to the tracrRNA portion, such that the tracer mate sequence and tracrRNA portion hybridize to form a guide RNA. The guide RNA forms a complex with an endonuclease, such as a Cas endonuclease (e.g., Cas9) and directs the nuclease to mediate cleavage of the target nucleic acid. In certain embodiments, the crRNA portion is fused to the tracrRNA portion to form a chimeric guide RNA. Jinek et al., SCIENCE, 2012, 337:816-21. In certain embodiments, the first sequence of the crRNA portion includes between about 16 to about 24 nucleotides, preferably about 20 nucleotides, which hybridize to the target nucleic acid. In certain embodiments, the guide RNA is about 10-500 nucleotides. In other embodiments, the guide RNA is about 20-100 nucleotides.

As used herein, the term “delivery agent” refers to a transfection agent or a ligand that is complexed with or bound to an oligonucleotide and which mediates its entry into cells. The term encompasses cationic liposomes, for example, which have a net positive charge that binds to the oligonucleotide's negative charge. This term also encompasses the conjugates as described herein, such as GalNAc and cholesterol, which can be covalently attached to an oligonucleotide to direct delivery to certain tissues. Further specific suitable delivery agents are also described herein.

As used herein, the term “deoxyribonucleotide” refers to a nucleotide which has a hydrogen group at the 2′-position of the sugar moiety.

As used herein, the term “disulfide” refers to a chemical compound containing the group

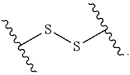 [SMILES: *SS*]

Typically, each sulfur atom is covalently bound to a hydrocarbon group. In certain embodiments, at least one sulfur atom is covalently bound to a group other than a hydrocarbon. The linkage is also called an SS-bond or a disulfide bridge.

As used herein, the term “duplex” in reference to nucleic acids (e.g., oligonucleotides), refers to a double helical structure formed through complementary base pairing of two antiparallel sequences of nucleotides.

As used herein, the term “excipient” refers to a non-therapeutic agent that may be included in a composition, for example to provide or contribute to a desired consistency or stabilizing effect.

As used herein, the term “furanose” refers to a carbohydrate having a five-membered ring structure, where the ring structure has 4 carbon atoms and one oxygen atom represented by

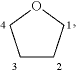

wherein the numbers represent the positions of the 4 carbon atoms in the five-membered ring structure.

As used herein, the term “glutathione” (GSH) refers to a tripeptide having structure

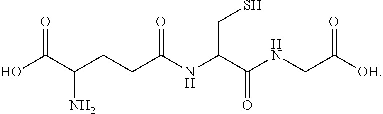 [SMILES: NC(CCC(=O)NC(CS)C(=O)NCC(=O)O)C(=O)O]

GSH is present in cells at a concentration of approximately 1-10 mM. GSH reduces glutathione-sensitive bonds, including disulfide bonds. In the process, glutathione is converted to its oxidized form, glutathione disulfide (GSSG). Once oxidized, glutathione can be reduced back by glutathione reductase, using NADPH as an electron donor.

As used herein, the terms “glutathione-sensitive compound”, or “glutathione-sensitive moiety”, are used interchangeably and refers to any chemical compound (e.g., oligonucleotide, nucleotide, or nucleoside) or moiety containing at least one glutathione-sensitive bond, such as a disulfide bridge or a sulfonyl group. As used herein, a “glutathione-sensitive oligonucleotide” is an oligonucleotide containing at least one nucleotide containing a glutathione-sensitive bond. A glutathione-sensitive moiety can be located at the 2′-carbon or 3′-carbon of the sugar moiety and comprises a sulfonyl group or a disulfide bridge. In certain embodiment, a glutathione-sensitive moiety is compatible with phosphoramidite oligonucleotide synthesis methods, as described, for example, in International Patent Application No. PCT/US2017/048239, which is hereby incorporated by reference in its entirety. A glutathione-sensitive moiety can also be located at the phosphorous containing internucleotide linkage. In certain embodiment, a glutathione-sensitive moiety is selected from those as described in PCT/US2013/072536, which is hereby incorporated by reference in its entirety.

As used herein, the term “internucleotide linking group” or “internucleotide linkage” refers to a chemical group capable of covalently linking two nucleoside moieties. Typically, the chemical group is a phosphorus-containing linkage group containing a phospho or phosphite group. Phospho linking groups are meant to include a phosphodiester linkage, a phosphorodithioate linkage, a phosphorothioate linkage, a phosphotriester linkage, a thionoalkylphosphonate linkage, a thionalkylphosphotriester linkage, a phosphoramidite linkage, a phosphonate linkage and/or a boranophosphate linkage. Many phosphorus-containing linkages are well known in the art, as disclosed, for example, in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050. In other embodiments, the oligonucleotide contains one or more internucleotide linking groups that do not contain a phosphorous atom, such short chain alkyl or cycloalkyl internucleotide linkages, mixed heteroatom and alkyl or cycloalkyl internucleotide linkages, or one or more short chain heteroaromatic or heterocyclic internucleotide linkages, including, but not limited to, those having siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; and amide backbones. Non-phosphorous containing linkages are well known in the art, as disclosed, for example, in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

As used herein, the term “loop” refers to a structure formed by a single strand of a nucleic acid, in which complementary regions that flank a particular single stranded nucleotide region hybridize in a way that the single stranded nucleotide region between the complementary regions is excluded from duplex formation or Watson-Crick base pairing. A loop is a single stranded nucleotide region of any length. Examples of loops include the unpaired nucleotides present in such structures as hairpins and tetraloops.

As used herein, the terms “microRNA” “mature microRNA” “miRNA” and “miR” are interchangeable and refer to non-coding RNA molecules encoded in the genomes of plants and animals. Typically, mature microRNA are about 18-25 nucleotides in length. In certain instances, highly conserved, endogenously expressed microRNAs regulate the expression of genes by binding to the 3′-untranslated regions (3′-UTR) of specific mRNAs. Certain mature microRNAs appear to originate from long endogenous primary microRNA transcripts (also known as pre-microRNAs, pri-microRNAs, pri-mirs, pri-miRs or pri-pre-microRNAs) that are often hundreds of nucleotides in length (Lee, et al., EMBO 1, 2002, 21(17), 4663-4670).

As used herein, the term “modified nucleoside” refers to a nucleoside containing one or more of a modified or universal nucleobase or a modified sugar. The modified or universal nucleobases (also referred to herein as base analogs) are generally located at the 1′-position of a nucleoside sugar moiety and refer to nucleobases other than adenine, guanine, cytosine, thymine and uracil at the 1′-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). A modified sugar (also referred herein to a sugar analog) includes modified deoxyribose or ribose moieties, e.g., where the modification occurs at the 2′, 3′-, 4′, or 5′-carbon position of the sugar. The modified sugar may also include non-natural alternative carbon structures such as those present in locked nucleic acids (“LNA”) (see, e.g., Koshkin et al. (1998), TETRAHEDRON, 54, 3607-3630); bridged nucleic acids (“BNA”) (see, e.g., U.S. Pat. No. 7,427,672 and Mitsuoka et al. (2009), NUCLEIC ACIDS RES., 37(4):1225-38); and unlocked nucleic acids (“UNA”) (see, e.g., Snead et al. (2013), MOLECULAR THERAPY—NUCLEIC ACIDS, 2, e103 (doi:10.1038/mtna.2013.36)). Suitable modified or universal nucleobases or modified sugars in the context of the present disclosure are described herein.

As used herein, the term “modified nucleotide” refers to a nucleotide containing one or more of a modified or universal nucleobase, a modified sugar, or a modified phosphate. The modified or universal nucleobases (also referred to generally herein as nucleobase) are generally located at the 1′-position of a nucleoside sugar moiety and refer to nucleobases other than adenine, guanine, cytosine, thymine and uracil at the 1′-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). A modified sugar (also referred herein to a sugar analog) includes modified deoxyribose or ribose moieties, e.g., where the modification occurs at the 2′-, 3′-, 4′-, or 5′-carbon position of the sugar. The modified sugar may also include non-natural alternative carbon structures such as those present in locked nucleic acids (“LNA”) (see, e.g., Koshkin et al. (1998), TETRAHEDRON, 54, 3607-3630), bridged nucleic acids (“BNA”) (see, e.g., U.S. Pat. No. 7,427,672 and Mitsuoka et al. (2009), NUCLEIC ACIDS RES., 37(4):1225-38); and unlocked nucleic acids (“UNA”) (see, e.g., Snead et al. (2013), MOLECULAR THERAPY—NUCLEIC ACIDS, 2, e103(doi: 10.1038/mtna.2013.36)). Modified phosphate groups refer to a modification of the phosphate group that does not occur in natural nucleotides and includes non-naturally occurring phosphate mimics as described herein. Modified phosphate groups also include non-naturally occurring internucleotide linking groups, including both phosphorous containing internucleotide linking groups and non-phosphorous containing linking groups, as described herein. Suitable modified or universal nucleobases, modified sugars, or modified phosphates in the context of the present disclosure are described herein.

As used herein, the term “naked nucleic acid” refers to a nucleic acid that is not formulated in a protective lipid nanoparticle or other protective formulation and is thus exposed to the blood and endosomal/lysosomal compartments when administered in vivo.

As used herein, the term “natural nucleoside” refers to a heterocyclic nitrogenous base in N-glycosidic linkage with a sugar (e.g., deoxyribose or ribose or analog thereof). The natural heterocyclic nitrogenous bases include adenine, guanine, cytosine, uracil and thymine.

As used herein, the term “natural nucleotide” refers to a heterocyclic nitrogenous base in N-glycosidic linkage with a sugar (e.g., ribose or deoxyribose or analog thereof) that is linked to a phosphate group. The natural heterocyclic nitrogenous bases include adenine, guanine, cytosine, uracil and thymine.

As used herein, the term “nucleic acid or analogue thereof” refers to any natural or modified nucleotide, nucleoside, oligonucleotide, conventional antisense oligonucleotide, ribonucleotide, deoxyribonucleotide, ribozyme, RNAi inhibitor molecule, antisense oligo (ASO), short interfering RNA (siRNA), canonical RNA inhibitor molecule, aptamer, antagomir, exon skipping or splice altering oligos, mRNA, miRNA, or CRISPR nuclease systems comprising one or more of the 4′-O-methylene phosphonate internucleotide linkage described herein. In certain embodiments, the provided nucleic acids or analogues thereof are used in antisense oligonucleotides, siRNA, and dicer substrate siRNA, including those described in U.S. 2010/331389, U.S. Pat. Nos. 8,513,207, 10,131,912, 8,927,705, CA 2,738,625, EP 2,379,083, and EP 3,234,132, the entirety of each of which is herein incorporated by reference.

As used herein, the term “nucleic acid inhibitor molecule” refers to an oligonucleotide molecule that reduces or eliminates the expression of a target gene wherein the oligonucleotide molecule contains a region that specifically targets a sequence in the target gene mRNA. Typically, the targeting region of the nucleic acid inhibitor molecule comprises a sequence that is sufficiently complementary to a sequence on the target gene mRNA to direct the effect of the nucleic acid inhibitor molecule to the specified target gene. The nucleic acid inhibitor molecule may include ribonucleotides, deoxyribonucleotides, and/or modified nucleotides.

As used herein, the term “nucleobase” refers to a natural nucleobase, a modified nucleobase, or a universal nucleobase. The nucleobase is the heterocyclic moiety which is located at the 1′ position of a nucleotide sugar moiety in a modified nucleotide that can be incorporated into a nucleic acid duplex (or the equivalent position in a nucleotide sugar moiety substitution that can be incorporated into a nucleic acid duplex). Accordingly, the present invention provides a nucleic acid and analogue thereof comprising a 4′-O-methylene phosphonate internucleotide linkage, wherein the 4′-O-methylene phosphonate internucleotide linkage is represented by formula I where the nucleobase is generally either a purine or pyrimidine base. In some embodiments, the nucleobase can also include the common bases guanine (G), cytosine (C), adenine (A), thymine (T), or uracil (U), or derivatives thereof, such as protected derivatives suitable for use in the preparation of oligonucleotides. In some embodiments, each of nucleobases G, A, and C independently comprises a protecting group selected from isobutyryl, acetyl, difluoroacetyl, trifluoroacetyl, phenoxyacetyl, isopropylphenoxyacetyl, benzoyl, 9-fluorenylmethoxycarbonyl, phenoxyacetyl, dimethylformamidine, dibutylforamidine and N,N-diphenylcarbamate. Nucleobase analogs can duplex with other bases or base analogs in dsRNAs. Nucleobase analogs include those useful in the nucleic acids and analogues thereof and methods of the invention, e.g., those disclosed in U.S. Pat. Nos. 5,432,272 and 6,001,983 to Benner and U.S. Patent Publication No. 20080213891 to Manoharan, which are herein incorporated by reference. Non-limiting examples of nucleobases include hypoxanthine (I), xanthine (X), 3β-D-ribofuranosyl-(2,6-diaminopyrimidine) (K), 3-O-D-ribofuranosyl-(1-methyl-pyrazolo[4,3-d]pyrimidine-5,7(4H,6H)-dione) (P), iso-cytosine (iso-C), iso-guanine (iso-G), 1-β-D-ribofuranosyl-(5-nitroindole), 1-β-D-ribofuranosyl-(3-nitropyrrole), 5-bromouracil, 2-aminopurine, 4-thio-dT, 7-(2-thienyl)-imidazo[4,5-b]pyridine (Ds) and pyrrole-2-carbaldehyde (Pa), 2-amino-6-(2-thienyl)purine (S), 2-oxopyridine (Y), difluorotolyl, 4-fluoro-6-methylbenzimidazole, 4-methylbenzimidazole, 3-methyl isocarbostyrilyl, 5-methyl isocarbostyrilyl, and 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl, and structural derivatives thereof (Schweitzer et al., J. ORG. CHEM., 59:7238-7242 (1994); Berger et al., NUCLEIC ACIDS RESEARCH, 28(15):2911-2914 (2000); Moran et al., J. AM. CHEM. SOC., 119:2056-2057 (1997); Morales et al., J. AM. CHEM. SOC., 121:2323-2324 (1999); Guckian et al., J. AM. CHEM. SOC., 118:8182-8183 (1996); Morales et al., J. AM. CHEM. SOC., 122(6):1001-1007 (2000); McMinn et al., J. AM. CHEM. SOC., 121:11585-11586 (1999); Guckian et al., J. ORG. CHEM., 63:9652-9656 (1998); Moran et al., PROC. NATL. ACAD. SCI., 94:10506-10511 (1997); Das et al., J. CHEM. SOC., PERKIN TRANS., 1:197-206 (2002); Shibata et al., J. CHEM. SOC., Perkin Trans., 1: 1605-1611 (2001); Wu et al., J. AM. CHEM. SOC., 122(32):7621-7632 (2000); O'Neill et al., J. ORG. CHEM., 67:5869-5875 (2002); Chaudhuri et al., J. AM. CHEM. SOC., 117:10434-10442 (1995); and U.S. Pat. No. 6,218,108). Base analogs may also be a universal base.

As used herein, the term “nucleoside” refers to a natural nucleoside or a modified nucleoside.

As used herein, the term “nucleotide” refers to a natural nucleotide or a modified nucleotide.

As used herein, the term “nucleotide position” refers to a position of a nucleotide in an oligonucleotide as counted from the nucleotide at the 5′-terminus. For example, nucleotide position 1 refers to the 5′-terminal nucleotide of an oligonucleotide.

As used herein, the term “oligonucleotide” as used herein refers to a polymeric form of nucleotides ranging from 2 to 2500 nucleotides. Oligonucleotides may be single-stranded or double-stranded. In certain embodiments, the oligonucleotide has 500-1500 nucleotides, typically, for example, where the oligonucleotide is used in gene therapy. In certain embodiments, the oligonucleotide is single or double stranded and has 7-100 nucleotides. In certain embodiments, the oligonucleotide is single or double stranded and has 15-100 nucleotides. In another embodiment, the oligonucleotide is single or double stranded has 15-50 nucleotides, typically, for example, where the oligonucleotide is a nucleic acid inhibitor molecule. In another embodiment, the oligonucleotide is single or double stranded has 25-40 nucleotides, typically, for example, where the oligonucleotide is a nucleic acid inhibitor molecule. In yet another embodiment, the oligonucleotide is single or double stranded and has 19-40 or 19-25 nucleotides, typically, for example, where the oligonucleotide is a double-stranded nucleic acid inhibitor molecule and forms a duplex of at least 18-25 base pairs. In other embodiments, the oligonucleotide is single stranded and has 15-25 nucleotides, typically, for example, where the oligonucleotide nucleotide is a single stranded RNAi inhibitor molecule. Typically, the oligonucleotide contains one or more phosphorous containing internucleotide linking groups, as described herein. In other embodiments, the internucleotide linking group is a non-phosphorus containing linkage, as described herein.

As used herein, the term “overhang” refers to terminal non-base pairing nucleotide(s) at either end of either strand of a double-stranded nucleic acid inhibitor molecule. In certain embodiments, the overhang results from one strand or region extending beyond the terminus of the complementary strand to which the first strand or region forms a duplex. One or both of two oligonucleotide regions that are capable of forming a duplex through hydrogen bonding of base pairs may have a 5′- and/or 3′-end that extends beyond the 3′- and/or 5′-end of complementarity shared by the two polynucleotides or regions. The single-stranded region extending beyond the 3′- and/or 5′-end of the duplex is referred to as an overhang.

As used herein, the term “pharmaceutical composition” comprises a pharmacologically effective amount of a phosphate analog-modified oligonucleotide and a pharmaceutically acceptable excipient. As used herein, “pharmacologically effective amount” “therapeutically effective amount” or “effective amount” refers to that amount of a phosphate analog-modified oligonucleotide of the present disclosure effective to produce the intended pharmacological, therapeutic or preventive result.

As used herein, the term “pharmaceutically acceptable excipient”, means that the excipient is suitable for use with humans and/or animals without undue adverse side effects (such as toxicity, irritation, and allergic response) commensurate with a reasonable benefit/risk ratio.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the nucleic acids and analogues thereof of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “suitable prodrug” is meant to indicate a compound that may be converted under physiological conditions or by solvolysis to a biologically active nucleic acid or analogue thereof described herein. Thus, the term “prodrug” refers to a precursor of a biologically active nucleic acid or analogue thereof that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., DESIGN OF PRODRUGS (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in BIOREVERSIBLE CARRIERS IN DRUG DESIGN, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of suitable prodrugs include, but are not limited to glutathione, acyloxy, thioacyloxy, 2-carboalkoxyethyl, disulfide, thiaminal, and enol ester derivatives of a phosphorus atom-modified nucleic acid. The term “pro-oligonucleotide” or “pronucleotide” or “nucleic acid prodrug” refers to an oligonucleotide which has been modified to be a prodrug of the oligonucleotide. Phosphonate and phosphate prodrugs can be found, for example, in Wiener et al., “Prodrugs or phosphonates and phosphates: crossing the membrane” TOP. CURR. CHEM. 2015, 360:115-160, the entirety of which is herein incorporated by reference.

As used herein, the term “phosphoramidite” refers to a nitrogen containing trivalent phosphorus derivative. Examples of suitable phosphoramidites are described herein.

As used herein, “potency” refers to the amount of an oligonucleotide or other drug that must be administered in vivo or in vitro to obtain a particular level of activity against an intended target in cells. For example, an oligonucleotide that suppresses the expression of its target by 90% in a subject at a dosage of 1 mg/kg has a greater potency than an oligonucleotide that suppresses the expression of its target by 90% in a subject at a dosage of 100 mg/kg.

As used herein, the term “protecting group” is used in the conventional chemical sense as a group which reversibly renders unreactive a functional group under certain conditions of a desired reaction. After the desired reaction, protecting groups may be removed to deprotect the protected functional group. All protecting groups should be removable under conditions which do not degrade a substantial proportion of the molecules being synthesized.

As used herein, the term “provided nucleic acid” refers to any genus, subgenus, and/or species set forth herein.

As used herein, the term “ribonucleotide” refers to a natural or modified nucleotide which has a hydroxyl group at the 2′-position of the sugar moiety.

As used herein, the term “ribozyme” refers to a catalytic nucleic acid molecule that specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

As used herein, the term “RNAi inhibitor molecule” refers to either (a) a double stranded nucleic acid inhibitor molecule (“dsRNAi inhibitor molecule”) having a sense strand (passenger) and antisense strand (guide), where the antisense strand or part of the antisense strand is used by the Argonaute 2 (Ago2) endonuclease in the cleavage of a target mRNA or (b) a single stranded nucleic acid inhibitor molecule (“ssRNAi inhibitor molecule”) having a single antisense strand, where that antisense strand (or part of that antisense strand) is used by the Ago2 endonuclease in the cleavage of a target mRNA.

A double stranded RNAi inhibitor molecule comprises two oligonucleotide strands: an antisense strand and a sense strand. The sense strand or a region thereof is partially, substantially or fully complementary to the antisense strand of the double stranded RNAi inhibitor molecule or a region thereof. In certain embodiments, the sense strand may also contain nucleotides that are non-complementary to the antisense strand. The non-complementary nucleotides may be on either side of the complementary sequence or may be on both sides of the complementary sequence. In certain embodiments, where the sense strand or a region thereof is partially or substantially complementary to the antisense strand or a region thereof, the non-complementary nucleotides may be located between one or more regions of complementarity (e.g., one or more mismatches). The sense strand is also called the passenger strand.

As used herein, the term “systemic administration” refers to in vivo systemic absorption or accumulation of drugs in the blood stream followed by distribution throughout the entire body.

As used herein, the term “target site” “target sequence,” “target nucleic acid”, “target region,” “target gene” are used interchangeably and refer to a RNA or DNA sequence that is “targeted,” e.g., for cleavage mediated by an RNAi inhibitor molecule that contains a sequence within its guide/antisense region that is partially, substantially, or perfectly or sufficiently complementary to that target sequence.

As used herein, the term “tetraloop” refers to a loop (a single stranded region) that forms a stable secondary structure that contributes to the stability of an adjacent Watson-Crick hybridized nucleotides. Without being limited to theory, a tetraloop may stabilize an adjacent Watson-Crick base pair by stacking interactions. In addition, interactions among the nucleotides in a tetraloop include but are not limited to non-Watson-Crick base pairing, stacking interactions, hydrogen bonding, and contact interactions (Cheong et al., NATURE 1990; 346(6285):680-2; Heus and Pardi, SCIENCE 1991; 253(5016):191-4). A tetraloop confers an increase in the melting temperature (Tm) of an adjacent duplex that is higher than expected from a simple model loop sequence consisting of random bases. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. A tetraloop may contain ribonucleotides, deoxyribonucleotides, modified nucleotides, and combinations thereof. In certain embodiments, a tetraloop consists of four nucleotides. In certain embodiments, a tetraloop consists of five nucleotides.

Examples of RNA tetraloops include the UNCG family of tetraloops (e.g., UUCG), the GNRA family of tetraloops (e.g., GAAA), and the CUUG tetraloop. (Woese et al., PNAS, 1990, 87(21):8467-71; Antao et al., NUCLEIC ACIDS RES., 1991, 19(21):5901-5). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). (Nakano et al., BIOCHEMISTRY, 2002, 41(48):14281-14292. Shinji et al., NIPPON KAGAKKAI KOEN YOKOSHU, 2000, 78(2):731).

As used herein, “universal base” refers to a heterocyclic moiety located at the 1′ position of a nucleotide sugar moiety in a modified nucleotide, or the equivalent position in a nucleotide sugar moiety substitution, that, when present in a nucleic acid duplex, can be positioned opposite more than one type of base without altering the double helical structure (e.g., the structure of the phosphate backbone). Additionally, the universal base does not destroy the ability of the single stranded nucleic acid in which it resides to duplex to a target nucleic acid. The ability of a single stranded nucleic acid containing a universal base to duplex a target nucleic can be assayed by methods apparent to one in the art (e.g., UV absorbance, circular dichroism, gel shift, single stranded nuclease sensitivity, etc.). Additionally, conditions under which duplex formation is observed may be varied to determine duplex stability or formation, e.g., temperature, as melting temperature (Tm) correlates with the stability of nucleic acid duplexes. Compared to a reference single stranded nucleic acid that is exactly complementary to a target nucleic acid, the single stranded nucleic acid containing a universal base forms a duplex with the target nucleic acid that has a lower Tm than a duplex formed with the complementary nucleic acid. However, compared to a reference single stranded nucleic acid in which the universal base has been replaced with a base to generate a single mismatch, the single stranded nucleic acid containing the universal base forms a duplex with the target nucleic acid that has a higher Tm than a duplex formed with the nucleic acid having the mismatched base.

Some universal bases are capable of base pairing by forming hydrogen bonds between the universal base and all of the bases guanine (G), cytosine (C), adenine (A), thymine (T), and uracil (U) under base pair forming conditions. A universal base is not a base that forms a base pair with only one single complementary base. In a duplex, a universal base may form no hydrogen bonds, one hydrogen bond, or more than one hydrogen bond with each of G, C, A, T, and U opposite to it on the opposite strand of a duplex. Preferably, the universal bases do not interact with the base opposite to it on the opposite strand of a duplex. In a duplex, base pairing between a universal base occurs without altering the double helical structure of the phosphate backbone. A universal base may also interact with bases in adjacent nucleotides on the same nucleic acid strand by stacking interactions. Such stacking interactions stabilize the duplex, especially in situations where the universal base does not form any hydrogen bonds with the base positioned opposite to it on the opposite strand of the duplex. Non-limiting examples of universal-binding nucleotides include inosine, 1-O-D-ribo furanosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside, NUCLEIC ACIDS RES. 1995 Nov. 11; 23(21):4363-70; Loakes et al., 3-Nitropyrrole and 5-nitroindole as universal bases in primers for DNA sequencing and PCR, NUCLEIC ACIDS RES. 1995 Jul. 11; 23(13):2361-6; Loakes and Brown, 5-Nitroindole as a universal base analogue, NUCLEIC ACIDS RES. 1994 Oct. 11; 22(20):4039-43).

### 3. Description of Exemplary Embodiments

As described above, in certain embodiments, the present invention provides a nucleic acid or analogue thereof comprising a 4′-O-methylene phosphonate internucleotide linkage, wherein the 4′-O-methylene phosphonate internucleotide linkage is represented by formula I:

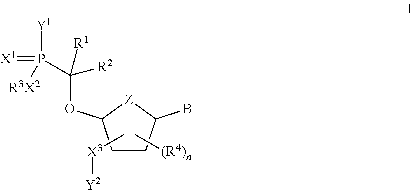

or a pharmaceutically acceptable salt thereof, wherein:
B is a nucleobase or hydrogen;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X¹ is O, S, or NR;
X² is —O—, —S—, —B(H)₂—, or a covalent bond;
X³ is —O—, —S—, —Se—, or —N(R)—;
Y¹ is a linking group attaching to the 2′- or 3′-terminal of a nucleoside, a nucleotide, or an oligonucleotide;
Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
Z is —O—, —S—, —N(R)—, or —C(R)₂—; and
n is 0, 1, 2, 3, 4, or 5.

As defined above and described herein, B is a nucleobase or hydrogen.

In some embodiments, B is a nucleobase. In some embodiments, B is a nucleobase analogue. In some embodiments, B is a modified nucleobase. In some embodiments, B is a universal nucleobase. In some embodiments, B is a hydrogen.

In some embodiments, B is selected from

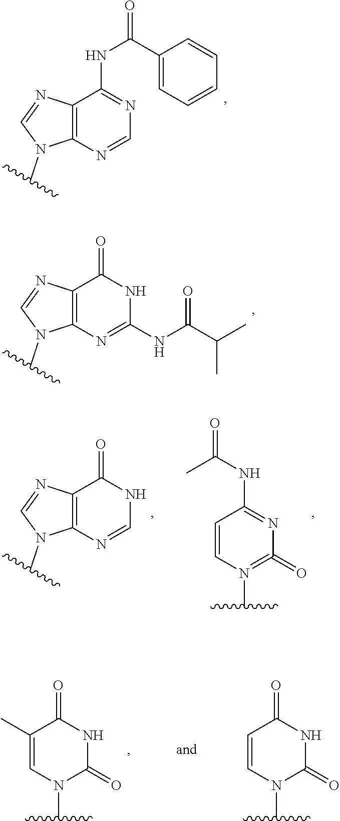

In some embodiments, B is selected from those depicted in Table 1.

As defined above and described herein, R¹ and R² are independently hydrogen, halogen, R³, —CN, —S(O)R, —S(O)₂R, —Si(OR)₂R, —Si(OR)R₂, or —SiR₃, or R¹ and R² on the same carbon are taken together with their intervening atoms to form a 3-7 membered saturated or partially unsaturated ring having 0-3 heteroatoms, independently selected from nitrogen, oxygen, and sulfur.

In some embodiments, R¹ and R² are independently hydrogen. In some embodiments, R¹ and R² are independently deuterium. In some embodiments, R¹ and R² are independently halogen. In some embodiments, R¹ and R² are independently R⁵. In some embodiments, R¹ and R² are independently —CN. In some embodiments, R¹ and R² are independently —S(O)R. In some embodiments, R¹ and R² are independently —S(O)₂R. In some embodiments, R¹ and R² are independently —Si(OR)₂R. In some embodiments, R¹ and R² are independently —Si(OR)R₂. In some embodiments, R¹ and R² are independently —SiR₃. In some embodiments, R¹ and R² on the same carbon are taken together with their intervening atoms to form a 3-7 membered saturated or partially unsaturated ring having 0-3 heteroatoms, independently selected from nitrogen, oxygen, and sulfur.

In some embodiments, R¹ is methyl and R² is hydrogen.

In some embodiments, R¹ and R² are selected from those depicted in Table 1.

As defined above and described herein, each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur.

In some embodiments, R is hydrogen. In some embodiments, R is a suitable protecting group. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is optionally substituted 5-6 membered heteroaryl ring having 1-4 heteroatoms independently selected from nitrogen, oxygen, and sulfur. In some embodiments, two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur.

In some embodiments, R is selected from those depicted in Table 1, below.

As defined above and described herein, R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R³ is hydrogen. In some embodiments, R³ is a suitable protecting group. In some embodiments, R³ is a suitable prodrug. In some embodiments, R³ is a suitable phosphate/phosphonate prodrug, which is a glutathione-sensitive moiety. In some embodiments, R³ is a glutathione-sensitive moiety selected from those as described in International Patent Application No. PCT/US2017/048239, which is hereby incorporated by reference in its entirety. In some embodiments, R³ is an optionally substituted C₁₋₆ aliphatic. In some embodiments, R³ is an optionally substituted phenyl. In some embodiments, R³ is an optionally substituted 4-7 membered saturated or partially unsaturated heterocyclic having 1-2 heteroatoms independently selected from nitrogen, oxygen, and sulfur. In some embodiments, R³ is optionally substituted 5-6 membered heteroaryl ring having 1-4 heteroatoms independently selected from nitrogen, oxygen, and sulfur.

In some embodiments, R³ is methyl. In some embodiments, R³ is ethyl. In some embodiments, R³ is

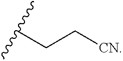 [SMILES: *CCC#N]

In some embodiments, R³ is

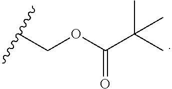 [SMILES: *COC(=O)C(C)(C)C]

In some embodiments, R³ is

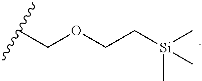 [SMILES: *COCC[Si](C)(C)C]

In some embodiments, R³ is selected from those depicted in Table 1, below.

As defined above and described herein, each R⁴ is independently hydrogen, a suitable prodrug, R⁵, halogen, —CN, —NO₂, —OR, —SR, —NR₂, —Si(OR)₂R, —Si(OR)R₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃.

In some embodiments, R⁴ is hydrogen. In some embodiments, R⁴ is deuterium. In some embodiments, R⁴ is a suitable prodrug. In some embodiments, R⁴ is a suitable phosphate/phosphonate prodrug, which is a glutathione-sensitive moiety. In some embodiments, R⁴ is a glutathione-sensitive moiety selected from those as described in International Patent Application No. PCT/US2013/072536, which is hereby incorporated by reference in its entirety. In some embodiments, R⁴ is R⁵. In some embodiments, R⁴ is halogen. In some embodiments, R⁴ is —CN. In some embodiments, R⁴ is —NO₂. In some embodiments, R⁴ is —OR. In some embodiments, R⁴ is —SR. In some embodiments, R⁴ is —NR₂. In some embodiments, R⁴ is —S(O)₂R. In some embodiments, R⁴ is —S(O)₂NR₂. In some embodiments, R⁴ is —S(O)R. In some embodiments, R⁴ is —C(O)R. In some embodiments, R⁴ is —C(O)OR. In some embodiments, R⁴ is —C(O)NR₂. In some embodiments, R⁴ is —C(O)N(R)OR. In some embodiments, R⁴ is —C(R)₂N(R)C(O)R. In some embodiments, R⁴ is —C(R)₂N(R)C(O)NR₂. In some embodiments, R⁴ is —OC(O)R. In some embodiments, R⁴ is —OC(O)NR₂. In some embodiments, R⁴ is —OP(O)R₂. In some embodiments, R⁴ is —OP(O)(OR)₂. In some embodiments, R⁴ is —OP(O)(OR)NR₂. In some embodiments, R⁴ is —OP(O)(NR₂)₂—. In some embodiments, R⁴ is —N(R)C(O)OR. In some embodiments, R⁴ is —N(R)C(O)R. In some embodiments, R⁴ is —N(R)C(O)NR₂. In some embodiments, R⁴ is —N(R)P(O)R₂. In some embodiments, R⁴ is —N(R)P(O)(OR)₂. In some embodiments, R⁴ is —N(R)P(O)(OR)NR₂. In some embodiments, R⁴ is —N(R)P(O)(NR₂)₂. In some embodiments, R⁴ is —N(R)S(O)₂R. In some embodiments, R⁴ is —Si(OR)₂R. In some embodiments, R⁴ is —Si(OR)R₂. In some embodiments, R⁴ is —SiR₃.

In some embodiments, R⁴ is hydroxyl. In some embodiments, R⁴ is fluoro. In some embodiments, R⁴ is methoxy. In some embodiments, R⁴ is

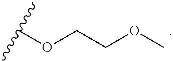 [SMILES: *OCCOC]

In some embodiments, R⁴ is selected from those depicted in Table 1.

As defined above and described herein, each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁵ is an optionally substituted C₁₋₆ aliphatic. In some embodiments, R⁵ is an optionally substituted phenyl. In some embodiments, R⁵ is an optionally substituted 4-7 membered saturated or partially unsaturated heterocyclic ring having 1-2 heteroatoms independently selected from nitrogen, oxygen, and sulfur. In some embodiments, R⁵ is an optionally substituted 5-6 membered heteroaryl ring having 1-4 heteroatoms independently selected from nitrogen, oxygen, and sulfur.

In some embodiments, R⁵ is selected from those depicted in Table 1, below.

As defined above and described herein, X¹ is O, S, or NR.

In some embodiments, X¹ is O. In some embodiments, X¹ is S. In some embodiments, X¹ is NR.

In some embodiments, X¹ is selected from those depicted in Table 1, below.

As defined above and described herein, X² is —O—, —S—, —B(H)₂—, or a covalent bond.

In some embodiments, X² is —O—. In some embodiments, X² is —S—. In some embodiments, X² is —B(H)₂—. In some embodiments, X² and R³ form —BH₃. In some embodiments, X² is a covalent bond. In some embodiments, X² is a covalent bond that constitutes a boranophosphate backbone.

In some embodiments, X² is selected from those depicted in Table 1, below.

As defined above and described herein, X³ is —O—, —S—, —Se—, or —N(R)—.

In some embodiments, X³ is —O—. In some embodiments, X³ is —S—. In some embodiments, X³ is —Se—. In some embodiments, X³ is —N(R)—.

In some embodiments, X³ is selected from those depicted in Table 1, below.

As defined above and described herein, Y¹ is a linking group attaching to the 2′- or 3′-terminal of a nucleoside, a nucleotide, or an oligonucleotide.

In some embodiments, Y¹ is a linking group attaching to the 2′-terminal of a nucleoside, a nucleotide, or an oligonucleotide. In some embodiments, Y¹ is a linking group attaching to the 3′-terminal of a nucleoside, a nucleotide, or an oligonucleotide.

In some embodiments, a linking group of Y¹ is a bond. In some embodiments, a linking group of Y¹ is a —C(R)₂—. In some embodiments, a linking group of Y¹ is a —CH₂—.

In some embodiments, Y¹ is

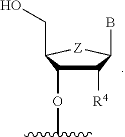

In some embodiments, Y¹ is

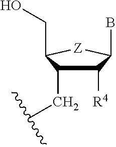

In some embodiments, Y¹ is

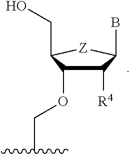

In some embodiments, Y¹ is

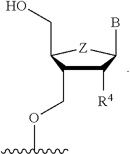

In some embodiments, Y¹ is

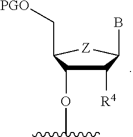

In some embodiments, Y¹ is

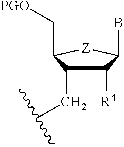

In some embodiments, Y¹ is

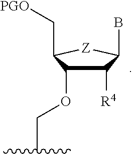

In some embodiments, Y¹ is

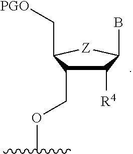

In some embodiments, Y¹ is

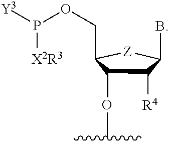

In some embodiments, Y¹ is

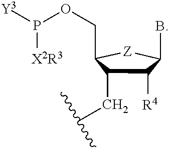

In some embodiments, Y¹ is

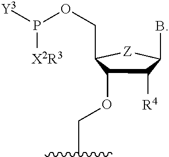

In some embodiments, Y¹ is

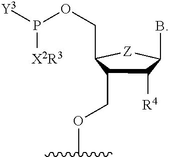

In some embodiments, Y¹ is

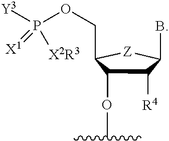

In some embodiments, Y¹ is

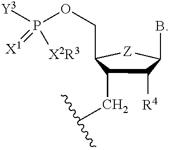

In some embodiments, Y¹ is

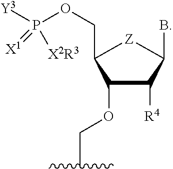

In some embodiments, Y¹ is

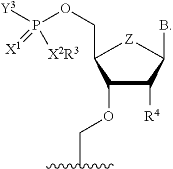

In some embodiments, Y¹ is selected from those depicted in Table 1, below.

As defined above and described herein, Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support.

In some embodiments, Y² is hydrogen. In some embodiments, Y² is a protecting group. In some embodiments, Y² is a phosphoramidite analogue. In some embodiments, Y² is a phosphoramidite analogue of formula:

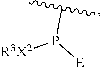

wherein each of R³, X², and E is independently as described herein. In some embodiments, Y² is an internucleotide linking group attaching to the 4′-terminal of a nucleoside, a nucleotide, or an oligonucleotide. In some embodiments, Y² is an internucleotide linking group attaching to the 5′-terminal of a nucleoside, a nucleotide, or an oligonucleotide. In some embodiments, Y² is a linking group attaching to a solid support.

In some embodiments, Y² is benzoyl. In some embodiments, Y² is t-butyldimethylsilyl. In some embodiments, Y² is

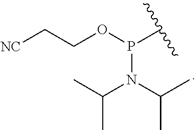 [SMILES: *P(OCCC#N)N(C(C)C)C(C)C]

In some embodiments, Y² is

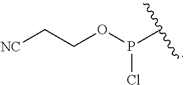 [SMILES: *P(Cl)OCCC#N]

In some embodiments, Y² is

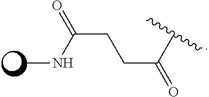 [SMILES: *C(=O)CCC(=O)NC]

In some embodiments, Y² is

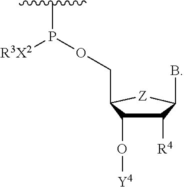

In some embodiments, Y² is

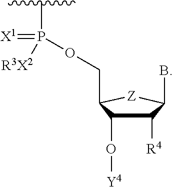

In some embodiments, Y² is selected from those depicted in Table 1, below.

As shown above in some embodiments of Y¹, Y³ is a linking group attaching to the 2′- or 3′-terminal of a nucleoside, a nucleotide, or an oligonucleotide.

In some embodiments, Y³ is a linking group attaching to the 2′-terminal of a nucleoside, a nucleotide, or an oligonucleotide. In some embodiments, Y³ is a linking group attaching to the 3′-terminal of a nucleoside, a nucleotide, or an oligonucleotide.

In some embodiments, Y³ is selected from those depicted in Table 1, below.

As shown above in some embodiments of Y², Y⁴ is hydrogen, a protecting group, a phosphoramidite analogue, an internucleotide linking group attaching to the 4′- or 5′-terminal of a nucleoside, a nucleotide, or an oligonucleotide, or a linking group attaching to a solid support.

In some embodiments, Y⁴ is hydrogen. In some embodiments, Y⁴ is a protecting group. In some embodiments, Y⁴ is a phosphoramidite analogue. In some embodiments, Y⁴ is a phosphoramidite analogue of formula:

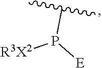

wherein each of R³, X², and E is independently as described herein. In some embodiments, Y⁴ is an internucleotide linking group attaching to the 4′-terminal of a nucleoside, a nucleotide, or an oligonucleotide. In some embodiments, Y⁴ is an internucleotide linking group attaching to the 5′-terminal of a nucleoside, a nucleotide, or an oligonucleotide. In some embodiments, Y⁴ is a linking group attaching to a solid support.

In some embodiments, Y⁴ is benzoyl. In some embodiments, Y⁴ is t-butyldimethylsilyl. In some embodiments, Y⁴ is

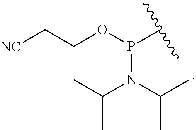 [SMILES: *P(OCCC#N)N(C(C)C)C(C)C]

In some embodiments, Y² is

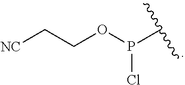 [SMILES: *P(Cl)OCCC#N]

In some embodiments, Y⁴ is

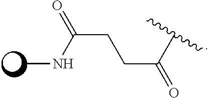 [SMILES: *C(=O)CCC(=O)NC]

In some embodiments, Y⁴ is selected from those depicted in Table 1, below.

As defined above and described herein, Z is —O—, —S—, —N(R)—, or —C(R)₂—.

In some embodiments, Z is —O—. In some embodiments, Z is —S—. In some embodiments, Z is —N(R)—. In some embodiments, Z is —C(R)₂—.

In some embodiments, Z is selected from those depicted in Table 1, below.

As defined above and described herein, n is 0, 1, 2, 3, 4, or 5.

In some embodiments, n is 0. In some embodiments, n is 1. In some embodiments, n is 2. In some embodiments, n is 3. In some embodiments, n is 4. In some embodiments, n is 5. In some embodiments, n is selected from those depicted in Table 1, below.

In some embodiments, a nucleic acid or analogue thereof comprising a 4′-O-methylene phosphonate internucleotide linkage does not comprise a methyl substitution at the 4′-C position. In some embodiments, the 4′-O-methylene phosphonate internucleotide linkage represented by formula I is not

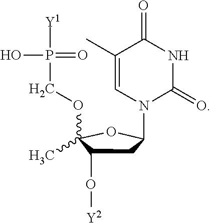

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, n is 1, and the connectivity and stereochemistry is as shown, thereby forming a nucleic acid or analogue thereof of formula I-a-1:

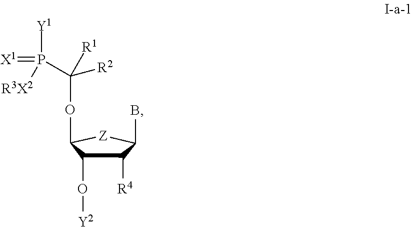

or a pharmaceutically acceptable salt thereof, wherein:
each of B, R¹, R², R³, R⁴, X¹, X², Y¹, Y², and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is a suitable hydroxyl protecting group (PG), n is 1, and the connectivity and stereochemistry is as shown, thereby forming a nucleic acid or analogue thereof of formula I-a-2:

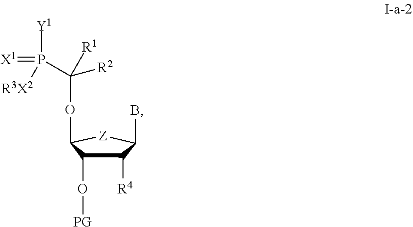

or a pharmaceutically acceptable salt thereof, wherein:
each of B, R¹, R², R³, R⁴, X¹, X², Y¹, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is hydrogen, n is 1, and the connectivity and stereochemistry is as shown, thereby forming a nucleic acid or analogue thereof of formula I-a-3:

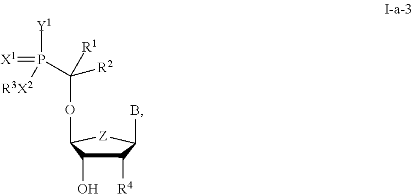

or a pharmaceutically acceptable salt thereof, wherein:
each of B, R¹, R², R³, R⁴, X¹, X², Y¹, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is phosphoramidite

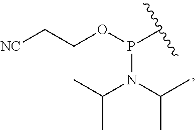 [SMILES: *P(OCCC#N)N(C(C)C)C(C)C]

n is 1, and the connectivity and stereochemistry is as shown, thereby forming formula a nucleic acid or analogue thereof of I-a-4:

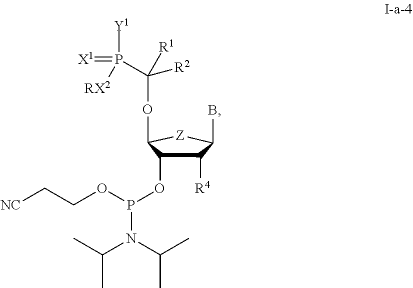

or a pharmaceutically acceptable salt thereof, wherein:
each of B, R¹, R², R³, R⁴, X¹, X², Y¹, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is linking group attaching to solid support

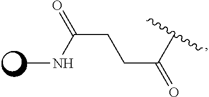 [SMILES: *C(=O)CCC(=O)NC]

n is 1, and the connectivity and stereochemistry is as shown, thereby forming a nucleic acid or analogue thereof of formula I-a-5:

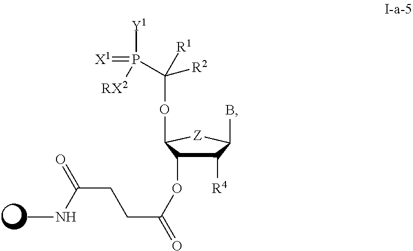

or a pharmaceutically acceptable salt thereof, wherein:
each of B, R¹, R², R³, R⁴, X¹, X², Y¹, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, n is 1, the connectivity and stereochemistry is as shown, and Y¹ is a covalent bond attaching to the 3′-hydroxyl of nucleoside

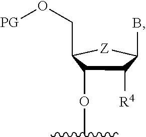

wherein PG of Y¹ is a suitable hydroxyl protection group, thereby forming a nucleic acid or analogue thereof of formula I-b-1:

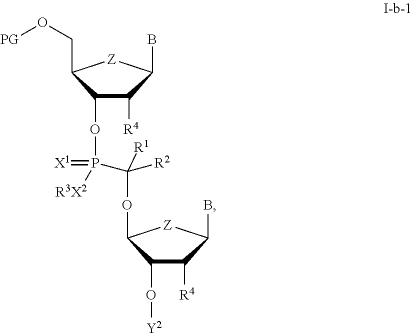

or a pharmaceutically acceptable salt thereof, wherein:
each of B, R¹, R², R³, R⁴, X¹, X², Y², and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is a suitable hydroxyl protecting group PG¹, n is 1, the connectivity and stereochemistry is as shown, and Y¹ is a covalent bond attaching to the 3′-hydroxyl of nucleoside

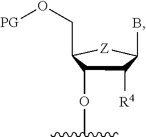

wherein PG of Y¹ is a suitable hydroxyl protection group, thereby forming a nucleic acid or analogue thereof of formula I-c-1:

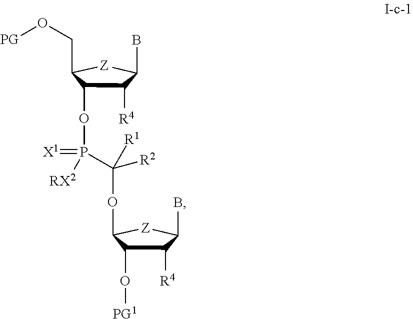

or a pharmaceutically acceptable salt thereof, wherein:
each of B, R¹, R², R³, R⁴, X¹, X², and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is hydrogen, n is 1, the connectivity and stereochemistry is as shown, and Y¹ is a covalent bond attaching to the 3′-hydroxyl of nucleoside

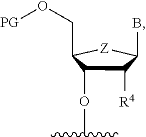

wherein PG of Y¹ is a suitable hydroxyl protection group, thereby a nucleic acid or analogue thereof of formula I-d-1:

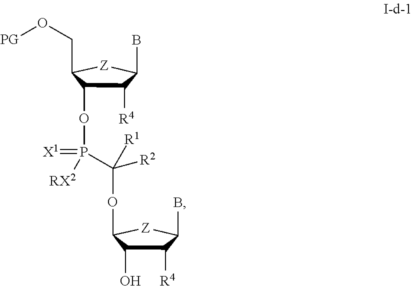

or a pharmaceutically acceptable salt thereof, wherein:
each of B, R¹, R², R³, R⁴, X¹, X², and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is phosphoramidite

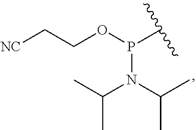 [SMILES: *P(OCCC#N)N(C(C)C)C(C)C]

n is 1, the connectivity and stereochemistry is as shown, and Y¹ is a covalent bond attaching to the 3′-hydroxyl of nucleoside

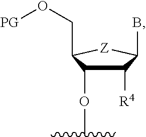

wherein PG of Y¹ is a suitable hydroxyl protection group, thereby forming a nucleic acid or analogue thereof of formula I-e-1:

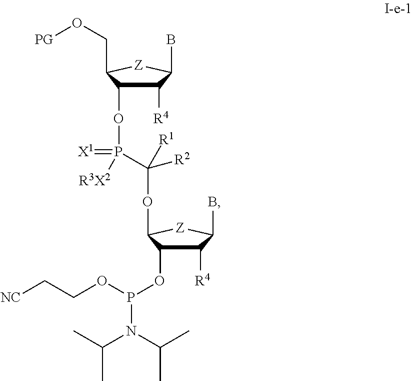

or a pharmaceutically acceptable salt thereof, wherein:
each of B, R¹, R², R³, R⁴, X¹, X², and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is linking group attaching to solid support

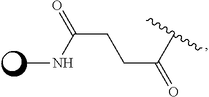 [SMILES: *C(=O)CCC(=O)NC]

n is 1, the connectivity and stereochemistry is as shown, and Y¹ is a covalent bond attaching to the 3′-hydroxyl of nucleoside

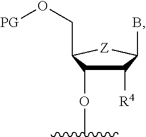

wherein PG of Y¹ is a suitable hydroxyl protection group, thereby forming a nucleic acid or analogue thereof of formula I-f-1:

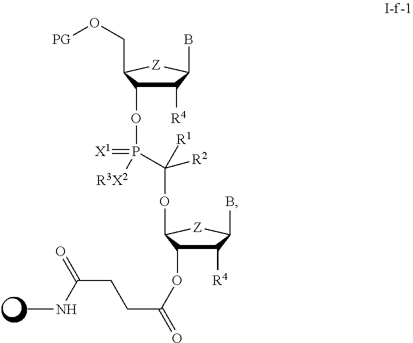

or a pharmaceutically acceptable salt thereof, wherein:
each of B, R¹, R², R³, R⁴, X¹, X², Y¹, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, n is 1, the connectivity and stereochemistry is as shown, and Y¹ is a covalent bond attaching to the 3′-hydroxyl of nucleoside

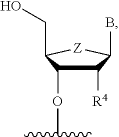

thereby forming a nucleic acid or analogue thereof of formula I-g-1:

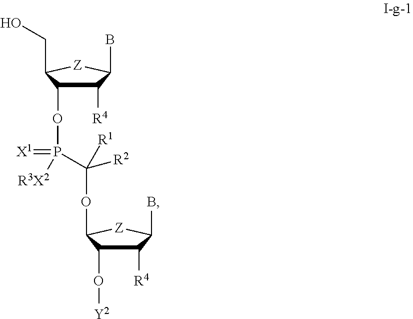

or a pharmaceutically acceptable salt thereof, wherein:
each of B, R¹, R², R³, R⁴, X¹, X², Y², and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, n is 1, the connectivity and stereochemistry is as shown, and Y¹ is a covalent bond attaching to the 3′-hydroxyl of oligonucleotide

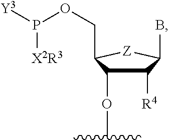

thereby forming a nucleic acid or analogue thereof of formula I-h-1:

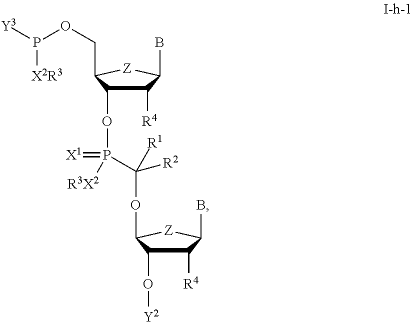

or a pharmaceutically acceptable salt thereof, wherein:
each of B, R¹, R², R³, R⁴, X¹, X², Y², Y³, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, n is 1, the connectivity and stereochemistry is as shown, and Y¹ is a covalent bond attaching to the 3′-hydroxyl of oligonucleotide

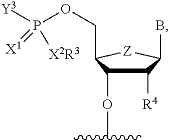

thereby forming an oligonucleotide of formula I-i-1:

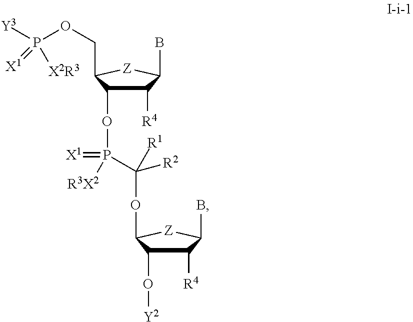

or a pharmaceutically acceptable salt thereof, wherein:
each of B, R¹, R², R³, R⁴, X¹, X², Y², Y³, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is a suitable hydroxyl protecting group PG¹, n is 1, the connectivity and stereochemistry is as shown, and Y¹ is a covalent bond attaching to the 3′-hydroxyl of oligonucleotide

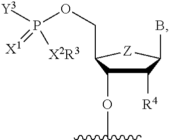

thereby forming an oligonucleotide of formula I-j-1:

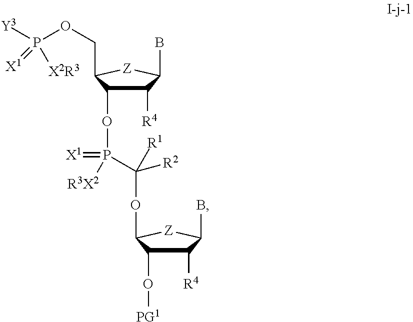

or a pharmaceutically acceptable salt thereof, wherein:
each of B, R¹, R², R³, R⁴, X¹, X², Y³, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is hydrogen, n is 1, the connectivity and stereochemistry is as shown, and Y¹ is a covalent bond attaching to the 3′-hydroxyl of oligonucleotide

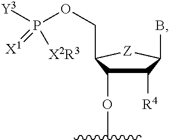

thereby forming an oligonucleotide of formula I-k-1:

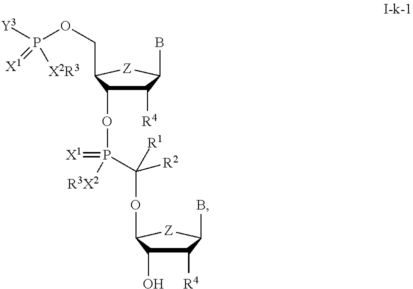

or a pharmaceutically acceptable salt thereof, wherein:
each of B, R¹, R², R³, R⁴, X¹, X², Y³, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is

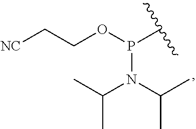 [SMILES: *P(OCCC#N)N(C(C)C)C(C)C]

n is 1, the connectivity and stereochemistry is as shown, and Y¹ is a covalent bond attaching to the 3′-hydroxyl of oligonucleotide

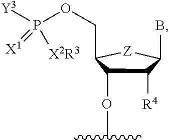

thereby forming an oligonucleotide of formula I-l-1:

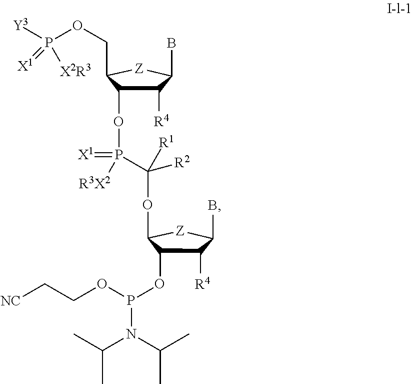

or a pharmaceutically acceptable salt thereof, wherein:
each of B, R¹, R², R³, R⁴, X¹, X², Y³, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, Y² is linking group attaching to solid support

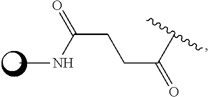 [SMILES: *C(=O)CCC(=O)NC]

n is 1, the connectivity and stereochemistry is as shown, and Y¹ is a covalent bond attaching to the 3′-hydroxyl of oligonucleotide

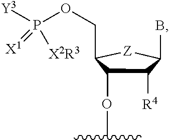

thereby forming an oligonucleotide of formula I-m-1:

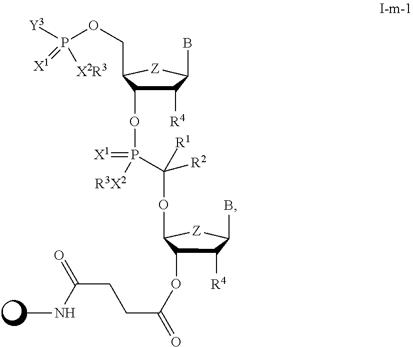

or a pharmaceutically acceptable salt thereof, wherein:
each of B, R¹, R², R³, R⁴, X¹, X², Y³, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, n is 1, the connectivity and stereochemistry is as shown, and Y¹ is a methylene group attaching to the 3′-hydroxyl of oligonucleotide

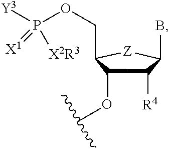

thereby forming an oligonucleotide of formula I-n-1:

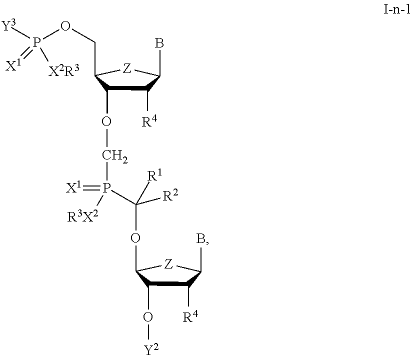

or a pharmaceutically acceptable salt thereof, wherein:
each of B, R¹, R², R³, R⁴, X¹, X², Y², Y³, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, n is 1, the connectivity and stereochemistry is as shown, and Y¹ is a methylene group attaching to the 3′-carbon of oligonucleotide

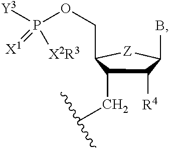

thereby forming an oligonucleotide of formula I-o-1:

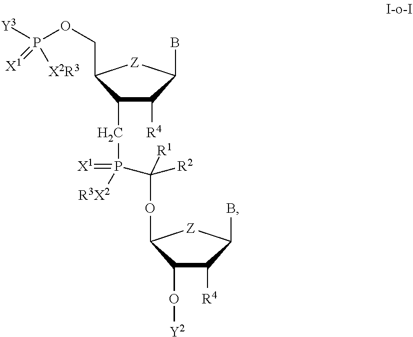

or a pharmaceutically acceptable salt thereof, wherein:
each of B, R¹, R², R³, R⁴, X¹, X², Y², Y³, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, n is 1, the connectivity and stereochemistry is as shown, Y¹ is a covalent bond attaching to the 3′-hydroxyl of nucleoside

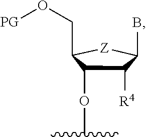

### and Y² is

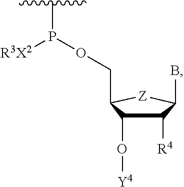

thereby forming an oligonucleotide of formula I-p-1:

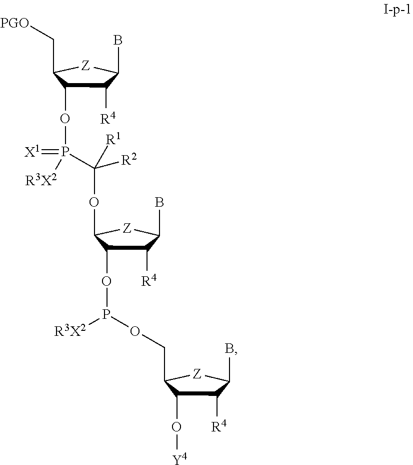

or a pharmaceutically acceptable salt thereof, wherein:
each of B, R¹, R², R³, R⁴, X¹, X², Y⁴, and Z is as defined above.

In some embodiments, the present invention provides a nucleic acid or analogue thereof comprising a 4′-O-methylene phosphonate internucleotide linkage, wherein the 4′-O-methylene phosphonate internucleotide linkage is represented by formula I wherein X³ is —O—, n is 1, the connectivity and stereochemistry is as shown, Y¹ is a covalent bond attaching to the 3′-hydroxyl of nucleoside

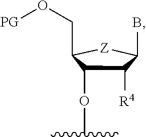

### and Y² is

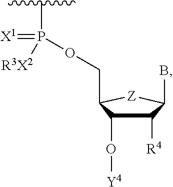

thereby forming an oligonucleotide of formula I-q-1:

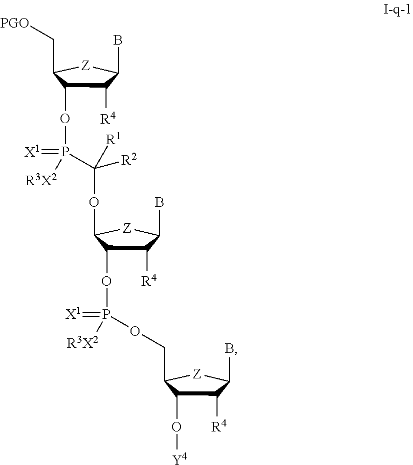

or a pharmaceutically acceptable salt thereof, wherein:
each of B, R¹, R², R³, R⁴, X¹, X², Y⁴, and Z is as defined above.

In certain embodiments, the present invention provides an oligonucleotide-ligand conjugate comprising an antisense strand of 15 to 30 nucleotides in length with one or more of any of the above disclosed nucleic acid analogues, and a sense strand of 10 to 53 nucleotides in length, in which the sense strand forms a duplex region with the antisense strand and the sense strand comprises one or more ligand moieties. In certain embodiments, the ligand moiety is a GalNAc.

In certain embodiments, the antisense strand comprises a 4′-O-methylene phosphonate internucleotide linkage at the 5′ end.

In certain embodiments, the present invention provides an oligonucleotide-ligand conjugate, or a pharmaceutically acceptable salt thereof, comprising:
a sense strand of 36 nucleotides in length, comprising 2′-fluoro modified nucleotides at positions 3, 5, 8, 10, 12, 13, 15, and 17, 2′-O-methyl modified nucleotides at positions 1, 2, 4, 6, 7, 9, 11, 14, 16, 18-27, and 31-36, and one phosphorothioate internucleotide linkage between the nucleotides at positions 1 and 2, wherein the nucleotides at positions 27-30 forms a tetraloop, and each of the nucleotides at positions 28-30 is conjugated to a monovalent GalNac moiety at the 2′ position; and
an antisense strand of 22 nucleotides in length, comprising 2′-fluoro modified nucleotides at positions 3, 4, 5, 7, 10, 14, 16, and 19, 2′-O-methyl modified nucleotides at positions 1, 2, 6, 8, 9, 11, 12, 13, 15, 17, 18, and 20-22, and three phosphorothioate internucleotide linkages between nucleotides at positions 2 and 3, between nucleotides at positions 20 and 21, and between nucleotides at positions 21 and 22, wherein the nucleotides at positions 1 and 2 form a 4′-O-methylene phosphonate internucleotide linkage having the following structure:

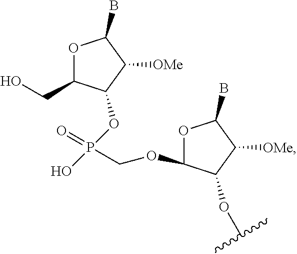

wherein each B is independently a nucleobase as described herein, for example, Adenine, Guanine, Cytosine, or Uracil.

In some embodiments, positions 27-30 of a sense strand forms a GAAA tetraloop.

In some embodiments, a nucleotide conjugated to a monovalent GalNac moiety at the 2′ position has the following structure:

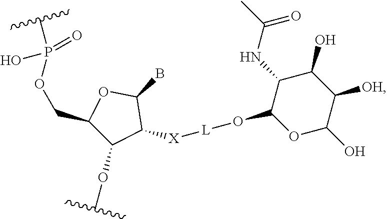

wherein B is a nucleobase as described herein, for example, Adenine, Guanine, Cytosine, or Uracil; X is a O, S, or N; and L is a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof. In some embodiments, L is an acetal linker. In some embodiments, X is O.

In some embodiments, a nucleotide conjugated to a monovalent GalNac moiety at the 2′ position has the following structure:

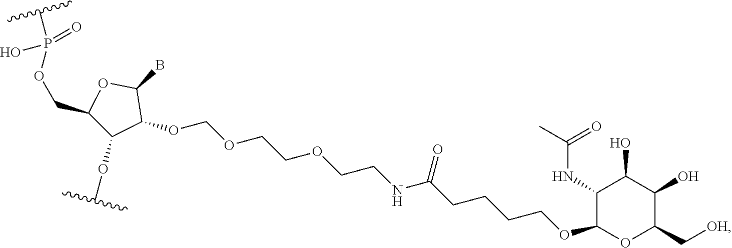

wherein B is a nucleobase as described herein, for example, Adenine, Guanine, Cytosine, or Uracil.

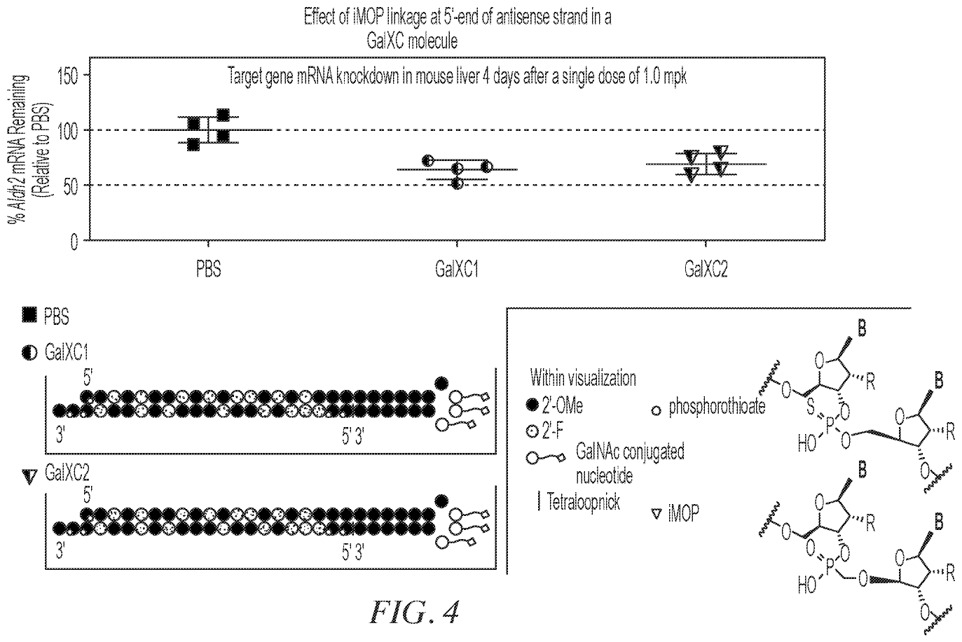

In some embodiments, the present invention provides an oligonucleotide-ligand conjugate having a structure of GalXC2 as shown in FIG. 4.

Exemplary nucleic acids and analogues thereof comprising a 4′-O-methylene phosphonate internucleotide linkage of the invention are set forth in Table 1 below.

TABLE 1
Exemplary Nucleic Acids and Analogues Thereof

| I-# | Structure |
|---|---|
| I-1 | 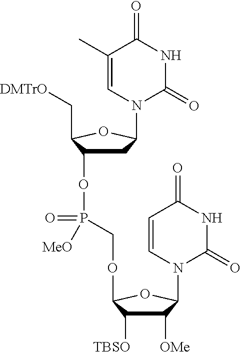 |
| I-2 | 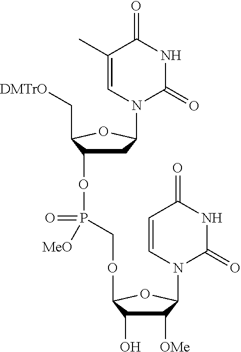 |
| I-3 | 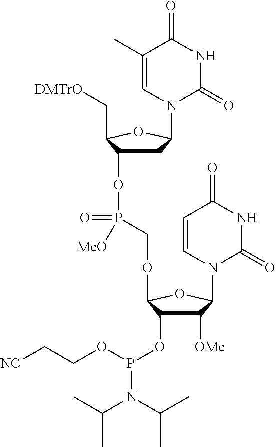 |
| I-4 | 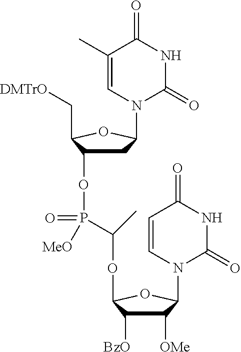 |
| I-5 | 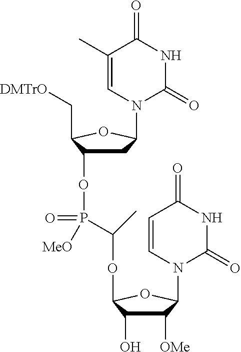 |
| I-6 | 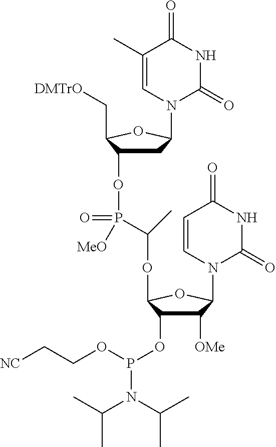 |
| I-7 | 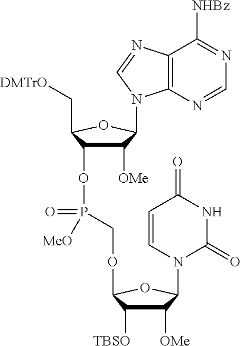 |
| I-8 | 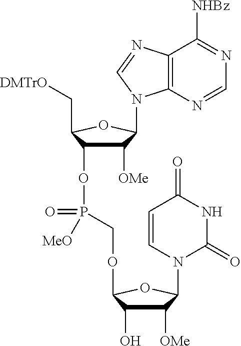 |
| I-9 | 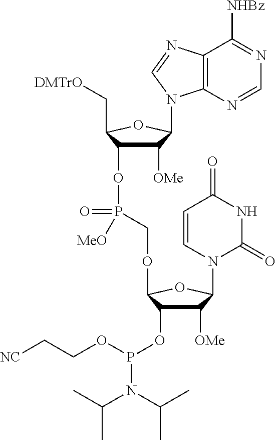 |
| I-10 | 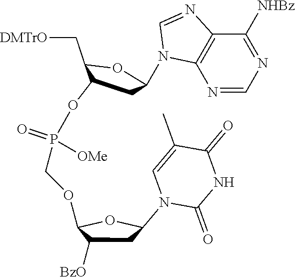 |
| I-11 | 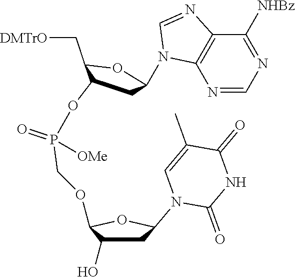 |
| I-12 | 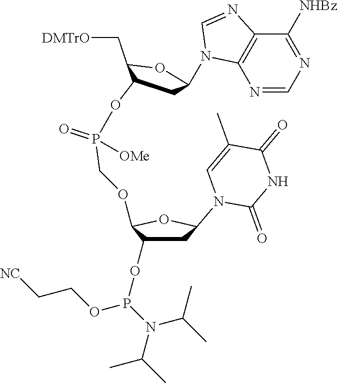 |
| I-13 | 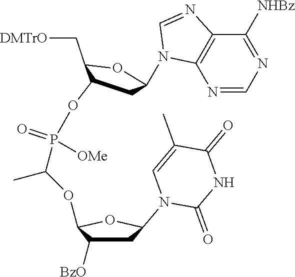 |
| I-14 | 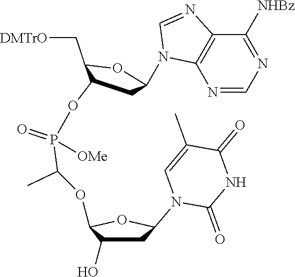 |
| I-15 | 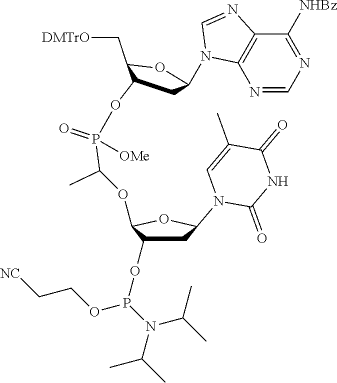 |
| I-16 | 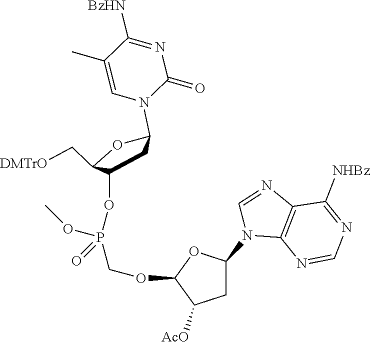 |
| I-17 | 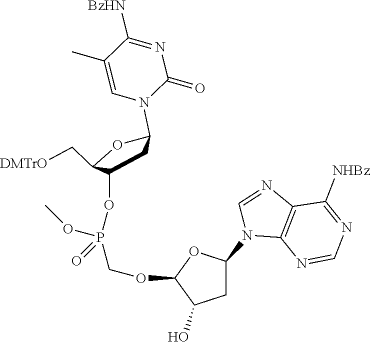 |
| I-18 | 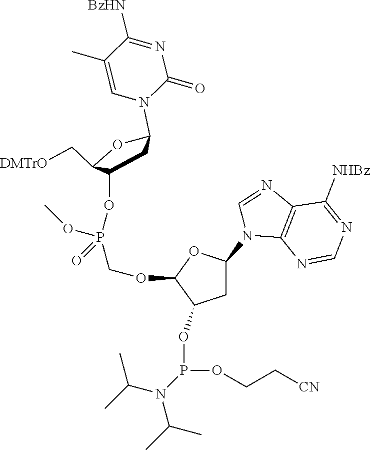 |

In some embodiments, the present invention provides a nucleic acid or analogue thereof comprising a 4′-O-methylene phosphonate internucleotide linkage of the invention set forth in Table 1, above, or a pharmaceutically acceptable salt thereof.

### 4. General Methods of Providing the Nucleic Acids and Analogues Thereof

The nucleic acids and analogues thereof comprising a 4′-O-methylene phosphonate internucleotide linkage described herein can be made using a variety of synthetic methods known in the art, including standard phosphoramidite methods. Any phosphoramidite synthesis method can be used to synthesize the provided nucleic acids of this invention. In certain embodiments, phosphoramidites are used in a solid phase synthesis method to yield reactive intermediate phosphite compounds, which are subsequently oxidized using known methods to produce phosphonate-modified oligonucleotides, typically with a phosphodiester or phosphorothioate internucleotide linkages. The oligonucleotide synthesis of the present disclosure can be performed in either direction: from 5′ to 3′ or from 3′ to 5′ using art known methods.

In certain embodiments, the method for synthesizing a provided nucleic acid comprises (a) attaching a nucleoside or analogue thereof to a solid support via a covalent linkage; (b) coupling a nucleoside phosphoramidite or analogue thereof to a reactive hydroxyl group on the nucleoside or analogue thereof of step (a) to form an internucleotide bond therebetween, wherein any uncoupled nucleoside or analogue thereof on the solid support is capped with a capping reagent; (c) oxidizing said internucleotide bond with an oxidizing agent; and (d) repeating steps (b) to (c) iteratively with subsequent nucleoside phosphoramidites or analogue thereof to form a nucleic acid or analogue thereof, wherein at least the nucleoside or analogue thereof of step (a), the nucleoside phosphoramidite or analogue thereof of step (b) or at least one of the subsequent nucleoside phosphoramidites or analogues thereof of step (d) comprises a phosphonate-containing moiety as described herein. Typically, the coupling, capping/oxidizing steps and optionally, deprotecting steps, are repeated until the oligonucleotide reaches the desired length and/or sequence, after which it is cleaved from the solid support.

In Scheme A below, where a particular protecting group, leaving group, or transformation condition is depicted, one of ordinary skill in the art will appreciate that other protecting groups, leaving groups, and transformation conditions are also suitable and are contemplated. Certain reactive functional groups (e.g., —N(H)—, —OH, etc.) envisioned in the genera in Scheme A requiring additional protection group strategies are also contemplated and is appreciated by those having ordinary skill in the art. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

In certain embodiments, nucleic acids and analogues thereof of the present invention are generally prepared according to Scheme A and Scheme B set forth below:

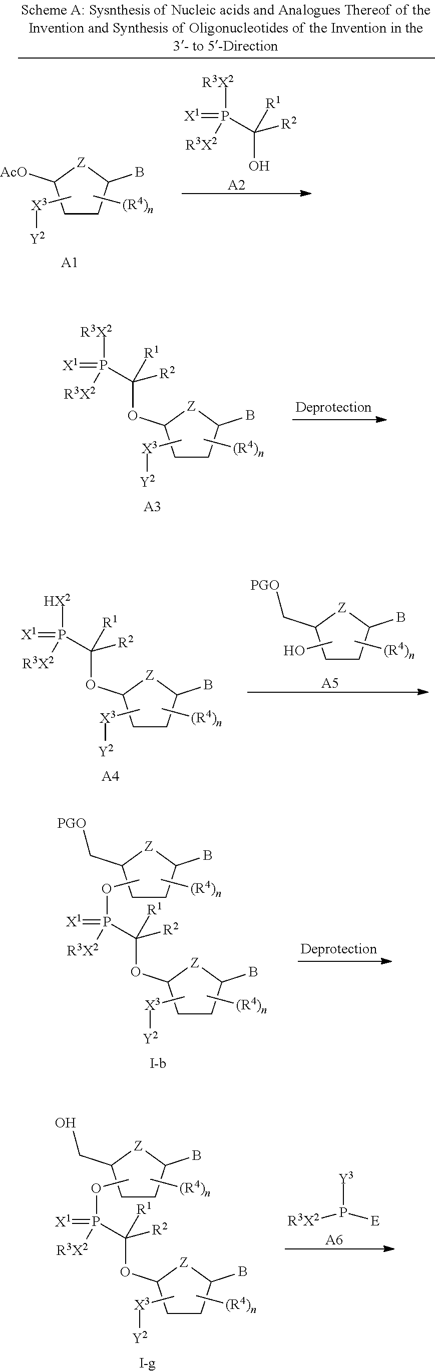

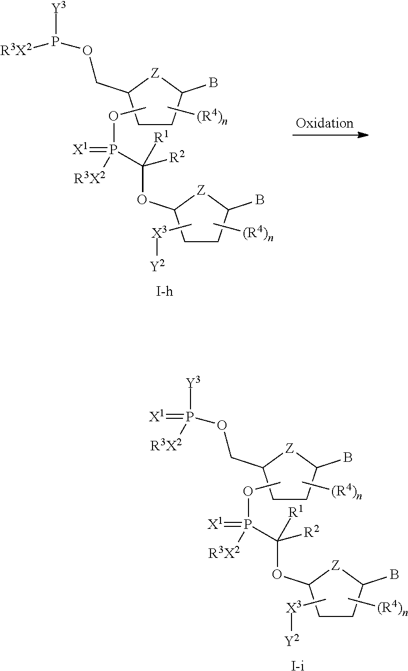

As depicted in Scheme A above, a nucleic acid or analogue thereof of formula A1 is coupled to a P(V) compound of formula A2 such as by using a Lewis acid (e.g., BF₃—OEt₂), to form a nucleic acid or analogue thereof of formula A3 comprising, but not limited to, 4′-O-methylene phosphonate. Nucleic acid or analogue thereof of formula A3 is then first deprotected (e.g., hydrolyzed) to form a nucleic acid or analogue thereof of formula A4 comprising, but not limited to, a hydrogen 4′-O-methylene phosphonate, followed by condensing with a nucleotide or analogue thereof of formula A5 to form nucleic acid or analogue thereof of formula I-b comprising, but not limited to, a 4′-O-methylene phosphonate internucleotide linkage of the invention. The nucleic acid or analogue thereof of formula I-b is then deprotected to form nucleic acid or analogue thereof of formula I-g and reacted with a phosphoramidite analogue of formula A6 to form a nucleic acid or analogue thereof of formula I-h comprising, but not limited to, a 4′-O-methylene phosphonate internucleotide linkage of the invention. Oxidation of nucleic acid or analogue thereof of formula I-h then affords an oligonucleotide compound of formula I-i comprising, but not limited to, a 4′-O-methylene phosphonate internucleotide linkage of the invention. Each of B, E, PG, R¹, R², R³, R⁴, X¹, X², X³, Y², Y³, Z, and n is as defined above and described herein.

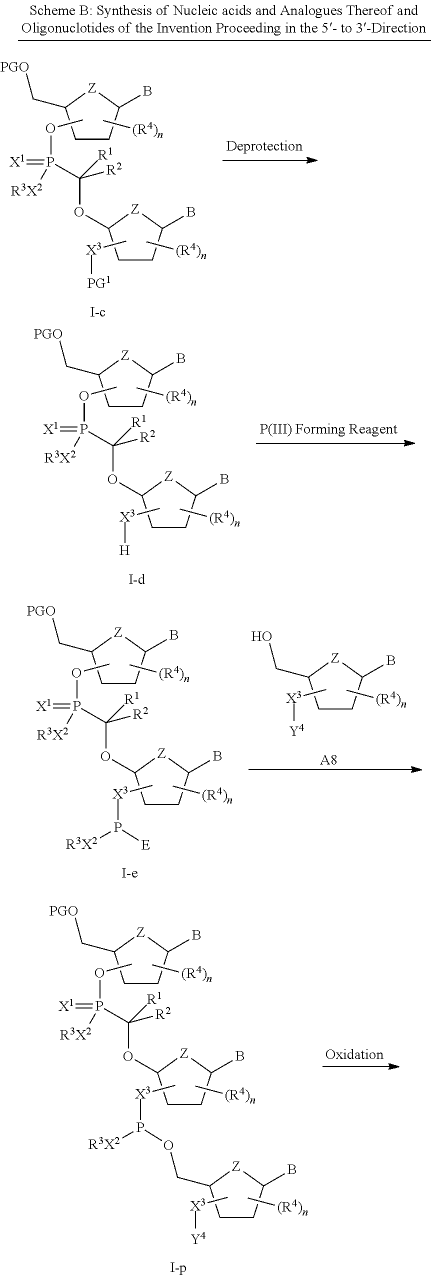

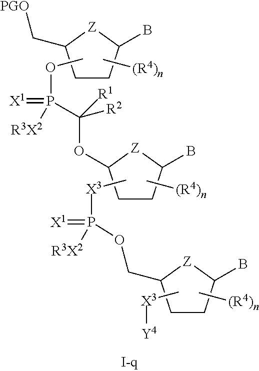

As depicted in Scheme B above, a nucleic acid or analogue thereof of formula I-c comprising, but not limited to, a 4′-O-methylene phosphonate internucleotide linkage of the invention, is first selectively deprotected to form nucleic acid or analogue thereof of formula I-d of the invention and then reacted with a P(III) forming reagent to form a nucleic acid or analogue thereof of formula I-e of the invention. Guidance to the choice of PG¹ and PG in a nucleic acid or analogue thereof of formula I-c to allow selective removal of PG¹ is provided within the current disclosure and is described in detail in Protecting Groups in Organic Synthesis, T. W. Greene and P. G. M. Wuts, 3^{rd} edition, John Wiley & Sons, 1999, the entirety of each of which is herein incorporated by reference. Nucleic acid or analogue thereof of formula I-e comprising, but not limited to, a 4′-O-methylene phosphonate internucleotide linkage of the invention can be then condensed with a nucleotide or analogue thereof of formula A8 to form nucleic acid or analogue thereof of formula I-p comprising, but not limited to, a 4′-O-methylene phosphonate internucleotide linkage of the invention. Oxidation of nucleic acid or analogue thereof of formula I-p then affords an oligonucleotide compound of formula I-q comprising, but not limited to, a 4′-O-methylene phosphonate internucleotide linkage of the invention. Each of B, E, PG, PG¹, R¹, R², R³, R⁴, X¹, X², X³, Y⁴, Z, and n is as defined above and described herein.

One of skill in the art will appreciate that various functional groups present in the nucleic acid or analogues thereof of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See for example, “March's Advanced Organic Chemistry”, 5^{th} Ed., Ed.: Smith, M. B. and March, J., John Wiley & Sons, New York: 2001, the entirety of each of which is herein incorporated by reference. Such interconversions may require one or more of the aforementioned techniques, and certain methods for synthesizing the provided nucleic acids of the invention are described below in the Exemplification.

According to one aspect, the present invention provides a method for preparing an oligonucleotide compound comprising a 4′-O-methylene phosphonate internucleotide linkage, wherein the 4′-O-methylene phosphonate internucleotide linkage is represented by formula I-i:

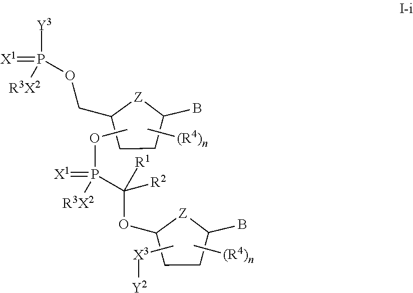

or a pharmaceutically acceptable salt thereof, comprising the steps:
(a) providing a nucleic acid or analogue thereof comprising a 4′-O-methylene phosphonate internucleotide linkage, wherein the 4′-O-methylene phosphonate internucleotide linkage is represented by formula I-h:

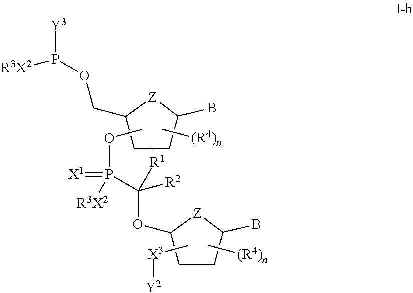

or a pharmaceutically acceptable salt thereof, and
(b) oxidizing the nucleic acid or analogue thereof comprising formula I-h to form the oligonucleotide compound comprising formula I-i, wherein:
each B is a nucleobase or hydrogen;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each X¹ is independently O, S, or NR;
each X² is independently —O—, —S—, —B(H)₂—, or a covalent bond;
X³ is —O—, —S—, —Se—, or —N(R)—;
Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
Y³ is a linking group attaching to the 2′- or 3′-terminal of a nucleotide, a nucleoside, or an oligonucleotide;
each Z is independently —O—, —S—, —N(R)—, or —C(R)₂—; and
each n is independently 0, 1, 2, 3, 4, or 5.

According to one aspect, the present invention provides a method for preparing an oligonucleotide compound comprising a 4′-O-methylene phosphonate internucleotide linkage, wherein the 4′-O-methylene phosphonate internucleotide linkage is represented by formula I-q:

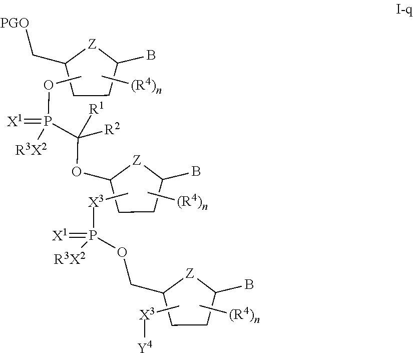

or a pharmaceutically acceptable salt thereof, comprising the steps:
(a) providing a nucleic acid or analogue thereof comprising a 4′-O-methylene phosphonate internucleotide linkage, wherein the 4′-O-methylene phosphonate internucleotide linkage is represented by formula I-p:

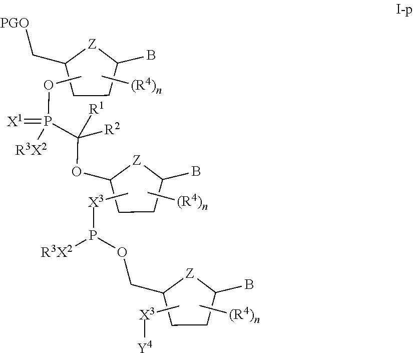

or a pharmaceutically acceptable salt thereof, and
(b) oxidizing the nucleic acid or analogue thereof comprising formula I-p to form the oligonucleotide compound comprising formula I-q, wherein:
each B is a nucleobase or hydrogen;
PG is a suitable hydroxyl protecting group;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each X¹ is independently O, S, or NR;
each X² is independently —O—, —S—, —B(H)₂—, or a covalent bond;
each X³ is independently —O—, —S—, —Se—, or —N(R)—;
Y⁴ is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
each Z is independently —O—, —S—, —N(R)—, or —C(R)₂—; and
each n is independently 0, 1, 2, 3, 4, or 5.

The oxidation of nucleic acid or analogue thereof comprising formula I-h to form oligonucleotide compound comprising formula I-i or nucleic acid or analogue thereof comprising formula I-p to form oligonucleotide compound comprising formula I-q can be performed using known oxidation conditions. The person skilled in the art will recognize that oxidation of P(III) to P(V) can be carried out by a variety of reagents, such as hydrogen peroxide, hydroperoxides, peroxides, peracids, iodine, and mixtures thereof. Hydrogen peroxide may be used in the presence of a solvent such as acetonitrile. Hydroperoxides (i.e., ROOH), include peroxides where R is alkyl or aryl and its salts, including but not limited to t-butyl peroxide (tBuOOH). Peroxides include alkyl, aryl, or mixed alkyl/aryl peroxides, and salts thereof. Peracids include, but are not limited to, alkyl and aryl peracids, including chloroperoxybenzoic acid (mCPBA). The use of basic halogens such as bromine (Br₂), chlorine (Cl₂) or iodine (I₂) can be performed in the presence of water and other components such as pyridine, tetrahydrofuran and water. Alternatively, aqueous Cl₂ solutions in the presence of TEMPO are also contemplated. Thus, the term “oxidizing agent” includes “sulfurizing agent,” which is also considered to have the same meaning as “thiation reagent.” Examples of sulfurization reagents which have been used to synthesize oligonucleotides containing phosphorothioate (PS) bonds include elemental sulfur, dibenzoyltetrasulfide, 3-H-1,2-benzidithiol-3-one 1,1-dioxide (Beaucage reagent), tetraethylthiuram disulfide (TETD), and bis(O,O-diisopropoxy phosphinothioyl) disulfide (Stec reagent). Oxidizing reagents for making phosphorothioate diester linkages include phenylacetyldisulfide (PADS), as described by Cole et al. in U.S. Pat. No. 6,242,591. In certain embodiments, the oxidation is performed using iodine in aqueous pyridine.

In certain aspects, the present invention provides a method for preparing an oligonucleotide compound comprising a 4′-O-methylene phosphonate internucleotide linkage, wherein the 4′-O-methylene phosphonate internucleotide linkage is represented by formula I-i-1:

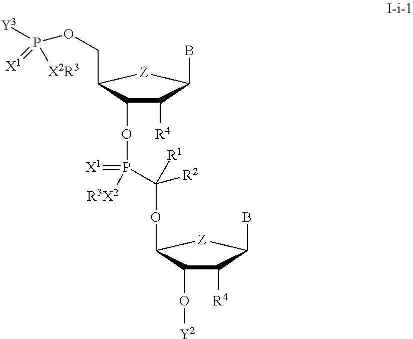

or a pharmaceutically acceptable salt thereof, comprising the steps:
(a) providing a nucleic acid or analogue thereof comprising a 4′-O-methylene phosphonate internucleotide linkage, wherein the 4′-O-methylene phosphonate internucleotide linkage is represented by formula I-h-1:

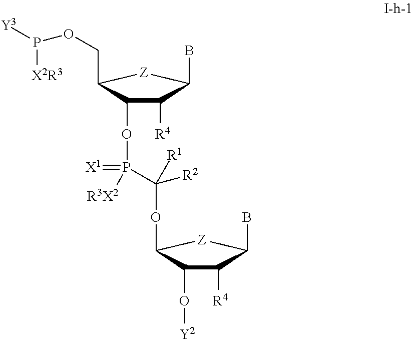

or pharmaceutically acceptable salt thereof, and
(b) oxidizing the nucleic acid or analogue thereof comprising formula I-h-1 to form the oligonucleotide compound comprising I-i-1, wherein:
each of B, R¹, R², R³, R⁴, X¹, X², Y², Y³, and Z is as described herein and defined above.

In certain aspects, the present invention provides a method for preparing an oligonucleotide compound comprising a 4′-O-methylene phosphonate internucleotide linkage, wherein the 4′-O-methylene phosphonate internucleotide linkage is represented by formula I-q-1:

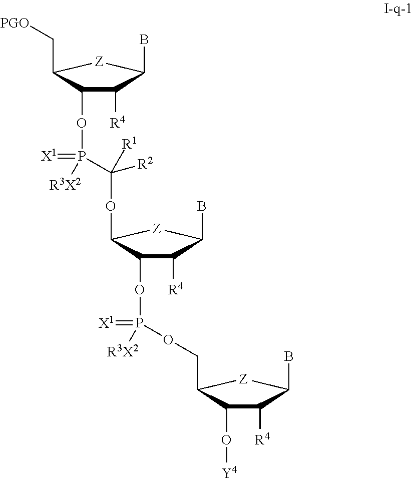

or a pharmaceutically acceptable salt thereof, comprising the steps:
(a) providing a nucleic acid or analogue thereof comprising a 4′-O-methylene phosphonate internucleotide linkage, wherein the 4′-O-methylene phosphonate internucleotide linkage is represented by formula I-p-1:

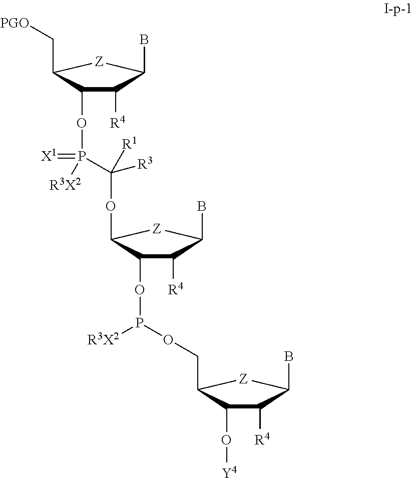

or pharmaceutically acceptable salt thereof, and
(b) oxidizing the nucleic acid or analogue thereof comprising formula I-p-1 to form the oligonucleotide compound comprising formula I-q-1, wherein:
each of B, PG, R¹, R², R³, R⁴, X¹, X², Y⁴, and Z is as described herein and defined above.

According to one aspect, the present invention provides a method for preparing a nucleic acid or analogue thereof comprising a 4′-O-methylene phosphonate internucleotide linkage, wherein the 4′-O-methylene phosphonate internucleotide linkage is represented by formula I-h:

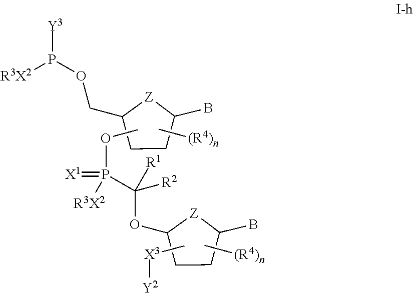

or a pharmaceutical acceptable salt thereof, comprising the steps:
(a) providing a nucleic acid or analogue thereof comprising a 4′-O-methylene phosphonate internucleotide linkage, wherein the 4′-O-methylene phosphonate internucleotide linkage is represented by formula I-g:

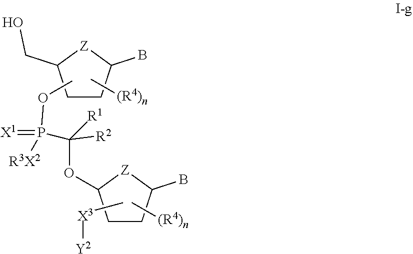

(b) reacting the nucleic acid or analogue thereof comprising formula I-g with a phosphoramidite analogue of formula A6:

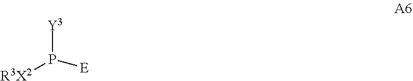

to form the nucleic acid or analogue thereof comprising formula I-h, wherein:
each B is a nucleobase or hydrogen;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
E is a halogen or —NR₂;
X¹ is O, S, or NR;
each X² is independently —O—, —S—, —B(H)₂—, or a covalent bond;
X³ is —O—, —S—, —Se—, or —N(R)—;
Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
Y³ is a linking group attaching to the 2′- or 3′-terminal of a nucleoside, a nucleotide, or an oligonucleotide;
each Z is independently —O—, —S—, —N(R)—, or —C(R)₂—; and
each n is independently 0, 1, 2, 3, 4, or 5.

According to one aspect, the present invention provides a method for preparing a nucleic acid or analogue thereof of formula I-e:

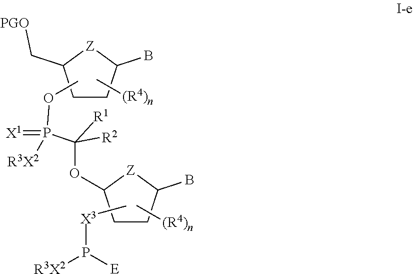

or a pharmaceutical acceptable salt thereof, comprising the steps:
(a) providing a nucleic acid or analogue thereof of formula I-d:

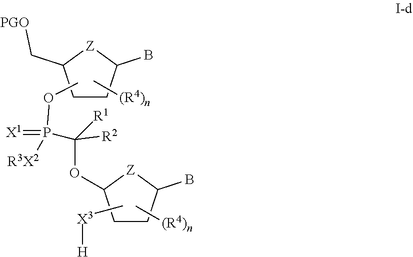

(b) reacting the nucleic acid or analogue thereof of formula I-d with a P(III) forming reagent to form the nucleic acid or analogue thereof of formula I-e, wherein:
each B is a nucleobase or hydrogen;
PG is a suitable hydroxyl protecting group;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
E is a halogen or —NR₂;
X¹ is O, S, or NR;
each X² is independently —O—, —S—, —B(H)₂—, or a covalent bond;
X³ is —O—, —S—, —Se—, or —N(R)—;
each Z is independently —O—, —S—, —N(R)—, or —C(R)₂—; and
each n is independently 0, 1, 2, 3, 4, or 5.

According to one embodiment, the phosphoramidite analogue of formula A6 in step (b) above is a nucleoside, a nucleotide, or an oligonucleotide comprising a phosphoramidite moiety commonly used in phosphoramidite oligonucleotide syntheses. In some embodiments, phosphoramidites or analogues thereof are prepared using a P(III) forming reagent. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) analogue in step (b) by the hydroxyl or X³ moiety of a nucleic acid or analogue thereof comprising formula I-d or formula I-g, respectively, is achieved either with or without the presence of a suitable base. Such suitable bases are well known in the art and include organic and inorganic bases. In some embodiments, the base is a tertiary amine such as triethylamine or diisopropylethylamine. In certain embodiments, the base is 4,5-dicyanoimidazole.

In certain aspects, the present invention provides a method for preparing a nucleic acid or analogue thereof comprising a 4′-O-methylene phosphonate internucleotide linkage, wherein the 4′-O-methylene phosphonate internucleotide linkage is represented by formula I-h-1:

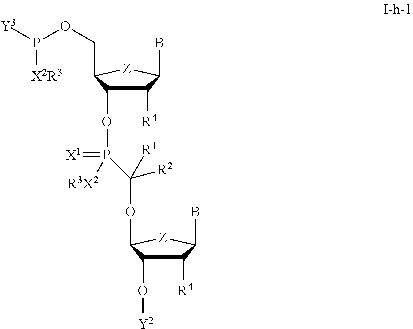

or a pharmaceutically acceptable salt thereof, comprising the steps:
(a) providing a nucleic acid or analogue thereof comprising a 4′-O-methylene phosphonate internucleotide linkage, wherein the 4′-O-methylene phosphonate internucleotide linkage is represented by formula I-g-1:

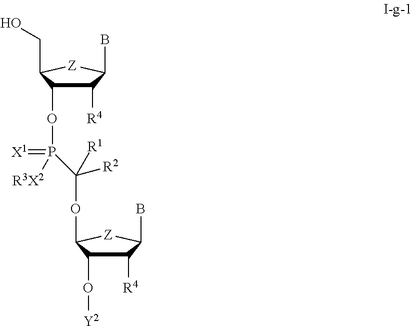

or pharmaceutically acceptable salt thereof, and
(b) reacting the nucleic acid or analogue thereof comprising formula I-g-1 with a phosphoramidite analogue of formula A6:

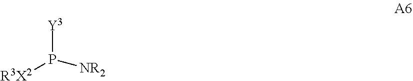

to form the nucleic acid or analogue thereof comprising formula I-h-1, wherein:
each of B, E, R¹, R², R³, R⁴, X¹, X², Y², Y³, and Z is as described herein and defined above.

In certain aspects, the present invention provides a method for preparing a nucleic acid or analogue thereof of formula I-e-1:

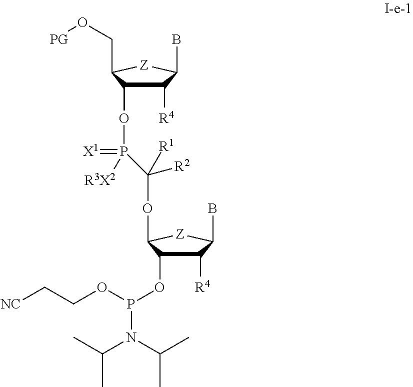

or a pharmaceutically acceptable salt thereof, comprising the steps:
(a) providing a nucleic acid or analogue thereof of formula I-d-1:

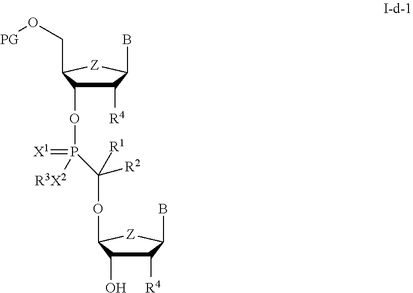

or pharmaceutically acceptable salt thereof, and
(b) reacting the nucleic acid or analogue thereof of formula I-d-1 with a P(III) forming reagent to form the nucleic acid or analogue thereof of formula I-e-1, wherein:
each of B, PG, R¹, R², R³, R⁴, X¹, X², and Z is as described herein and defined above.

According to one aspect, the present invention provides a method for preparing a nucleic acid or analogue thereof comprising a 4′-O-methylene phosphonate internucleotide linkage, wherein the 4′-O-methylene phosphonate internucleotide linkage is represented by formula I-g:

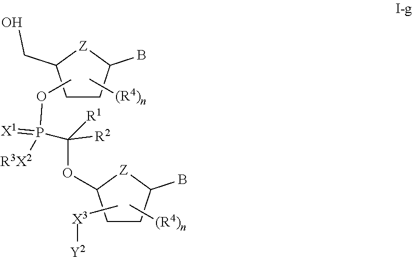

or a pharmaceutically acceptable salt thereof, comprising the steps:
(a) providing a nucleic acid or analogue thereof comprising a 4′-O-methylene phosphonate internucleotide linkage, wherein the 4′-O-methylene phosphonate internucleotide linkage is represented by formula I-b:

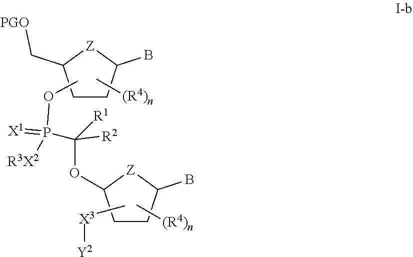

or pharmaceutically acceptable salt thereof, and
(b) deprotecting the nucleic acid or analogue thereof comprising formula I-b to form the nucleic acid or analogue thereof comprising formula I-g, wherein:
each B is a nucleobase or hydrogen;
PG is a suitable hydroxyl protecting group;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X¹ is O, S, or NR;
each X² is independently —O—, —S—, —B(H)₂—, or a covalent bond;
X³ is —O—, —S—, —Se—, or —N(R)—;
Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
each Z is independently —O—, —S—, —N(R)—, or —C(R)₂—; and
each n is independently 0, 1, 2, 3, 4, or 5.

According to one aspect, the present invention provides a method for preparing a nucleic acid of formula I-d:

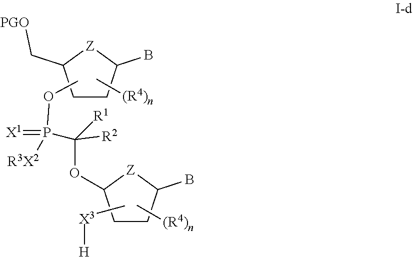

or a pharmaceutically acceptable salt thereof, comprising the steps:
(a) providing a nucleic acid or analogue thereof comprising of formula I-c:

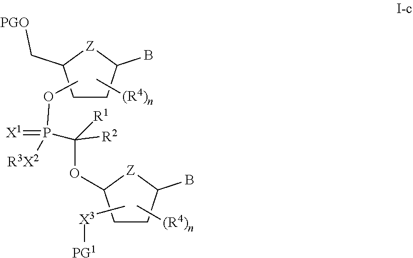

or pharmaceutically acceptable salt thereof, and
(b) deprotecting the nucleic acid or analogue thereof comprising formula I-d to form the nucleic acid or analogue thereof comprising formula I-c, wherein:
each B is a nucleobase or hydrogen;
PG is a suitable hydroxyl protecting group;
PG¹ is a protecting group;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X¹ is O, S, or NR;
each X² is independently —O—, —S—, —B(H)₂—, or a covalent bond;
X³ is —O—, —S—, —Se—, or —N(R)—;
each Z is independently —O—, —S—, —N(R)—, or —C(R)₂—; and
each n is independently 0, 1, 2, 3, 4, or 5.

According to embodiments described herein, the deprotection of a protecting group (e.g., PG or PG¹) in steps (b) above includes those protecting groups described in detail in Protecting Groups in Organic Synthesis, T. W. Greene and P. G. M. Wuts, 3^{rd} edition, John Wiley & Sons, 1999, the entirety of each of which is herein incorporated by reference. In some embodiments, the protecting group is a suitable hydroxyl protecting group, a suitable amino protection group, or a suitable thiol protecting group.

As used herein, the phrase “suitable hydroxyl protecting group” are well known in the art and when taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl, 3,4-dimethoxybenzyl, O-nitrobenzyl, p-nitrobenzyl, p-halobenzyl, 2,6-dichlorobenzyl, p-cyanobenzyl, and 2- and 4-picolyl. In some embodiments, the suitable hydroxyl protecting group is an acid labile group such as trityl, 4-methyoxytrityl, 4,4′-dimethyoxytrityl (DMTr), 4,4′,4″-trimethyoxytrityl, 9-phenyl-xanthen-9-yl, 9-(p-tolyl)-xanthen-9-yl, pixyl, 2,7-dimethylpixyl, and the like, suitable for deprotection during both solution-phase and solid-phase synthesis of acid-sensitive oligonucleotides using for example, dichloroacetic acid, trichloroacetic acid, trifluoroacetic acid, or acetic acid. The t-butyldimethylsilyl group is stable under the acidic conditions used to remove the DMTr group during synthesis but can be removed after cleavage and deprotection of the RNA oligomer with a fluoride source, e.g., tetrabutylammonium fluoride or pyridine hydrofluoride.

As used herein, the phrase “suitable amino protecting group” are well known in the art and when taken with the nitrogen to which it is attached, include, but are not limited to, aralkylamines, carbamates, allyl amines, amides, and the like. Examples of mono-protection groups for amines include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like. Examples of di-protection groups for amines include amines that are substituted with two substituents independently selected from those described above as mono-protection groups, and further include cyclic imides, such as phthalimide, maleimide, succinimide, 2,2,5,5-tetramethyl-1,2,5-azadisilolidine, azide, and the like. It will be appreciated that upon acid hydrolysis of an amino protecting groups, a salt compound thereof is formed. For example, when an amino protecting group is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms are contemplated.

As used herein, the phrase “suitable thiol protecting group” further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

In certain aspects, the present invention provides a method for preparing a nucleic acid or analogue thereof comprising a 4′-O-methylene phosphonate internucleotide linkage, wherein the 4′-O-methylene phosphonate internucleotide linkage is represented by formula I-g-1:

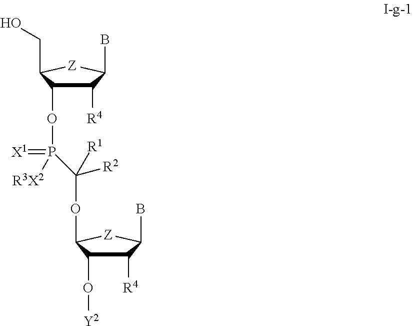

or a pharmaceutically acceptable salt thereof, comprising the steps:
(a) providing a nucleic acid or analogue thereof comprising a 4′-O-methylene phosphonate internucleotide linkage, wherein the 4′-O-methylene phosphonate internucleotide linkage is represented by formula I-b-1:

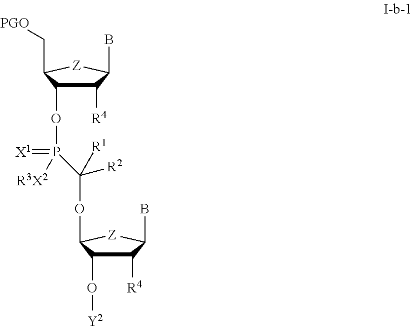

or pharmaceutically acceptable salt thereof, and
(b) deprotecting the nucleic acid or analogue thereof comprising formula I-b-1 to form the nucleic acid or analogue thereof comprising formula I-g-1, wherein:
each of B, PG, R¹, R², R³, R⁴, X¹, X², Y², and Z is as described herein and defined above.

In certain aspects, the present invention provides a method for preparing a nucleic acid or analogue thereof of formula I-d-1:

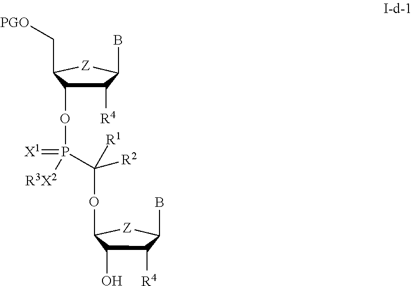

or a pharmaceutically acceptable salt thereof, comprising the steps:
(a) providing a nucleic acid or analogue thereof of formula I-c-1:

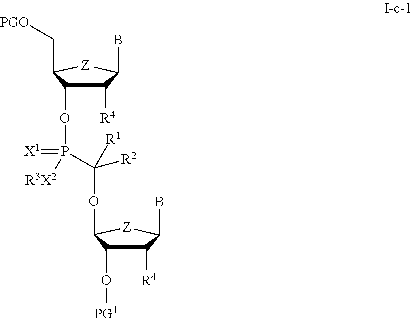

or pharmaceutically acceptable salt thereof, and
(b) deprotecting the nucleic acid or analogue thereof of formula I-d-1 to form the nucleic acid or analogue thereof of formula I-c-1, wherein:
each of B, PG, PG¹, R¹, R², R³, R⁴, X¹, X², and Z is as described herein and defined above.

According to one aspect, the present invention provides a method for preparing a nucleic acid or analogue thereof comprising a 4′-O-methylene phosphonate internucleotide linkage, wherein the 4′-O-methylene phosphonate internucleotide linkage is represented by formula I-b:

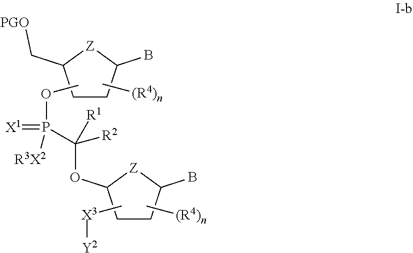

or a pharmaceutically acceptable salt thereof, comprising the steps:
(a) providing a nucleic acid or analogue thereof of formula A4:

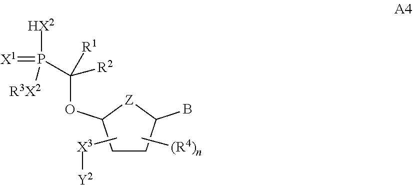

or pharmaceutically acceptable salt thereof, and
(b) condensing the nucleic acid or analogue thereof of formula A4 with a nucleoside or analogue thereof of formula A5:

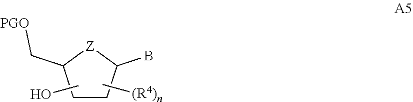

to form the nucleic acid or analogue thereof comprising formula I-b, wherein:
each B is a nucleobase or hydrogen;
PG is a suitable hydroxyl protecting group;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X¹ is O, S, or NR;
each X² is independently —O—, —S—, —B(H)₂—, or a covalent bond;
X³ is —O—, —S—, —Se—, or —N(R)—;
Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
each Z is independently —O—, —S—, —N(R)—, or —C(R)₂—; and
each n is independently 0, 1, 2, 3, 4, or 5.

According to one aspect, the present invention provides a method for preparing a nucleic acid or analogue thereof comprising a 4′-O-methylene phosphonate internucleotide linkage, wherein the 4′-O-methylene phosphonate internucleotide linkage is represented by formula I-p:

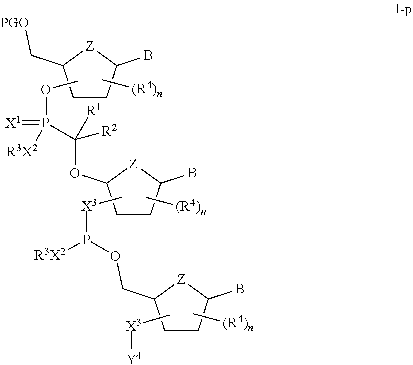

or a pharmaceutically acceptable salt thereof, comprising the steps:
(a) providing a nucleic acid or analogue thereof of formula I-e:

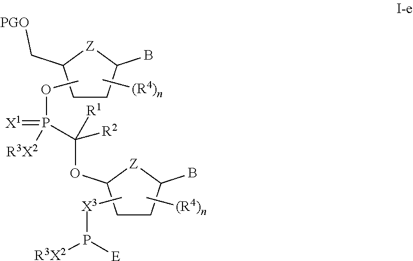

or pharmaceutically acceptable salt thereof, and
(b) condensing the nucleic acid or analogue thereof of formula I-e with a nucleoside or analogue thereof of formula A8:

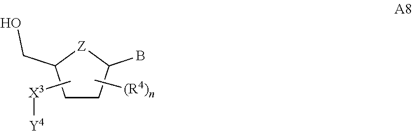

to form the nucleic acid or analogue thereof comprising formula I-p, wherein:
each B is a nucleobase or hydrogen;
PG is a suitable hydroxyl protecting group;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
E is a halogen or —NR₂;
X¹ is O, S, or NR;
each X² is independently —O—, —S—, —B(H)₂—, or a covalent bond;
X³ is —O—, —S—, —Se—, or —N(R)—;
Y⁴ is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
each Z is independently —O—, —S—, —N(R)—, or —C(R)₂—; and
each n is independently 0, 1, 2, 3, 4, or 5.

According to some embodiments, the condensation in steps (b) above include the use of a condensing agent. The condensing agent used for the condensation of the nucleic acid or analogue thereof of formula A4 with a nucleoside or analogue thereof of formula A5 or nucleic acid or analogue thereof comprising formula I-e with a nucleoside or analogue thereof of formula A8, may include sulfonyl chlorides such as methanesulfonyl chloride, toluenesulfonyl chloride, 2,4,6-triisopropylbenzenesulfonyl chloride, or mesitylene-2-sulfonyl chloride; sulfonyltetrazoles such as 1-toluenesulfonyltetrazole, 1-(mesitylene-2-sulfonyl)tetrazole, or 1-(2,4,6-triisopropylbenzenesulfonyl)tetrazole; sulfonyltriazoles such as 3-nitro-1-toluenesulfonyl-1,2,4-triazole, 3-nitro-1-(mesitylene-2-sulfonyl)-1,2,4-triazole, or 3-nitro-1-(2,4,6-triisopropylbenezenesulfonyl)-1,2,4-triazole; or the like. In certain embodiments, the condensing agent is triisopropylbenzenesulfonyl chloride. During the condensation, a base may be co-present. Examples of the base used therefor include triethylamine, ethyldiisopropylamine, pyridine, lutidine, imidazole, N-methylimidazole, N-methylbenzimidazole, or the like. In certain embodiments, the base is N-methylimidazole.

In certain aspects, the present invention provides a method for preparing a nucleic acid or analogue thereof comprising a 4′-O-methylene phosphonate internucleotide linkage, wherein the 4′-O-methylene phosphonate internucleotide linkage is represented by formula I-b-1:

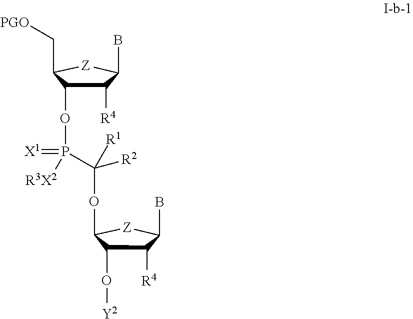

or a pharmaceutically acceptable salt thereof, comprising the steps:
(a) providing a nucleic acid or analogue thereof of formula A4-1:

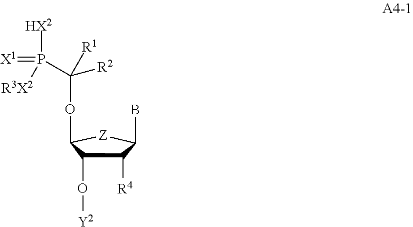

or pharmaceutically acceptable salt thereof, and
(b) condensing the nucleic acid or analogue thereof comprising formula A4-1 with a nucleoside or analogue thereof of formula A5-1:

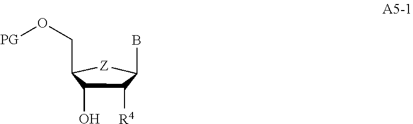

to form the nucleic acid or analogue thereof comprising formula I-b-1, wherein:
each of B, PG, R¹, R², R³, R⁴, X¹, X², Y², and Z is as described herein and defined above.

In certain aspects, the present invention provides a method for preparing a nucleic acid or analogue thereof comprising a 4′-O-methylene phosphonate internucleotide linkage, wherein the 4′-O-methylene phosphonate internucleotide linkage is represented by formula I-p-1:

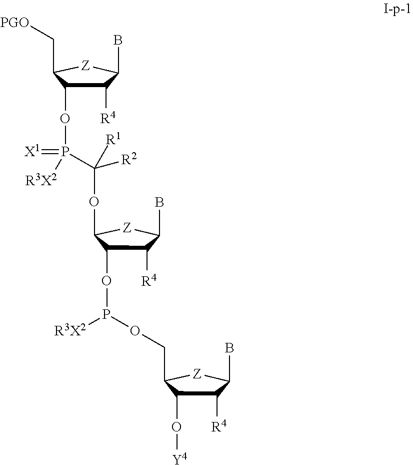

or a pharmaceutically acceptable salt thereof, comprising the steps:
(a) providing a nucleic acid or analogue thereof of formula I-e-1:

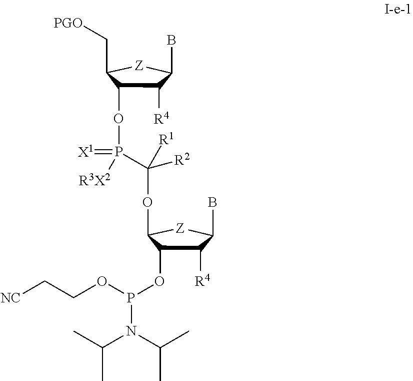

or pharmaceutically acceptable salt thereof, and
(b) condensing the nucleic acid or analogue thereof of formula I-e-1 with a nucleoside or analogue thereof of formula A8-1:

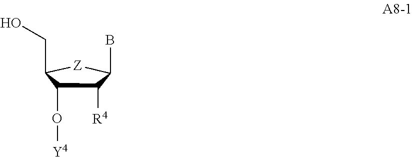

to form the nucleic acid or analogue thereof comprising formula I-p-1, wherein:
each of B, PG, R¹, R², R³, R⁴, X¹, X², Y⁴, and Z is as described herein and defined above.

According to one aspect, the present invention provides a method for preparing an oligonucleotide compound comprising a 4′-O-methylene phosphonate internucleotide linkage, wherein the 4′-O-methylene phosphonate internucleotide linkage is represented by formula A4:

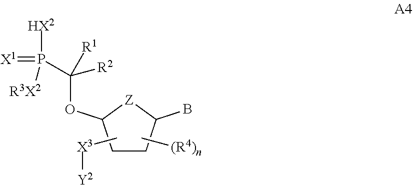

or a pharmaceutically acceptable salt thereof, comprising the steps:
(a) providing a nucleic acid or analogue thereof of formula A3:

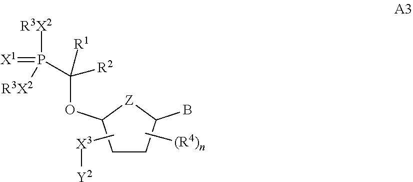

or pharmaceutically acceptable salt thereof, and
(b) deprotecting the nucleic acid or analogue thereof of formula A3 to form the nucleic acid or analogue thereof of formula A4, wherein:
B is a nucleobase or hydrogen;
R¹ and R² are independently hydrogen, halogen, R⁵, —CN, —S(O)R, —S(O)₂R, —Si(OR)₂R, —Si(OR)R₂, or —SiR₃, or:
   R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X¹ is O, S, or NR;
each X² is independently —O—, —S—, —B(H)₂—, or a covalent bond;
X³ is —O—, —S—, —Se—, or —N(R)—;
Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
Z is —O—, —S—, —N(R)—, or —C(R)₂—; and
n is 0, 1, 2, 3, 4, or 5.

In certain embodiments, Y² is a protecting group.

According to embodiments described herein, the deprotection of formula A3 in step (b) above can include the deprotection of any suitable protection group disclosed above or defined herein. In certain embodiments, the nucleic acid or analogue of formula A3 comprises a 4′-O-methylene phosphonate ester and mono-deprotection is performed under basic aqueous conditions. Suitable bases metal hydroxides (e.g., sodium hydroxide, potassium hydroxide, lithium hydroxide and barium hydroxide), metal carbonates (e.g., lithium carbonate, sodium carbonate, potassium carbonate, calcium carbonate, cesium carbonate), sodium hydrogen carbonate, organic amines (e.g., triethylamine, N,N-diisopropylethylamine (DIEA), N-methylmorpholine, N-ethylmorpholine, tributylamine, 1,4-diazabicyclo[2.2.2]octane (DABCO), N-methylimidazole (NMI), pyridine, 2,6-lutidine, 2,4,6-collidine, 4-dimethylaminopyridine (DMAP), 1,8-bis(dimethylamino)naphthalene (“proton sponge”), 1,8-diazabicyclo[5.4.0]undec-7-ene (DBU), 1,5-diazabicyclo[4.3.0]non-5-ene (DBN), 7-methyl-1,5,7-triazabicyclo[4.4.0]dec-5-ene (MTBD), 2-tert-butyl-1,1,3,3-tetramethylguanidine, 2,8,9-trimethyl-2,5,8,9-tetraaza-1-phosphabicyclo[3.3.3]undecane or phosphazene base).

In certain aspects, the present invention provides a method for preparing a nucleic acid or analogue thereof of formula A4-1:

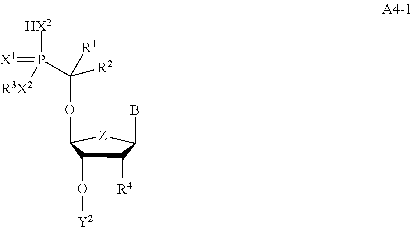

or a pharmaceutically acceptable salt thereof, comprising the steps:
(a) providing a nucleic acid or analogue thereof of formula A3-1:

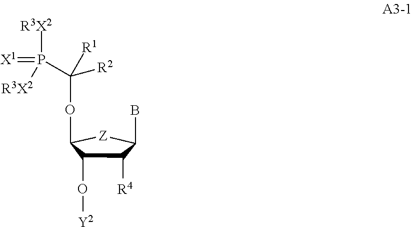

or pharmaceutically acceptable salt thereof, and
(b) deprotecting the nucleic acid or analogue thereof of formula A4-1 to form the nucleic acid or analogue thereof of formula A3-1, wherein:
each of B, PG, R¹, R², R³, R⁴, X¹, X², Y², and Z is as described herein and defined above.

In certain embodiments, Y² is a protecting group.

### 5. Uses, Formulation and Administration

Pharmaceutically Acceptable Compositions

According to another embodiment, the invention provides a composition comprising a nucleic acid or analogue thereof comprising a 4′-O-methylene phosphonate internucleotide linkage of this invention and a pharmaceutically acceptable carrier, adjuvant, or vehicle. The amount of a provided nucleic acid in the compositions of this invention is effective to measurably modulate the expression of a target gene in a biological sample or in a patient. In certain embodiments, a composition of this invention is formulated for administration to a patient in need of such composition. In some embodiments, a composition of this invention is formulated for parenteral or oral administration to a patient. In some embodiments, the composition comprises a pharmaceutically acceptable carrier, adjuvant, or vehicle, and a nucleic acid inhibitor molecule, wherein the nucleic acid inhibitor molecule comprises at least one nucleotide comprising a 4′-O-methylene phosphonate internucleotide linkage or analogue thereof, as described herein.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of a provided nucleic acid with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a provided nucleic acid of this invention that, upon administration to a recipient, is capable of providing, either directly or indirectly, a provided nucleic acid of this invention or an inhibitory active metabolite or residue thereof.

As used herein, the term “inhibitory active metabolite or residue thereof” means that a metabolite or residue thereof is also useful to modulate the expression of a target gene in a biological sample or in a patient.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are formulated in liquid form for parenteral administration, for example, by subcutaneous, intramuscular, intravenous or epidural injection. Dosage forms suitable for parenteral administration typically comprise one or more suitable vehicles for parenteral administration including, by way of example, sterile aqueous solutions, saline, low molecular weight alcohols such as propylene glycol, polyethylene glycol, vegetable oils, gelatin, fatty acid esters such as ethyl oleate, and the like. The parenteral formulations may contain sugars, alcohols, antioxidants, buffers, bacteriostats, solutes which render the formulation isotonic with the blood of the intended recipient or suspending or thickening agents. Proper fluidity can be maintained, for example, by the use of surfactants. Liquid formulations can be lyophilized and stored for later use upon reconstitution with a sterile injectable solution.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. Compositions of this invention formulated for oral administration may be administered with or without food. In some embodiments, pharmaceutically acceptable compositions of this invention are administered without food. In other embodiments, pharmaceutically acceptable compositions of this invention are administered with food.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be affected in a rectal suppository formulation (see above) or in a suitable enema formulation. Topically transdermal patches may also be used.

For topical applications, provided pharmaceutically acceptable compositions may be formulated in a suitable ointment containing the active component suspended or dissolved in one or more carriers. Carriers for topical administration of nucleic acid or analogues thereof of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, a provided nucleic acid (e.g., nucleic acid inhibitor molecule) may be admixed, encapsulated, conjugated or otherwise associated with other molecules, molecule structures or mixtures of compounds, including, for example, liposomes and lipids such as those disclosed in U.S. Pat. Nos. 6,815,432, 6,586,410, 6,858,225, 7,811,602, 7,244,448 and 8,158,601; polymeric materials such as those disclosed in U.S. Pat. Nos. 6,835,393, 7,374,778, 7,737,108, 7,718,193, 8,137,695 and U.S. Published Patent Application Nos. 2011/0143434, 2011/0129921, 2011/0123636, 2011/0143435, 2011/0142951, 2012/0021514, 2011/0281934, 2011/0286957 and 2008/0152661; capsids, capsoids, or receptor targeted molecules for assisting in uptake, distribution or absorption, the entirety of each of which is herein incorporated by reference.

In certain embodiments, a provided nucleic acid (e.g., nucleic acid inhibitor molecule) is formulated in a lipid nanoparticle (LNP). Lipid-nucleic acid nanoparticles typically form spontaneously upon mixing lipids with nucleic acid to form a complex. Depending on the desired particle size distribution, the resultant nanoparticle mixture can be optionally extruded through a polycarbonate membrane (e.g., 100 nm cut-off) using, for example, a thermobarrel extruder, such as LIPEX® Extruder (Northern Lipids, Inc). To prepare a lipid nanoparticle for therapeutic use, it may desirable to remove solvent (e.g., ethanol) used to form the nanoparticle and/or exchange buffer, which can be accomplished by, for example, dialysis or tangential flow filtration. Methods of making lipid nanoparticles containing nucleic acid inhibitor molecules are known in the art, as disclosed, for example in U.S. Published Patent Application Nos. 2015/0374842 and 2014/0107178, the entirety of each of which is herein incorporated by reference.

In certain embodiments, the LNP comprises a lipid core comprising a cationic liposome and a pegylated lipid. The LNP can further comprise one or more envelope lipids, such as a cationic lipid, a structural or neutral lipid, a sterol, a pegylated lipid, or mixtures thereof.

In certain embodiments, a provided nucleic acid is covalently conjugated to a ligand that directs delivery of the nucleic acid to a tissue of interest. Many such ligands have been explored. See, e.g., Winkler, THER. DELIV., 2013, 4(7): 791-809. For example, a provided nucleic acid can be conjugated to multiple sugar ligand moieties (e.g., N-acetylgalactosamine (GalNAc)) to direct uptake of the nucleic acid into the liver. See, e.g., WO 2016/100401. Other ligands that can be used include, but are not limited to, mannose-6-phosphate, cholesterol, folate, transferrin, and galactose (for other specific exemplary ligands see, e.g., WO 2012/089352). Typically, when a provided nucleic acid is conjugated to a ligand, the nucleic acid is administered as a naked nucleic acid, wherein the oligonucleotide is not also formulated in an LNP or other protective coating. In certain embodiments, each nucleotide within the naked nucleic acid is modified at the 2′-position of the sugar moiety, typically with 2′-F or 2′-OMe.

These pharmaceutical compositions may be sterilized by conventional sterilization techniques or may be sterile filtered. The resulting aqueous solutions may be packaged for use as is, or lyophilized, the lyophilized preparation being combined with a sterile aqueous excipient prior to administration. The pH of the preparations typically will be between 3 and 11, more preferably between 5 and 9 or between 6 and 8, and most preferably between 7 and 8, such as 7 to 7.5. The pharmaceutical compositions in solid form may be packaged in multiple single dose units, each containing a fixed amount of the above-mentioned agent or agents, such as in a sealed package of tablets or capsules. The pharmaceutical compositions in solid form can also be packaged in a container for a flexible quantity, such as in a squeezable tube designed for a topically applicable cream or ointment.

The amount of nucleic acid or analogue thereof of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the nucleic acid or analogue thereof can be administered to a patient receiving these compositions.

It should also be understood that a specific dosage and treatment regimen for any particular patient will depend upon a variety of factors, including the activity of the specific nucleic acid or analogue thereof employed, the age, body weight, general health, sex, diet, time of administration, rate of excretion, drug combination, and the judgment of the treating physician and the severity of the particular disease being treated. The amount of a nucleic acid or analogue thereof of the present invention in the composition will also depend upon the particular nucleic acid or analogue thereof in the composition.

Uses of Nucleic Acids and Analogues Thereof and Pharmaceutically Acceptable Compositions

Nucleic acids and analogues thereof and compositions described herein are generally useful for modulation of intracellular RNA levels. A provided nucleic acid comprising a 4′-O-methylene phosphonate internucleotide linkage or analogue thereof can be used in a method of modulating the expression of a target gene in a cell. Typically, such methods comprise introducing a provided nucleic acid inhibitor molecule into a cell in an amount sufficient to modulate the expression of a target gene. In certain embodiments, the method is carried out in vivo. The method can also be carried out in vitro or ex vivo. In certain embodiments, the cell is a mammalian cell, including, but not limited to, a human cell.

In certain embodiments, a provided nucleic acid comprising a 4′-O-methylene phosphonate internucleotide linkage or analogue thereof (e.g., nucleic acid inhibitor molecule) can be used in a method of treating a patient in need thereof. Typically, such methods comprise administering a therapeutically effective amount of a pharmaceutical composition comprising a provided nucleic acid inhibitor molecule, as described herein, to a patient in need thereof.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In certain embodiments, the pharmaceutical compositions disclosed herein may be useful for the treatment or prevention of symptoms related to a viral infection in a patient in need thereof. One embodiment is directed to a method of treating a viral infection, comprising administering to a subject a pharmaceutical composition comprising a therapeutically effective amount of a provided nucleic acid comprising a 4′-O-methylene phosphonate internucleotide linkage or analogue thereof (e.g., nucleic acid inhibitor molecule), as described herein. Non-limiting examples of such viral infections include HCV, HBV, HPV, HSV or HIV infection.

In certain embodiments, the pharmaceutical compositions disclosed herein may be useful for the treatment or prevention of symptoms related to cancer in a patient in need thereof. One embodiment is directed to a method of treating cancer, comprising administering to a subject a pharmaceutical composition comprising a therapeutically effective amount of a provided nucleic acid inhibitor molecule, as described herein. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, wilms tumor, leukemia, acute lymocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T-cell lymphomas, non-Hodgkin lymphoma, precursor T-lymphoblastic lymphoma/leukemia, peripheral T-cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Typically, the present disclosure features methods of treating liver cancer, liver carcinoma, hepatoma, hepatocellular carcinoma, cholangiocarcinoma and hepatoblastoma by administering a therapeutically effective amount of a pharmaceutical composition as described herein.

In certain embodiments the pharmaceutical compositions disclosed herein may be useful for treatment or prevention of symptoms related to proliferative, inflammatory, autoimmune, neurologic, ocular, respiratory, metabolic, dermatological, auditory, liver, kidney, or infectious diseases. One embodiment is directed to a method of treating a proliferative, inflammatory, autoimmune, neurologic, ocular, respiratory, metabolic, dermatological, auditory, liver, kidney, or infectious disease, comprising administering to a subject a pharmaceutical composition comprising a therapeutically effective amount of a provided nucleic acid inhibitor molecule, as described herein. Typically, the disease or condition is disease of the liver.

In some embodiments, the present disclosure provides a method for reducing expression of a target gene in a subject comprising administering a pharmaceutical composition to a subject in need thereof in an amount sufficient to reduce expression of the target gene, wherein the pharmaceutical composition comprises a provided nucleic acid inhibitor molecule comprising a 4′-O-methylene phosphonate internucleotide linkage or analogue thereof as described herein and a pharmaceutically acceptable excipient as also described herein.

In some embodiments, a provided nucleic acid inhibitor molecule is an RNAi inhibitor molecule as described herein, including a dsRNAi inhibitor molecule or an ssRNAi inhibitor molecule.

The target gene may be a target gene from any mammal, such as a human target gene. Any gene may be silenced according to the instant method. Exemplary target genes include, but are not limited to, Factor VII, Eg5, PCSK9, TPX2, apoB, SAA, TTR, HBV, HCV, RSV, PDGF beta gene, Erb-B gene, Src gene, CRK gene, GRB2 gene, RAS gene, MEKK gene, INK gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, p73 gene, p21(WAF1/CIP1) gene, p27(KIP1) gene, PPM1D gene, RAS gene, caveolin I gene, MIB I gene, MTAI gene, M68 gene, mutations in tumor suppressor genes, p53 tumor suppressor gene, LDHA, and combinations thereof.

In some embodiments, a provided nucleic acid inhibitor molecule comprising a 4′-O-methylene phosphonate internucleotide linkage or analogue thereof silences a target gene and thus can be used to treat a subject having or at risk for a disorder characterized by unwanted expression of the target gene. For example, in some embodiments, the provided nucleic acid inhibitor molecule silences the beta-catenin gene, and thus can be used to treat a subject having or at risk for a disorder characterized by unwanted beta-catenin expression, e.g., adenocarcinoma or hepatocellular carcinoma.

Typically, a provided nucleic acid (e.g., nucleic acid inhibitor molecule) of the invention are administered intravenously or subcutaneously. However, the pharmaceutical compositions disclosed herein may also be administered by any method known in the art, including, for example, oral, buccal, sublingual, rectal, vaginal, intraurethral, topical, intraocular, intranasal, and/or intra-auricular, which administration may include tablets, capsules, granules, aqueous suspensions, gels, sprays, suppositories, salves, ointments, or the like.

In certain embodiments, the pharmaceutical composition is delivered via systemic administration (such as via intravenous or subcutaneous administration) to relevant tissues or cells in a subject or organism, such as the liver. In other embodiments, the pharmaceutical composition is delivered via local administration or systemic administration. In certain embodiments, the pharmaceutical composition is delivered via local administration to relevant tissues or cells, such as lung cells and tissues, such as via pulmonary delivery.

The therapeutically effective amount of the nucleic acid or analogues thereof disclosed herein may depend on the route of administration and the physical characteristics of the patient, such as the size and weight of the subject, the extent of the disease progression or penetration, the age, health, and sex of the subject.

In certain embodiments, a provided nucleic acid, as described herein, is administered at a dosage of 20 micrograms to 10 milligrams per kilogram body weight of the recipient per day, 100 micrograms to 5 milligrams per kilogram body weight of the recipient per day, or 0.5 to 2.0 milligrams per kilogram body weight of the recipient per day.

A pharmaceutical composition of the instant disclosure may be administered every day or intermittently. For example, intermittent administration of a nucleic acid or analogues thereof of the instant disclosure may be administration one to six days per week, one to six days per month, once weekly, once every other week, once monthly, once every other month, or once or twice per year or divided into multiple yearly, monthly, weekly, or daily doses. In some embodiments, intermittent dosing may mean administration in cycles (e.g. daily administration for one day, one week or two to eight consecutive weeks, then a rest period with no administration for up to one week, up to one month, up to two months, up to three months or up to six months or more) or it may mean administration on alternate days, weeks, months or years.

In any of the methods of treatment of the invention, the nucleic acid or analogues thereof may be administered to the subject alone as a monotherapy or in combination with additional therapies known in the art.

### EXEMPLIFICATION

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis (pinacolato)diboron-4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DMTr: 4,4′-dimethyoxytrityl
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
L-DBTA: dibenzoyl-L-tartaric acid
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAF: tetrabutylammmonium fluoride
TBAI: tetrabutylammonium iodide
TEA: triethylamine
Tf: trifluoromethanesulfonate
TfAA, TFMSA or Tf₂O: trifluoromethanesulfonic anhydride
TFA: trifluoroacetic acid
TIBSCl: 2,4,6-triisopropylbenzenesulfonyl chloride
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
UPLC: Ultra Performance Liquid Chromatography
wt: weight
Xantphos: 4,5-bis(diphenylphosphino)-9,9-dimethylxanthene

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the nucleic acid or analogues thereof of the present invention are either commercially available or can be produced by organic synthesis methods known to one of ordinary skill in the art (Houben-Weyl 4th Ed. 1952, Methods of Organic Synthesis, Thieme, Volume 21). Further, the nucleic acid or analogues thereof of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain nucleic acid or analogues thereof disclosed herein, one or more ¹H shifts overlap with residual proteo solvent signals; these signals have not been reported in the experimental provided hereinafter.

As depicted in the Examples below, in certain exemplary embodiments, the nucleic acid or analogues thereof were prepared according to the following general procedures. It will be appreciated that, although the general methods depict the synthesis of certain nucleic acid or analogues thereof of the present invention, the following general methods, and other methods known to one of ordinary skill in the art, can be applied to all nucleic acid or analogues thereof and subclasses and species of each of these nucleic acid or analogues thereof, as described herein.

### Example 1. Synthesis of (2R,3S,4R,5R)-2-(((((2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl)oxy)(methoxy)phosphoryl)methoxy)-5-(2,4-dioxo-3,4-dihydropyrimidin-1(2H)-yl)-4-methoxytetrahydrofuran-3-yl (2-cyanoethyl) diisopropylphosphoramidite (I-3)

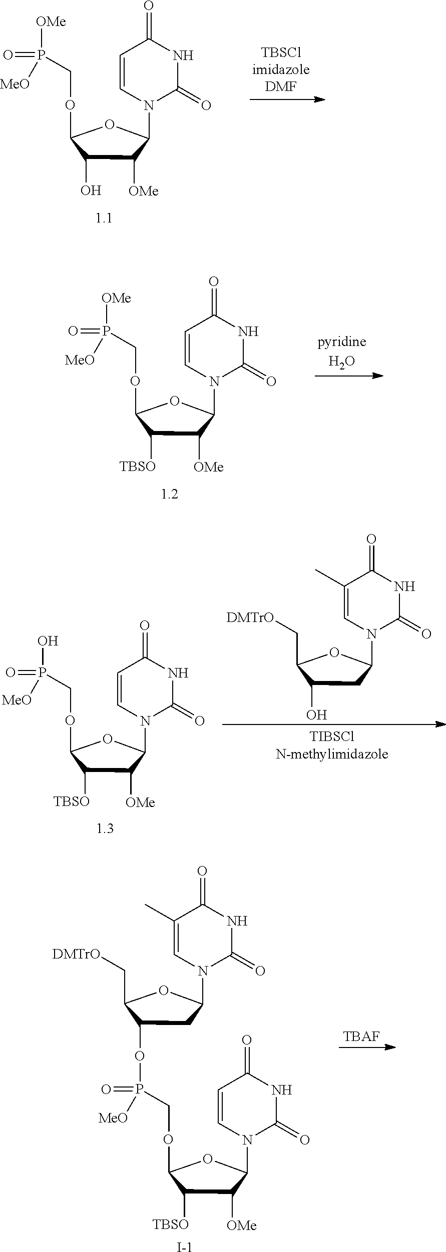

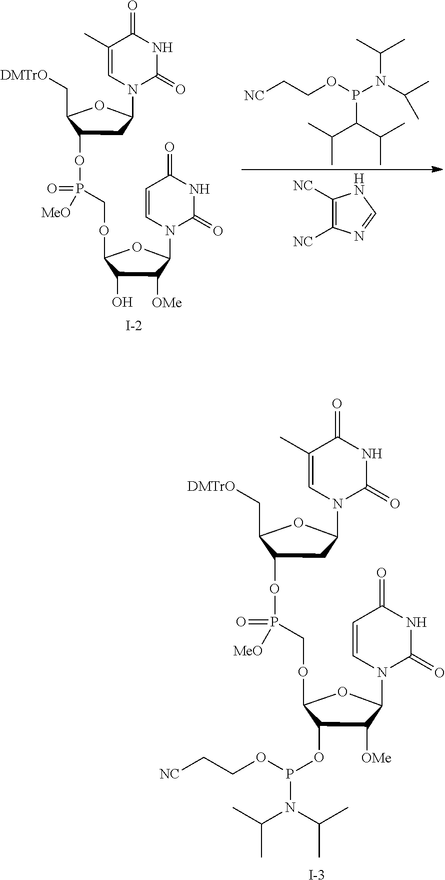

### Step 1: Dimethyl ((((2R,3S,4R,5R)-3-((tert-butyldimethylsilyl)oxy)-5-(2,4-dioxo-3,4-dihydropyrimidin-1(2H)-yl)-4-methoxytetrahydrofuran-2-yl)oxy)methyl)phosphonate. (1.2)

1.10 g of 1.1 was dissolved in 10 mL anhydrous DMF. 1.22 g of imidazole was then added to the solution. After adding 1.36 g of TBSCl, the reaction mixture was kept stirring at rt for 10 hours. After reaction completion as verified by UPLC, DMF was removed under reduced pressure. The yellowish residue was then re-dissolved in 200 mL EA and washed twice with 75 mL water and once with brine. The resulting solution was dried over anhydrous sodium sulfate and volatiles were removed by rotary evaporation. The crude product was then purified by flash column (0-10% MeOH in DCM) to afford 1.2 (1.21 g, 83% yield) as a white foam. ¹H-NMR (400 MHz, CDCl₃) δ (ppm): 8.42 (s, 1H), 7.59 (d, J=8 Hz, 1H), 7.58 (d, J=4 Hz, 1H), 6.33 (dd, J1=8 Hz, J2=1.6 Hz, 1H), 4.89 (s, 1H), 4.25 (d, J=4 Hz, 1H), 4.03 (m, 1H), 3.76-3.91 (m, 2H), 3.85 (d, J=4 Hz, 3H), 3.82 (d, J=4 Hz, 3H), 3.37 (s, 3H), 0.91 (s, 9H), 0.13 (s, 3H), 0.11 (s, 3H). MS (ESI) m/z calculated for C₁₈H₃₃N₂NaO₉PSi⁻ 503.5150, found: 503.55.

### Step 2: Methyl hydrogen ((((2R,3S,4R,5R)-3-((tert-butyldimethylsilyl)oxy)-5-(2,4-dioxo-3,4-dihydropyrimidin-1(2H)-yl)-4-methoxytetrahydrofuran-2-yl)oxy)methyl)phosphonate. (1.3)

3.30 g of 1.2 was dissolved in 120 mL aqueous pyridine (pyridine:water 3:2). The reaction mixture was heated to 50° C. and kept stirring for 16 hours. After reaction completion as verified by UPLC, the volatiles were removed under reduced pressure. The crude product 1.3 (3.74 g, quantitative) was used in the next step without further purification. ¹H-NMR (400 MHz, DMSO-d₆) δ (ppm): 11.16 (br, 1H), 7.75 (d, J=1 Hz, 1H), 6.09 (d, J=8 Hz, 1H), 5.67 (d, J1=8 Hz, 1H), 4.94 (s, 1H), 4.24 (d, J=4 Hz, 1H), 3.95 (m, 1H), 3.45-3.61 (m, 2H), 3.43 (d, J=4 Hz, 3H), 3.26 (s, 3H), 0.87 (s, 9H), 0.11 (s, 3H), 0.10 (s, 3H). ³¹P-NMR (200 MHz, DMSO-d₆): 19.96. MS (ESI) m/z calculated for C₁₇H₃₀N₂O₉PSi⁻ 465.4913, found: 465.23.

### Step 3: (2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl methyl ((((2R,3S,4R,5R)-3-((tert-butyldimethylsilyl)oxy)-5-(2,4-dioxo-3,4-dihydropyrimidin-1(2H)-yl)-4-methoxytetrahydrofuran-2-yl)oxy)methyl)phosphonate (I-1)

4.65 g of 1.3 was dissolved in 100 mL anhydrous pyridine. The mixture was cooled to 0° C. and 7.72 g of TIBSCl (3 eq) was added with stirring for 5 min, followed by warming to rt and stirring for a further 15 min. 4.63 g of 1-((2R,4R,5R)-5-((bis(4-methoxyphenyl)(phenyl)methoxy)methyl)-4-hydroxytetrahydrofuran-2-yl)-5-methylpyrimidine-2,4(1H,3H)-dione was then added to the resulting solution followed by 4.1 mL of 1-methylimidazole. The reaction mixture was stirred for 2 hours at rt. UPLC verified reaction completion and 25 mL saturated sodium bicarbonate was added to quench the reaction. Volatiles were removed under reduced pressure and the resulting yellow/brown oil was purified by flash chromatography (0-10% MeOH in DCM with 0.1% TEA) to afford I-1 (3.67 g, 44% yield) as white powder. MS (ESI) m/z calculated for C₄₈H₆₁N₄NaO₁₅PSi⁺ 1016.0770, found: 1016.01.

### Step 4: (2R,3S,5R)-2-((Bis(4-methoxyphenyl)(phenyl)methoxy)methyl)-5-(5-methyl-2,4-dioxo-3,4-dihydropyrimidin-1(2H)-yl)tetrahydrofuran-3-yl methyl ((((2R,3S,4R,5R)-5-(2,4-dioxo-3,4-dihydropyrimidin-1(2H)-yl)-3-hydroxy-4-methoxytetrahydrofuran-2-yl)oxy)methyl)phosphonate (I-2)

2.50 g of I-1 was dissolved in 50 mL anhydrous THF. Then to the solution was added 7.5 mL of TBAF (1M) dropwise over 3 min with stirring at rt. The resulting solution was kept stirring at rt for 30 min. The reaction was stopped when UPLC indicated >85% of starting material was consumed. Volatiles were removed under reduced pressure to give a yellow oil that was purified by flash chromatography (0-10% MeOH in DCM with 0.1% TEA) to afford I-2 (1.07 g, 49% yield) as white powder. ³¹P-NMR (200 MHz, CDCl₃): 21.93, 21.91. MS (ESI) m/z calculated for C₄₂H₄₆N₄O₁₅P⁻ 877.8173, found: 877.91.

### Step 5: (2R,3S,4R,5R)-2-(((((2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl)oxy)(methoxy)phosphoryl)methoxy)-5-(2,4-dioxo-3,4-dihydropyrimidin-1(2H)-yl)-4-methoxytetrahydrofuran-3-yl (2-cyanoethyl) diisopropylphosphoramidite (I-3)

440 mg compound I-2 was dissolved in 6 mL anhydrous DCM. After stirring at rt for 10 min, 262 μL of 2-cyanoethyl N′,N′,N′,N′-tetraisopropylphosphorodiamidite was added to the reaction mixture followed by 100 mg of 4,5-dicyanoimidazole. The resulting clear solution was kept stirring at rt for 4 hours. UPLC monitoring verified the full conversion of starting material. The reaction mixture was then washed with 5 mL saturated sodium bicarbonate and 5 mL brine. After drying over anhydrous sodium sulfate, the volatiles were removed under reduced pressure. The white oil residue was then purified by flash chromatography (0-8% MeOH in DCM with 0.1% TEA) to afford I-3 (346 mg, 64% yield) as a white powder. ³¹P-NMR (200 MHz, CDCl₃): 152.80, 152.75, 151.53, 151.38, 22.65, 22.47, 22.19, 22.16. MS (ESI) m/z calculated for C₅₁H₆₄N₆NaO₁₆P₂⁺ 1102.0357, found: 1102.08.

### Example 2. Synthesis of (2R,3S,4R,5R)-2-(1-((((2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl)oxy)(methoxy)phosphoryl)ethoxy)-5-(2,4-dioxo-3,4-dihydropyrimidin-1(2H)-yl)-4-methoxytetrahydrofuran-3-yl (2-cyanoethyl) diisopropylphosphoramidite (I-6)

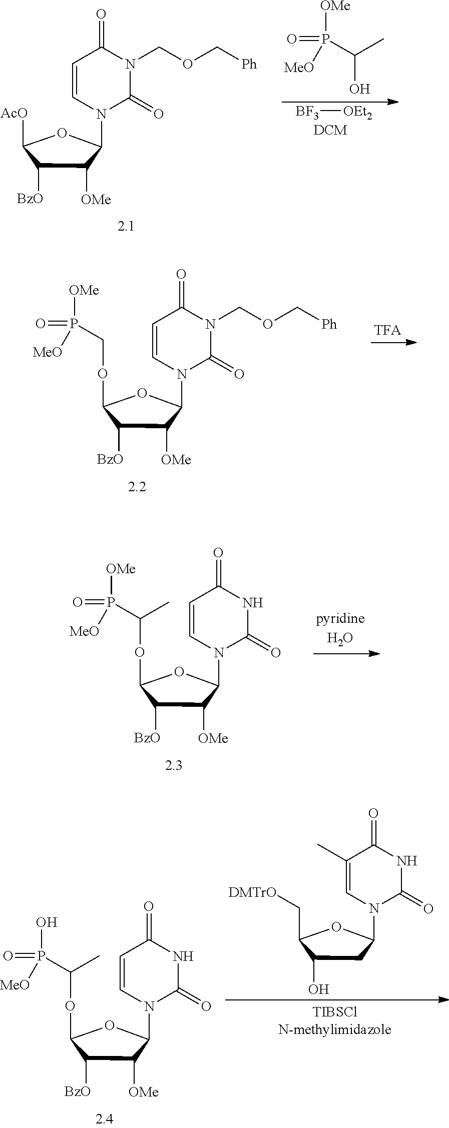

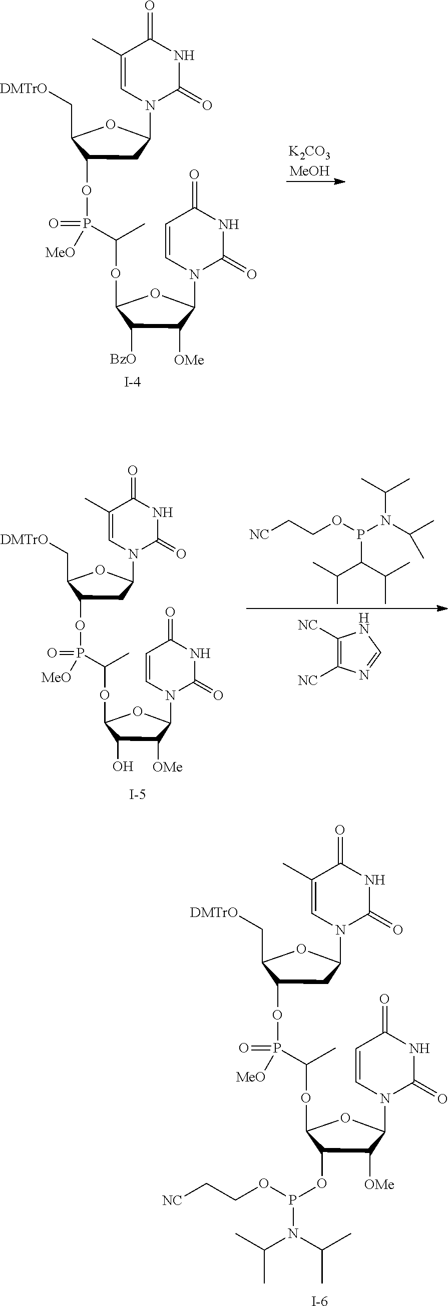

### Step 1: (2R,3S,4R,5R)-5-(3-((Benzyloxy)methyl)-2,4-dioxo-3,4-dihydropyrimidin-1(2H)-yl)-2-((dimethoxyphosphoryl)methoxy)-4-methoxytetrahydrofuran-3-yl benzoate (2.2)

3.06 g of 2.1 was dissolved in 20 mL anhydrous DCM. The solution was then cooled to 0° C. and added 3.77 mL BF₃.Et₂O followed by 3 mL of dimethyl (1-hydroxyethyl)phosphonate. The reaction mixture was stirred for 24 hours at rt before a further 1.9 mL BF₃.Et₂O and 1.5 mL dimethyl (1-hydroxyethyl)phosphonate were added to the reaction. The resulting solution was kept stirring for another 84 hours. After reaction completion as verified by UPLC, the reaction was quenched with 15 mL water and diluted with 40 mL DCM. The layers were separated, and the organic layer was washed with 30 mL of saturated sodium bicarbonate and 30 mL of brine. After drying over anhydrous sodium sulfate, the mixture was concentrated and purified by flash chromatography (30-100% EA in Hexanes followed by 0-5% MeOH in DCM) to afford 2.2 (2.04 g, 56% yield) as a white powder. MS (ESI) m/z calculated for C₂₈H₃₂N₂NaO₁₁P⁺ 627.5380, found: 627.21.

### Step 2: (2R,3S,4R,5R)-2-(1-(Dimethoxyphosphoryl)ethoxy)-5-(2,4-dioxo-3,4-dihydropyrimidin-1(2H)-yl)-4-methoxytetrahydrofuran-3-yl benzoate (2.3)

1.80 g of 2.2 was dissolved in 0.9 mL anhydrous toluene and 7.2 mL of TFA was added to the solution. Then reaction mixture was heated to 45° C. and stirred for 3 hours. After reaction completion as verified by UPLC, the mixture was diluted with 70 mL toluene and volatiles were removed under reduced pressure. The purple/brown residue was then dissolved in 100 mL EA and washed with 50 mL sodium bicarbonate and 50 mL brine. After drying over anhydrous sodium sulfate, volatiles were removed under reduced pressure. Then resulting brown oil was purified by flash chromatography to afford 2.3 (1.11 g, 77% yield) as a white powder. MS (ESI) m/z calculated for C₂₀H₂₅N₂NaO₁₀P⁺ 507.3870, found: 507.43.

### Step 3: (2R,3S,4R,5R)-5-(2,4-dioxo-3,4-dihydropyrimidin-1(2H)-yl)-2-(1-(hydroxy(methoxy)phosphoryl)ethoxy)-4-methoxytetrahydrofuran-3-yl benzoate (2.4)

1.11 g compound 2.3 was dissolved in 40 mL of a 3:2 pyridine and water mixture. The reaction mixture was heated to 50° C. and stirred for 16 hours. After reaction completion as verified by UPLC, the volatiles were removed under reduced pressure and crude 2.4 (1.26 g, quantitative) was used in the next step without further purification.

### Step 4: (2R,3S,4R,5R)-2-(1-((((2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl)oxy)(methoxy)phosphoryl)ethoxy)-5-(2,4-dioxo-3,4-dihydropyrimidin-1(2H)-yl)-4-methoxytetrahydrofuran-3-yl benzoate (I-4)

1.04 g of 2.4 was dissolved in 24 mL anhydrous pyridine and cooled to 0° C. 1.81 g of TIBSCl was added and the mixture was warmed to rt and stirred for 10 min at rt. 2.16 g of 1-((2R,4R,5R)-5-((bis(4-methoxyphenyl)(phenyl)methoxy)methyl)-4-hydroxytetrahydrofuran-2-yl)-5-methylpyrimidine-2,4(1H,3H)-dione was added to the resulting solution followed by 1.0 mL 1-methylimidazole. The reaction mixture was then stirred for 3 hours at rt. After reaction completion as verified by UPLC, 10 mL of saturated sodium bicarbonate was added to quench the reaction. Volatiles are removed under reduced pressure and the resulting yellow oil was purified by flash chromatography (0-10% MeOH in DCM with 0.1% TEA) to afford I-4 (0.89 g, 47% yield) as a white powder. MS (ESI) m/z calculated for C₅₀H₅₃N₄NaO₁₆P⁺ 1019.9490, found: 1019.78.

### Step 5: (2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl methyl (1-(((2R,3S,4R,5R)-5-(2,4-dioxo-3,4-dihydropyrimidin-1(2H)-yl)-3-hydroxy-4-methoxytetrahydrofuran-2-yl)oxy)ethyl)phosphonate (I-5)

1.38 g potassium carbonate was added to 20 mL of MeOH and the resulting slurry was stirred for 12 hours. 0.89 g of I-4 was dissolved in 5 mL anhydrous MeOH and added 5 mL of the pre-made potassium carbonate slurry. The reaction mixture was stirred for 2.5 hours at rt. After reaction completion as verified by UPLC, the reaction mixture was filtered, and the filtrate quenched with 2 mL 1M acetic acid. Volatiles were removed under reduced pressure. The crude was then purified by flash chromatography (30-100% EA in Hexanes followed by 0-5% MeOH in DCM with 0.1% TEA) to afford I-5 (0.42 g, 53% yield) as a white foam. MS (ESI) m/z calculated for C₄₃H₄₉N₄NaO₁₅P⁺ 915.8410, found: 915.48.

### Step 6: (2R,3S,4R,5R)-2-(1-((((2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl)oxy)(methoxy)phosphoryl)ethoxy)-5-(2,4-dioxo-3,4-dihydropyrimidin-1(2H)-yl)-4-methoxytetrahydrofuran-3-yl (2-cyanoethyl) diisopropylphosphoramidite (I-6)

550 mg of I-5 was dissolved in 7.5 mL anhydrous DCM. After stirring at rt for 10 min, 320 μL 2-cyanoethyl N′,N′,N′,N′-tetraisopropylphosphorodiamidite was added to the reaction mixture followed by 90 mg 4,5-dicyanoimidazole. The resulting clear solution was stirred at rt for 3 hours. After reaction completion as verified by UPLC, the reaction mixture was washed with 5 mL saturated sodium bicarbonate and 5 mL brine. After drying over anhydrous sodium sulfate the volatiles were removed under reduced pressure. The white oil residue was then purified by flash chromatography (0-10% MeOH in DCM with 0.1% TEA) to afford I-6 (451 mg, 67% yield) as a white powder. ³¹P-NMR (200 MHz, CDCl₃): 152.48, 152.46, 151.32, 151.29, 24.74, 24.50, 24.13, 23.96. MS (ESI) m/z calculated for C₅₂H₆₆N₆NaO₁₆P₂⁺ 1116.0627, found: 1116.12.

### Example 3. Synthesis of (2R,3R,4S,5R)-2-(((((2R,4S,5R)-5-(6-benzamido-9H-purin-9-yl)-2-((bis(4-methoxyphenyl)(phenyl)methoxy)methyl)-4-methoxytetrahydrofuran-3-yl)oxy)(methoxy)phosphoryl)methoxy)-5-(2,4-dioxo-3,4-dihydropyrimidin-1(2H)-yl)-4-methoxytetrahydrofuran-3-yl (2-cyanoethyl) diisopropylphosphoramidite (I-9)

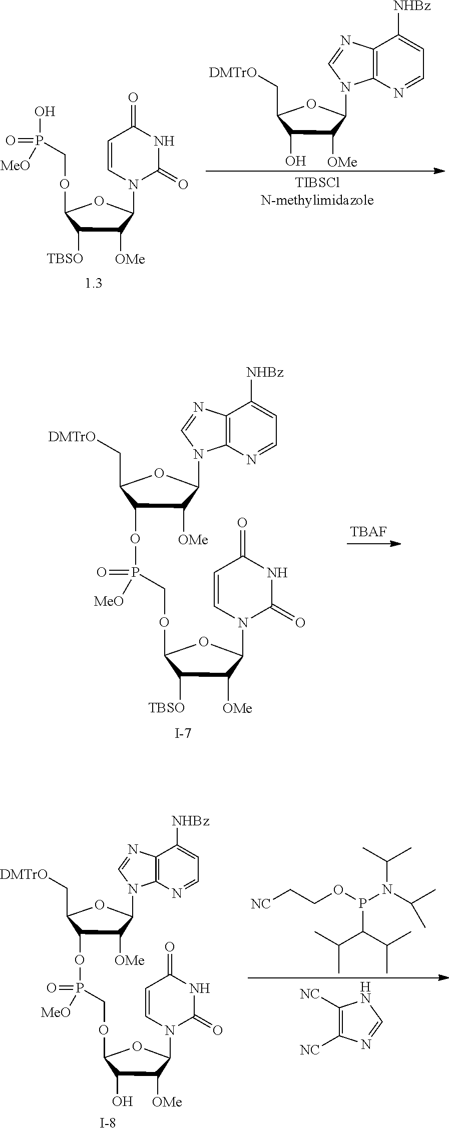

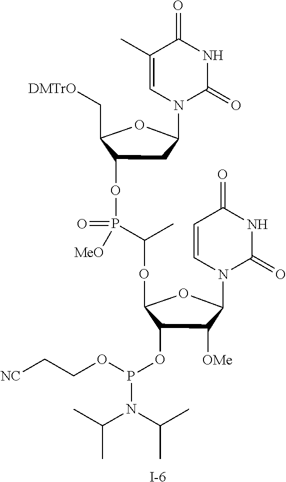

### Step 3: (2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl methyl ((((2R,3S,4R,5R)-3-((tert-butyldimethylsilyl)oxy)-5-(2,4-dioxo-3,4-dihydropyrimidin-1(2H)-yl)-4-methoxytetrahydrofuran-2-yl)oxy)methyl)phosphonate (I-7)

2.60 g of 1.3 was dissolved in 55 mL anhydrous pyridine and cooled to 0° C. TIBSCl (3 eq) was added and stirring was maintained for 15 min at 0° C. After warming to rt, 2.48 g N-(9-((2R,3S,4S,5R)-5-((bis(4-methoxyphenyl)(phenyl)methoxy)methyl)-4-hydroxy-3-methoxytetrahydrofuran-2-yl)-9H-purin-6-yl)benzamide was added followed by 2.3 mL of 1-methylimidazole. The reaction mixture was then stirred for 3 hours at rt. After reaction completion was verified by UPLC, 10 mL of saturated sodium bicarbonate was added to quench the reaction. Volatiles were removed under reduced pressure and the resulting yellow oil was purified by flash chromatography (0-10% MeOH in DCM with 0.1% TEA) to afford I-7 (2.26 g, 55% yield) as a white powder. MS (ESI) m/z calculated for C₅₆H₆₇N₇O₁₅PSi⁺ 1137.2442, found: 1137.45.

### Step 4: (2R,3S,5R)-2-((Bis(4-methoxyphenyl)(phenyl)methoxy)methyl)-5-(5-methyl-2,4-dioxo-3,4-dihydropyrimidin-1(2H)-yl)tetrahydrofuran-3-yl methyl ((((2R,3S,4R,5R)-5-(2,4-dioxo-3,4-dihydropyrimidin-1(2H)-yl)-3-hydroxy-4-methoxytetrahydrofuran-2-yl)oxy)methyl)phosphonate (I-8)

1.30 g of I-7 was dissolved in 40 mL of anhydrous THF and 3.0 mL of TBAF (1M) was added dropwise over 3 min at rt with stirring and the resulting solution was stirred at rt for 1 hour. When >90% of starting material was consumed as verified by UPLC, the volatiles were removed under reduced pressure to give a yellow oil that was purified by flash chromatography (0-10% MeOH in DCM with 0.1% TEA) to afford I-8 (0.53 g, 45% yield) as off-white powder. MS (ESI) m/z calculated for C₅₀H₅₁N₇O₁₅P⁻ 1021.9663, found: 1020.84.

### Step 5: (2R,3S,4R,5R)-2-(((((2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl)oxy)(methoxy)phosphoryl)methoxy)-5-(2,4-dioxo-3,4-dihydropyrimidin-1(2H)-yl)-4-methoxytetrahydrofuran-3-yl (2-cyanoethyl) diisopropylphosphoramidite (I-9)

416 mg of I-8 was dissolved in 5 mL anhydrous DCM. After stirring at rt for 10 min, 224 μL of 2-cyanoethyl N′,N′,N′,N′-tetraisopropylphosphorodiamidite was added to the reaction mixture followed by 63 mg of 4,5-dicyanoimidazole. The resulting clear solution was stirred at rt for 3 hours. After reaction completion as verified by UPLC, the reaction mixture was washed with 5 mL saturated sodium bicarbonate, 5 mL brine, dried over anhydrous sodium sulfate, and concentrated under reduced pressure. The resulting white oil residue was then purified by flash chromatography (0-10% MeOH in DCM with 0.1% TEA) to afford I-9 (305 mg, 61% yield) as a white foam. ³¹P-NMR (200 MHz, CDCl₃): 152.89, 152.71, 151.60, 151.51, 23.19, 22.81, 22.16, 21.75. MS (ESI) m/z calculated for C₅₉H₇₀N₉O₁₆P₂⁺ 1223.2023, found: 1222.91.

### Example 4. Synthesis of (2R,3R,5R)-2-(((((2R,3R,5R)-5-(6-benzamido-9H-purin-9-yl)-2-((bis(4-methoxyphenyl)(phenyl)methoxy)methyl)tetrahydrofuran-3-yl)oxy)(methoxy)phosphoryl)methoxy)-5-(5-methyl-2,4-dioxo-3,4-dihydropyrimidin-1(2H)-yl)tetrahydrofuran-3-yl (2-cyanoethyl) diisopropylphosphoramidite (I-12)

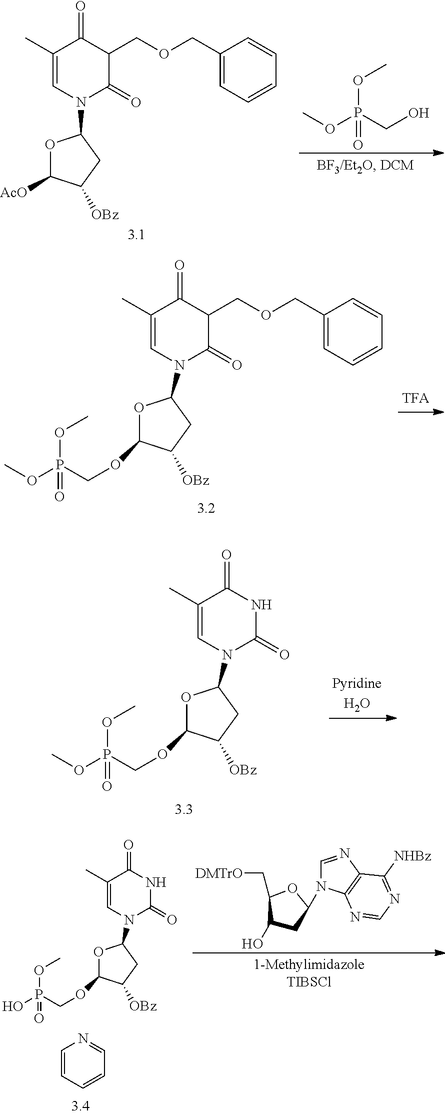

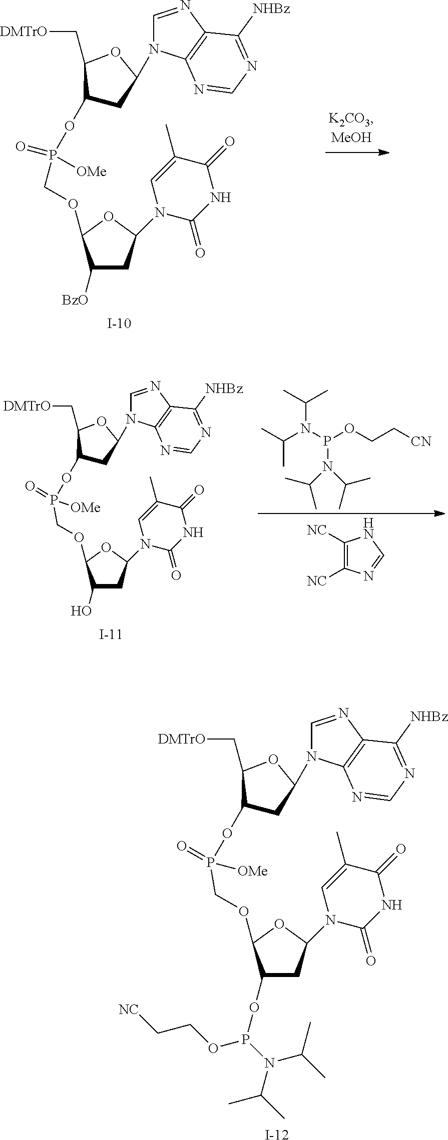

### Step 1: (2R,3S,5R)-5-(3-((benzyloxy)methyl)-5-methyl-2,4-dioxo-3,4-dihydropyrimidin-1(2H)-yl)-2-(1-(dimethoxyphosphoryl)ethoxy)tetrahydrofuran-3-yl benzoate (3.2)

4.10 g of 3.1 was dissolved in 24 mL anhydrous DCM. The solution was then cooled to 0° C. and added 5.40 mL BF₃.Et₂O followed by 3.60 mL of dimethyl (hydroxymethyl)phosphonate. The reaction mixture was stirred under 0° C. for 15 min and allowed to warm to rt gradually. The resulting solution was stirred for 18 hours at rt. After reaction completion as verified by UPLC, the reaction was quenched with 20 mL water and diluted with 60 mL DCM. The layers were separated, and the organic layer was washed with 30 mL of saturated sodium bicarbonate and 30 mL of brine. After drying over anhydrous sodium sulfate, the mixture was concentrated and purified by flash chromatography (30-100% EA in Hexanes followed by 0-10% MeOH in DCM) to afford 3.2 (2.15 g, 45% yield) as a white powder. 1H-NMR (400 MHz, CDCl3) δ (ppm): 8.04 (dd, J1=12 Hz, J2=4 Hz, 2H), 7.24-7.64 (m, 9H), 6.86 (t, J=8 Hz, 1H), 5.53 (s, 1H), 5.52 (s, 2H), 5.21 (s, 1H), 4.72 (s, 2H), 4.06 (dd, J1=14 Hz, J2=8 Hz, 1H), 3.82-3.90 (m, 7H), 2.58-2.63 (m, 1H), 2.32-2.39 (m, 1H), 2.02 (s, 3H). MS (ESI) m/z calculated for C₂₇H₃₁N₂NaO₁₀P⁺ 597.1614, found: 597.1783.

### Step 2: (2R,3S,5R)-2-((dimethoxyphosphoryl)methoxy)-5-(5-methyl-2,4-dioxo-3,4-dihydropyrimidin-1(2H)-yl) tetrahydrofuran-3-yl benzoate. (3.3)

2.12 g compound 3.2 was dissolved in 2.0 mL anhydrous toluene and 16.0 mL of TFA was added to the solution. Then reaction mixture was heated to 45° C. and stirred for 5 hours. After reaction completion as verified by UPLC, the mixture was diluted with 70 mL toluene and volatiles were removed under reduced pressure. The purple/brown residue was then dissolved in 100 mL EA and washed with 100 mL sodium bicarbonate and 100 mL brine. After drying over anhydrous sodium sulfate, volatiles were removed under reduced pressure. Then resulting brown oil was purified by flash chromatography to afford 3.3 (1.21 g, 75% yield) as a white powder. 1H-NMR (400 MHz, CDCl3) δ (ppm): 8.46 (s, 1H), 7.40-8.05 (m, 6H), 6.82 (t, J=8 Hz, 1H), 5.53 (s, 1H), 5.22 (s, 1H), 4.07 (dd, J1=14 Hz, J2=8 Hz, 1H), 3.83-3.91 (m, 7H), 2.59-2.64 (m, 1H), 2.38-2.44 (m, 1H), 1.98 (s, 3H). MS (ESI) m/z calculated for C₁₉H₂₃N₂NaO₉P⁺ 477.3615, found: 477.4387.

### Step 3: (2R,3S,5R)-2-((hydroxy(methoxy)phosphoryl)methoxy)-5-(5-methyl-2,4-dioxo-3,4-dihydropyrimidin-1(2H)-yl)tetrahydrofuran-3-yl benzoate (3.4)

1.2 g compound 3.4 was dissolved in 40 mL of a 3:2 pyridine and water mixture. The reaction mixture was heated to 50° C. and stirred for 16 hours. After reaction completion as verified by UPLC, the volatiles were removed under reduced pressure and crude 3.4 (1.37 g, quantitative) was used in the next step without further purification.

### Step 4: (2R,3R,5R)-2-(((((2R,3R,5R)-5-(6-benzamido-9H-purin-9-yl)-2-((bis(4-methoxyphenyl)(phenyl)methoxy)methyl)tetrahydrofuran-3-yl)oxy)(methoxy)phosphoryl)methoxy)-5-(5-methyl-2,4-dioxo-3,4-dihydropyrimidin-1(2H)-yl)tetrahydrofuran-3-yl benzoate (I-10)

2.56 g of 3.4 and 2.98 g of 1-((2R,4R,5R)-5-((bis(4-methoxyphenyl)(phenyl)methoxy)methyl)-4-hydroxytetrahydrofuran-2-yl)-5-methylpyrimidine-2,4(1H,3H)-dione were dissolved in 64 mL anhydrous pyridine and cooled to 0° C. 3.44 g of TIBSCl was added. The mixture was then stirred for 15 min 0° C. After warmed to rt, reaction mixture was added 2.5 mL 1-methylimidazole. The reaction mixture was then stirred for 3.5 hours at rt. After reaction completion as verified by UPLC, 30 mL of saturated sodium bicarbonate was added to quench the reaction. Volatiles are removed under reduced pressure and the resulting yellow oil was purified by flash chromatography (0-10% MeOH in EA with 0.1% TEA) to afford I-10 (2.42 g, 46% yield) as a white powder. MS (ESI) m/z calculated for C₅₆H₅₅N₇O₁₄P⁺ 1080.3545, found: 1080.3895.

### Step 5: (2R,3R,5R)-5-(6-benzamido-9H-purin-9-yl)-2-((bis(4-methoxyphenyl) (phenyl)methoxy)methyl)tetrahydrofuran-3-yl methyl ((((2R,3R,5R)-3-hydroxy-5-(5-methyl-2,4-dioxo-3,4-dihydropyrimidin-1(2H)-yl)tetrahydrofuran-2-yl)oxy)methyl)phosphonate (I-11)

1.38 g potassium carbonate was added to 20 mL of MeOH and the resulting slurry was stirred for 12 hours. The slurry was then filtered to result clear filtrate as potassium carbonate solution. 1.0 g of I-10 was dissolved in 45 mL anhydrous MeOH and added 5 mL of the pre-made potassium carbonate solution. The reaction mixture was stirred for 1 hours at rt. After reaction completion as verified by UPLC, the reaction mixture was filtered, and the filtrate quenched with 1.5 mL 1M acetic acid. Volatiles were removed under reduced pressure. The crude was then purified by flash chromatography (30-100% EA in Hexanes with 0.1% TEA followed by 0-10% MeOH in DCM) to afford I-11 (0.52 g, 58% yield) as a white foam. MS (ESI) m/z calculated for C₄₉H₅₁N₇O₁₃P⁺ 976.3283, found: 976.6138.

### Step 6: (2R,3R,5R)-2-(((((2R,3R,5R)-5-(6-benzamido-9H-purin-9-yl)-2-((bis(4-methoxyphenyl)(phenyl)methoxy)methyl)tetrahydrofuran-3-yl)oxy)(methoxy)phosphoryl)methoxy)-5-(5-methyl-2,4-dioxo-3,4-dihydropyrimidin-1(2H)-yl)tetrahydrofuran-3-yl (2-cyanoethyl) diisopropylphosphoramidite (I-12)

291 mg of I-11 was dissolved in 4.0 mL anhydrous DCM. After stirring at rt for 10 min, 143 μL 2-cyanoethyl N′,N′,N′,N′-tetraisopropylphosphorodiamidite was added to the reaction mixture followed by 39 mg 4,5-dicyanoimidazole. The resulting clear solution was stirred at rt for 3 hours. After reaction completion as verified by UPLC, the reaction mixture was washed with 5 mL saturated sodium bicarbonate and 5 mL brine. After drying over anhydrous sodium sulfate the volatiles were removed under reduced pressure. The white oil residue was then purified by flash chromatography (0-10% MeOH in DCM with 0.1% TEA) to afford I-12 (220 mg, 62% yield) as a white powder. 31P-NMR (200 MHz, DMSO-d6): 149.64, 149.51, 149.49, 149.37, 23.67, 23.65, 23.38, 23.33. MS (ESI) m/z calculated for C₅₈H₆₈N₉O₁₄P₂⁺ 1176.4361, found: 1176.7185.

### Example 5. Synthesis of (2R,3R,5R)-2-(1-((((2R,3R,5R)-5-(6-benzamido-9H-purin-9-yl)-2-((bis(4-methoxyphenyl)(phenyl)methoxy)methyl)tetrahydrofuran-3-yl)oxy)(methoxy)phosphoryl)ethoxy)-5-(5-methyl-2,4-dioxo-3,4-dihydropyrimidin-1(2H)-yl)tetrahydrofuran-3-yl (2-cyanoethyl) diisopropylphosphoramidite (I-15)

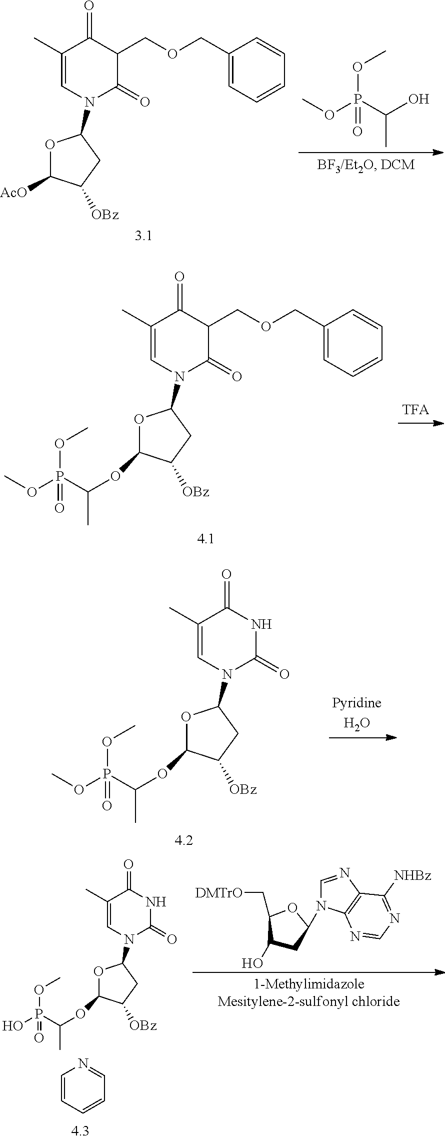

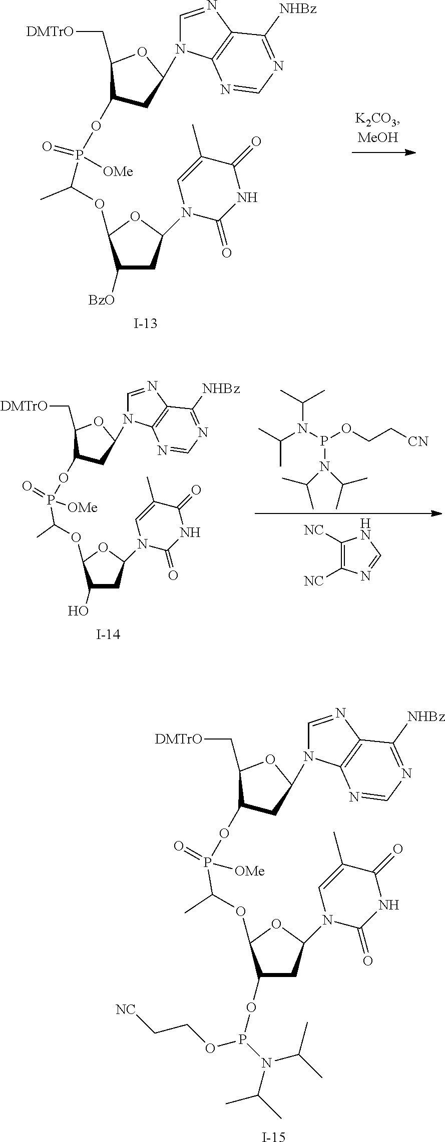

### Step 1: (2R,3S,5R)-5-(3-((benzyloxy)methyl)-5-methyl-2,4-dioxo-3,4-dihydropyrimidin-1(2H)-yl)-2-(1-(dimethoxyphosphoryl)ethoxy)tetrahydrofuran-3-yl benzoate (4.1)

4.50 g of 3.1 was dissolved in 27 mL anhydrous DCM. The solution was then cooled to 0° C. and added 5.90 mL BF₃.Et₂O followed by 4.50 mL of dimethyl (1-hydroxyethyl)phosphonate. The reaction mixture was stirred under 0° C. for 15 min and allowed to warm to rt gradually. The resulting solution was stirred for 24 hours at rt. After reaction completion as verified by UPLC, the reaction was quenched with 40 mL water and diluted with 100 mL DCM. The layers were separated, and the organic layer was washed with 30 mL of saturated sodium bicarbonate and 30 mL of brine. After drying over anhydrous sodium sulfate, the mixture was concentrated and purified by flash chromatography (30-100% EA in Hexanes followed by 0-10% MeOH in DCM) to afford 4.1 (2.15 g, 45% yield) as a white powder. MS (ESI) m/z calculated for C₂₈H₃₃N₂NaO₁₀P⁺ 611.1771, found: 611.3412.

### Step 2: (2R,3S,5R)-2-(1-(dimethoxyphosphoryl)ethoxy)-5-(5-methyl-2,4-dioxo-3,4-dihydropyrimidin-1(2H)-yl)tetrahydrofuran-3-yl benzoate. (4.2)

1.90 g compound 4.1 was dissolved in 2.0 mL anhydrous toluene and 16.0 mL of TFA was added to the solution. Then reaction mixture was heated to 45° C. and stirred for 5 hours. After reaction completion as verified by UPLC, the mixture was diluted with 70 mL toluene and volatiles were removed under reduced pressure. The purple/brown residue was then dissolved in 100 mL EA and washed with 100 mL sodium bicarbonate and 100 mL brine. After drying over anhydrous sodium sulfate, volatiles were removed under reduced pressure. Then resulting brown oil was purified by flash chromatography to afford 4.2 (1.15 g, 76% yield) as a white powder. MS (ESI) m/z calculated for C₂₀H₂₅N₂NaO₉P⁺ 491.1195, found: 491.2625.

### Step 3: (2R,3S,5R)-2-(1-(hydroxy(methoxy)phosphoryl)ethoxy)-5-(5-methyl-2,4-dioxo-3,4-dihydropyrimidin-1(2H)-yl)tetrahydrofuran-3-yl benzoate (4.3)

1.0 g compound 4.2 was dissolved in 40 mL of a 3:2 pyridine and water mixture. The reaction mixture was heated to 50° C. and stirred for 16 hours. After reaction completion as verified by UPLC, the volatiles were removed under reduced pressure and crude 4.3 (1.14 g, quantitative) was used in the next step without further purification.

### Step 4: (2R,3R,5R)-2-(1-((((2R,3R,5R)-5-(6-benzamido-9H-purin-9-yl)-2-((bis(4-methoxyphenyl)(phenyl)methoxy)methyl)tetrahydrofuran-3-yl)oxy)(methoxy)phosphoryl)ethoxy)-5-(5-methyl-2,4-dioxo-3,4-dihydropyrimidin-1(2H)-yl)tetrahydrofuran-3-yl benzoate (I-13)

1.35 g of 4.3 and 1.50 g of 1-((2R,4R,5R)-5-((bis(4-methoxyphenyl) (phenyl)methoxy)methyl)-4-hydroxytetrahydrofuran-2-yl)-5-methylpyrimidine-2,4(1H,3H)-dione were dissolved in 27 mL anhydrous pyridine and cooled to 0° C. 2.35 g of Mesitylene-2-sulfonyl chloride was added. The mixture was then stirred for 15 min 0° C. After warmed to rt, reaction mixture was added 1.20 mL 1-methylimidazole. The reaction mixture was then stirred for 4 hours at rt. After reaction completion as verified by UPLC, 30 mL of saturated sodium bicarbonate was added to quench the reaction. Volatiles are removed under reduced pressure and the resulting yellow oil was purified by flash chromatography (0-10% MeOH in EA with 0.1% TEA) to afford I-13 (1.03 g, 46% yield) as a white powder. MS (ESI) m/z calculated for C₅₇H₅₇N₇O₁₄P⁺ 1094.3701, found: 1094.3352.

### Step 5: (2R,3R,5R)-5-(6-benzamido-9H-purin-9-yl)-2-((bis(4-methoxyphenyl) (phenyl)methoxy)methyl)tetrahydrofuran-3-yl methyl (1-(((2R,3R,5R)-3-hydroxy-5-(5-methyl-2,4-dioxo-3,4-dihydropyrimidin-1(2H)-yl)tetrahydrofuran-2-yl)oxy)ethyl)phosphonate (I-14)

1.38 g potassium carbonate was added to 20 mL of MeOH and the resulting slurry was stirred for 12 hours. The slurry was then filtered to result clear filtrate as potassium carbonate solution. 680 mg of 1-13 was dissolved in 45 mL anhydrous MeOH and added 5 mL of the pre-made potassium carbonate solution. The reaction mixture was stirred for 1.5 hours at rt. After reaction completion as verified by UPLC, the reaction mixture was filtered, and the filtrate quenched with 1.5 mL 1M acetic acid. Volatiles were removed under reduced pressure. The crude was then purified by flash chromatography (30-100% EA in Hexanes with 0.1% TEA followed by 0-10% MeOH in DCM) to afford I-14 (310 mg, 51% yield) as a white foam. MS (ESI) m/z calculated for C₅₀H₅₃N₇O₁₃P⁺ 990.3439, found: 990.5858.

### Step 6: (2R,3R,5R)-2-(1-((((2R,3R,5R)-5-(6-benzamido-9H-purin-9-yl)-2-((bis(4-methoxyphenyl)(phenyl)methoxy)methyl)tetrahydrofuran-3-yl)oxy)(methoxy)phosphoryl)ethoxy)-5-(5-methyl-2,4-dioxo-3,4-dihydropyrimidin-1(2H)-yl)tetrahydrofuran-3-yl (2-cyanoethyl) diisopropylphosphoramidite (I-15)

300 mg of I-14 was dissolved in 4.0 mL anhydrous DCM. After stirring at rt for 10 min, 150 μL 2-cyanoethyl N′,N′,N′,N′-tetraisopropylphosphorodiamidite was added to the reaction mixture followed by 42 mg 4,5-dicyanoimidazole. The resulting clear solution was stirred at rt for 3 hours. After reaction completion as verified by UPLC, the reaction mixture was washed with 5 mL saturated sodium bicarbonate and 5 mL brine. After drying over anhydrous sodium sulfate the volatiles were removed under reduced pressure. The white oil residue was then purified by flash chromatography (0-10% MeOH in DCM with 0.1% TEA) to afford I-15 (225 mg, 74% yield) as a white powder. 31P-NMR (200 MHz, DMSO-d6): 149.41, 148.96, 148.84, 148.74, 148.73, 148.66, 148.61, 148.53, 24.61, 24.58, 24.55, 24.52, 24.48, 24.46, 24.42, 24.37. MS (ESI) m/z calculated for C₅₉H₇₀N₉O₁₄P₂⁺ 1190.4518, found: 1190.4528.

### Example 6. Synthesis of (2R,3S,5R)-2-(((((2R,3R,5R)-5-(4-benzamido-5-methyl-2-oxopyrimidin-1(2H)-yl)-2-((bis(4-methoxyphenyl)(phenyl)methoxy)methyl)tetrahydrofuran-3-yl)oxy)(methoxy)phosphoryl)methoxy)-5-(6-benzamido-9H-purin-9-yl)tetrahydrofuran-3-yl (2-cyanoethyl) diisopropylphosphoramidite (I-18)

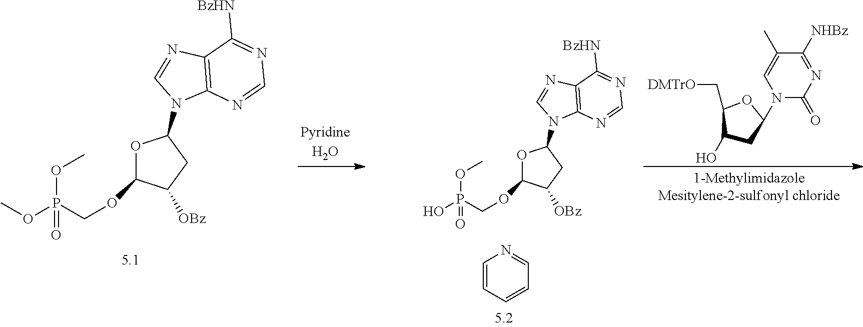

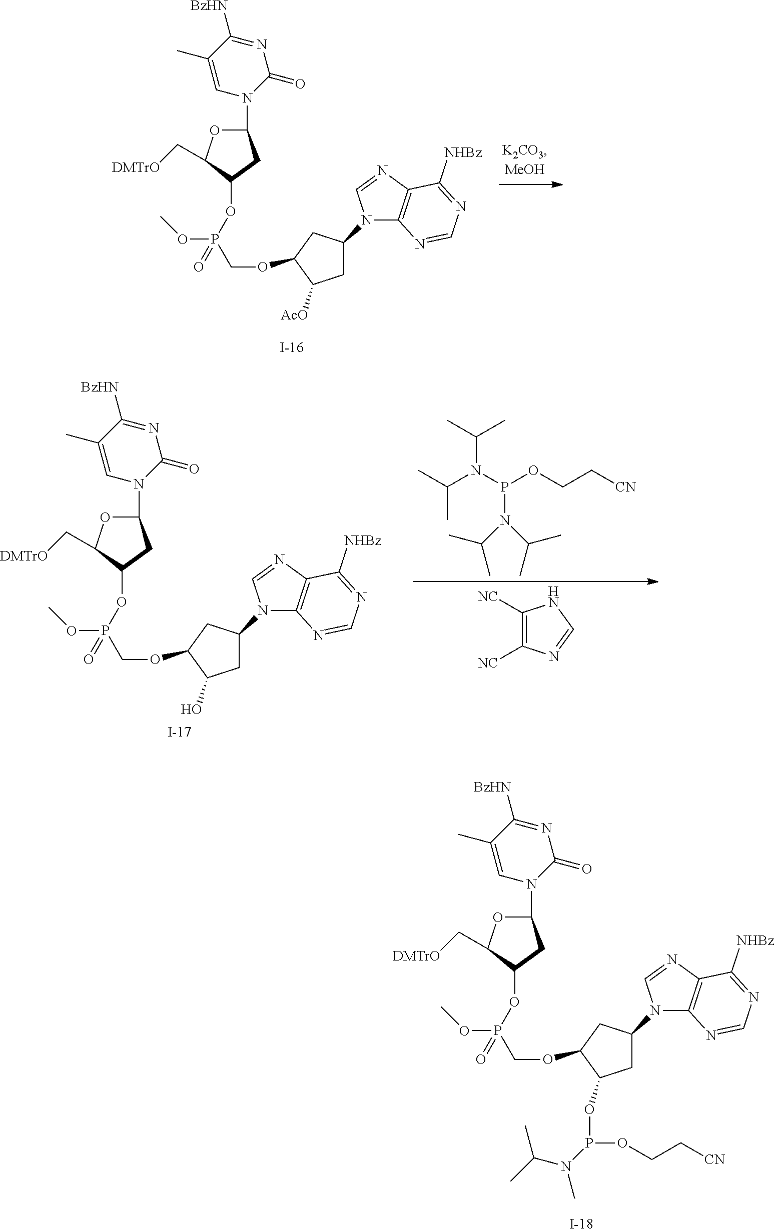

### Step 1: (2R,3S,5R)-5-(6-benzamido-9H-purin-9-yl)-2-((hydroxy(methoxy) phosphoryl)methoxy)tetrahydrofuran-3-yl acetate (5.2)

2.0 g compound 5.1 ((2R,3S,5R)-5-(6-benzamido-9H-purin-9-yl)-2-((dimethoxyphosphoryl)methoxy)tetrahydrofuran-3-yl acetate) was dissolved in 72 mL of a 5:4 pyridine and water mixture. The reaction mixture was heated to 50° C. and stirred for 16 hours. After reaction completion as verified by UPLC, the volatiles were removed under reduced pressure and crude 5.2 (2.26 g, quantitative) was used in the next step without further purification.

### Step 2: (2R,3R,5R)-2-(((((2R,3R,5R)-5-(6-benzamido-9H-purin-9-yl)-2-((bis(4-methoxyphenyl)(phenyl)methoxy)methyl)tetrahydrofuran-3-yl)oxy)(methoxy)phosphoryl)methoxy)-5-(5-methyl-2,4-dioxo-3,4-dihydropyrimidin-1(2H)-yl)tetrahydrofuran-3-yl benzoate (I-16)

1.87 g of 5.2 and 2.02 g of N-(1-((2R,4R,5R)-5-((bis(4-methoxyphenyl) (phenyl)methoxy)methyl)-4-hydroxytetrahydrofuran-2-yl)-5-methyl-2-oxo-1,2-dihydropyrimidin-4-yl)benzamide were dissolved in 47.5 mL anhydrous pyridine and cooled to 0° C. 3.44 g of Mesitylene-2-sulfonyl chloride was added. The mixture was then stirred for 15 min 0° C. After warmed to rt, reaction mixture was added 1.6 mL 1-methylimidazole. The reaction mixture was then stirred for 3 hours at rt. After reaction completion as verified by UPLC, 30 mL of saturated sodium bicarbonate was added to quench the reaction. Volatiles are removed under reduced pressure and the resulting yellow oil was purified by flash chromatography (0-10% MeOH in EA with 0.1% TEA) to afford I-16 (1.72 g, 46% yield) as a white powder. MS (ESI) m/z calculated for C₅₈H₅₈N₈O₁₄P⁺ 1121.3810, found: 1121.4519.

### Step 5: (2R,3R,5R)-5-(4-benzamido-5-methyl-2-oxopyrimidin-1(2H)-yl)-2-((bis(4-methoxyphenyl)(phenyl)methoxy)methyl)tetrahydrofuran-3-yl methyl ((((2R,3S,5R)-5-(6-benzamido-9H-purin-9-yl)-3-hydroxytetrahydrofuran-2-yl)oxy)methyl)phosphonate (I-17)

1.38 g potassium carbonate was added to 20 mL of MeOH and the resulting slurry was stirred for 12 hours. The slurry was then filtered to result clear filtrate as potassium carbonate solution. 1.4 g of I-16 was dissolved in 100 mL anhydrous MeOH and added 4 mL of the pre-made potassium carbonate solution. The reaction mixture was stirred for 20 minutes at rt. After reaction completion as verified by UPLC, the reaction mixture was filtered, and the filtrate quenched with 1.5 mL 1M acetic acid. Volatiles were removed under reduced pressure. The crude was then purified by flash chromatography (30-100% EA in Hexanes with 0.1% TEA followed by 0-10% MeOH in DCM) to afford I-17 (0.85 g, 63% yield) as a white foam. MS (ESI) m/z calculated for C₅₆H₅₆N₈O₁₃P⁺ 1079.3705, found: 1079.6301.

### Step 6: (2R,3S,5R)-2-(((((2R,3R,5R)-5-(4-benzamido-5-methyl-2-oxopyrimidin-1(2H)-yl)-2-((bis(4-methoxyphenyl)(phenyl)methoxy)methyl)tetrahydrofuran-3-yl)oxy)(methoxy)phosphoryl)methoxy)-5-(6-benzamido-9H-purin-9-yl)tetrahydrofuran-3-yl (2-cyanoethyl) diisopropylphosphoramidite (I-18)

640 mg of I-17 was dissolved in 10.0 mL anhydrous DCM. After stirring at rt for 10 min, 285 μL 2-cyanoethyl N′,N′,N′,N′-tetraisopropylphosphorodiamidite was added to the reaction mixture followed by 99 mg 4,5-dicyanoimidazole. The resulting clear solution was stirred at rt for 3 hours. After reaction completion as verified by UPLC, the reaction mixture was washed with 5 mL saturated sodium bicarbonate and 5 mL brine. After drying over anhydrous sodium sulfate the volatiles were removed under reduced pressure. The white oil residue was then purified by flash chromatography (0-10% MeOH in DCM with 0.1% TEA) to afford I-18 (420 mg, 55% yield) as a white powder. 31P-NMR (200 MHz, DMSO-d6): 149.59, 149.56, 149.47, 149.46, 23.83, 23.82, 23.38, 23.36. MS (ESI) m/z calculated for C₆₅H₇₃N₁₀O₁₅P₂⁺ 1279.4783, found: 1279.7819.

### Example 7. Effect of Replacing Phosphorothioate Linkage with Phosphodiester Linkage in the SGLT2 ASO Backbone

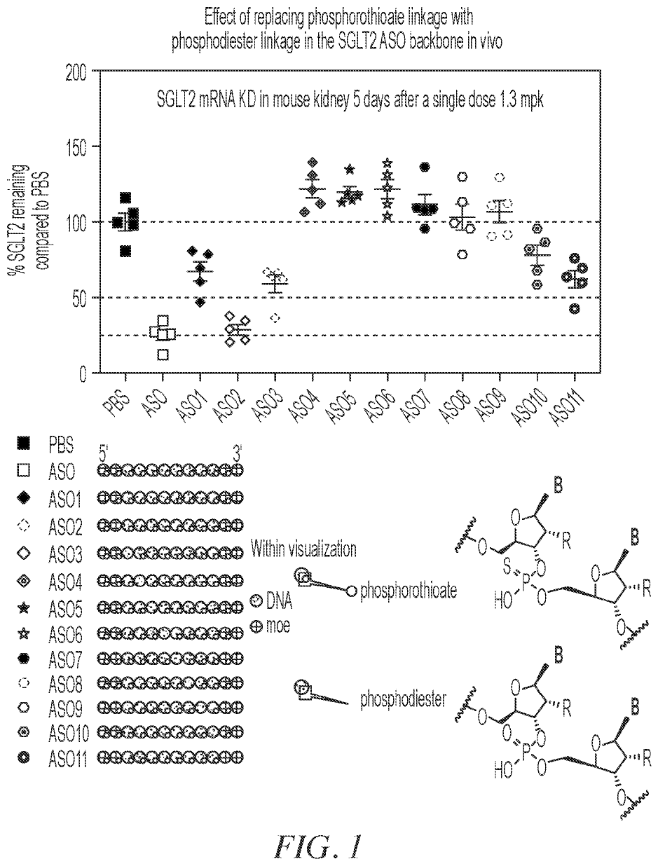

In FIG. 1, ASO is SGLT2 benchmark ASO. ASO1, ASO2, ASO3, ASO4, ASO5, ASO6, ASO7, ASO8, ASO9, ASO10, and ASO11, represent replacing internucleotide phosphorothioate (PS) linkage on benchmark ASO with internucleotide phosphodiester (PO) linkage between nucleotide 1 and 2, 2 and 3, 3 and 4, 4 and 5, 5 and 6, 6 and 7, 7 and 8, 8 and 9, 9 and 10, 10 and 11, 11 and 12 (counting from 5′-end to 3′-end) respectively.

The above oligonucleotides were used to treat female CD-1 IGS mice (aged 6-8 weeks old) subcutaneously using a dose volume of 0.2 ml. The dose administered was 1.3 mg/kg based on RNA weight and formulated in phosphate buffered saline. 5 days later, animals were euthanized by CO₂, and exsanguinated by cardiac puncture. Kidney samples were collected using a 4 mm diameter disposable punch biopsy and fixed for 24 h using RNAlater™ solution. Tissue samples were homogenized in Trizol™ reagent using 5 mm steel beads, and total RNA was isolated using the MagMAX™ system using manufacturer's recommendations. From total RNA, standard industry methodologies were utilized to generate cDNA (single-strand synthesis), and the cDNA was used as the substrate for TaqMan™ quantitative real-time PCR (qRT-PCR) for quantitative detection of SGLT2 mRNA. Relative SGLT2 mRNA was calculated using the standard ddCt method and normalized to Ppib mRNA as a reference gene.

The SGLT2 mRNA knockdown results in FIG. 1 demonstrated that replacing a single PS internucleotide linkage with a PO linkage in the SGLT2 ASO molecule reduces potency significantly at all positions on the backbone except at the position between nucleotide 2 and 3 (ASO2). The reduction of activity was partial when PS was replaced with PO between nucleotide 1 and 2 (ASO1), 3 and 4 (ASO3), as well as 11 and 12 (ASO11). At any other position, replacing PS with PO abolishes the activity.

### Example 8. Effect of Replacing Phosphorothioate Linkage with iMOP in the SGLT2 ASO Backbone

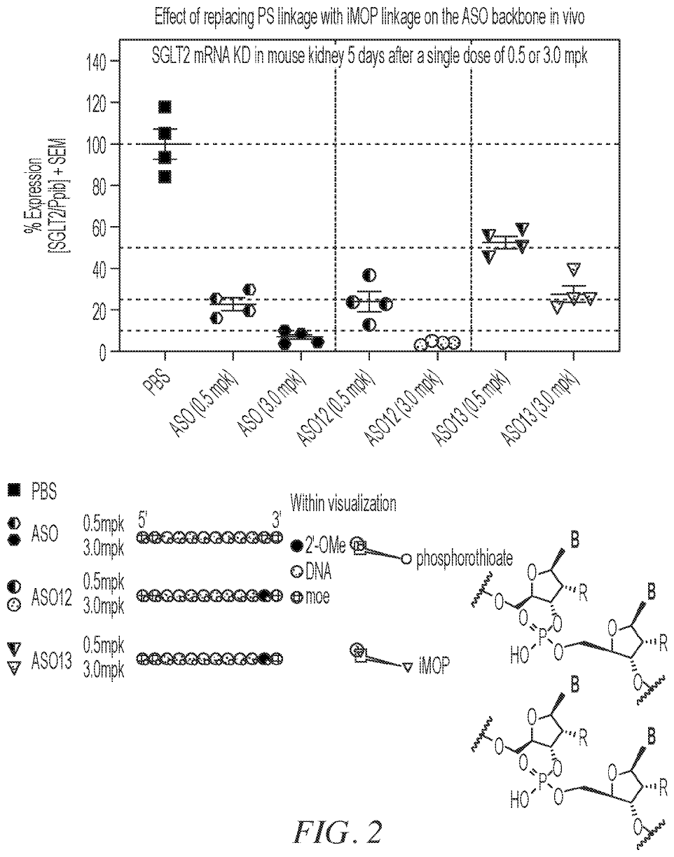

In FIG. 2, ASO is SGLT2 benchmark ASO. ASO12 is an experimental control that the only difference from the benchmark is the 2′-modification of the nucleotide 11 (counting from 5′-end) being 2′-OMe instead of 2′-MOE. ASO13 is a test article of which the linkage between nucleotide 10 and 11 is iMOP (shown in nucleic acid I-3) instead of PS. The rest of ASO13 is identical to ASO12.

The above oligonucleotides were used to treat female CD-1 IGS mice (aged 6-8 weeks old) subcutaneously using a dose volume of 0.2 ml. The dose administered was 0.5 or 3 mg/kg, based on RNA weight and formulated in phosphate buffered saline (PBS). 5 days later, animals were euthanized by CO2, and exsanguinated by cardiac puncture. Kidney samples were collected using a 4 mm diameter disposable punch biopsy and fixed for 24 h using RNAlater™ solution. Tissue samples were homogenized in Trizol™ reagent using 5 mm steel beads, and total RNA was isolated using the MagMAX™ system using manufacturer's recommendations. From total RNA, standard industry methodologies were utilized to generate cDNA (single-strand synthesis), and the cDNA was used as the substrate for TaqMan™ quantitative real-time PCR (qRT-PCR) for quantitative detection of SGLT2 mRNA. Relative SGLT2 mRNA was calculated using the standard ddCt method and normalized to Ppib mRNA as a reference gene.

FIG. 2 demonstrates that replacing a PS linkage with an iMOP linkage in the SGLT2 ASO substantially maintained the in vivo mRNA KD activity with an ED₅₀ of ˜0.5 mpk, in contrast to the phosphodiester replacement shown in FIG. 1. The 2′-OMe experimental control (ASO12) showed equal potency to the benchmark (ASO). All three oligonucleotides ASO, ASO12, and ASO13 showed dose-dependent activity.

### Example 9. Effect of Replacing Phosphorothioate Linkage with iMOP and iMeMOP in the SGLT2 ASO Backbone

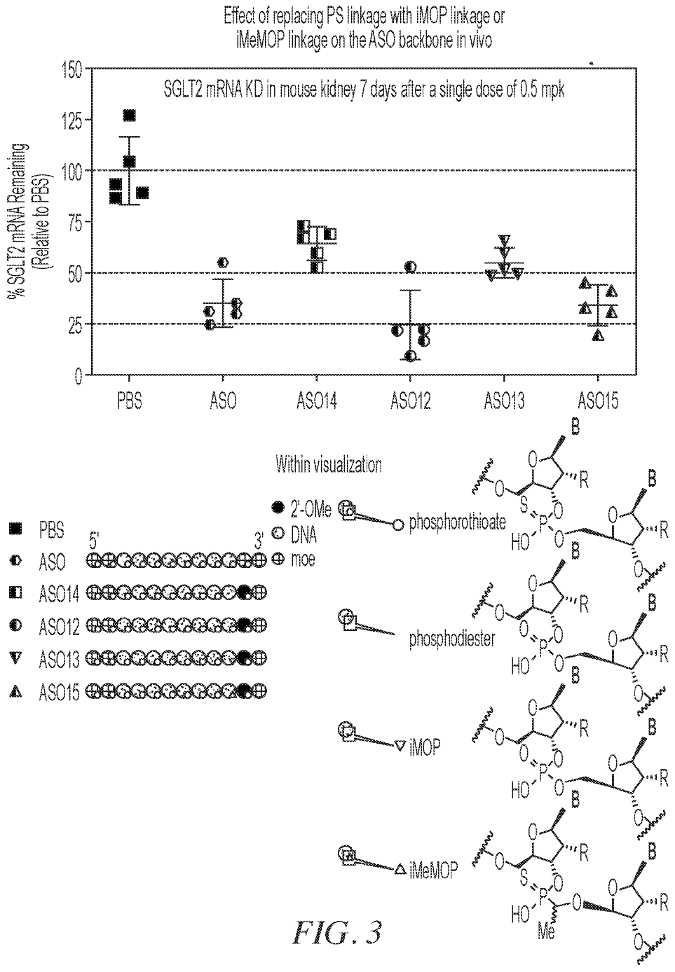

In FIG. 3, ASO is SGLT2 benchmark ASO. ASO14 is a PO control of which the linkage between nucleotide 10 and 11 is a phosphodiester linkage and nucleotide 11 is 2′-OMe. ASO12 is a PS control of which all linkages are PS and nucleotide 11 is 2′-OMe. ASO13 is the iMOP test article of which the linkage between nucleotide 10 and 11 is iMOP (shown in nucleic acid I-3) instead of PS. ASO15 is the iMeMOP test article of which the linkage between nucleotide 10 and 11 is iMeMOP (shown in nucleic acid I-6) instead of PS.

The test articles described above were dissolved in phosphate buffered saline (PBS) and subcutaneously injected into female CD-1 mice at 0.5 mg/kg. Tissue samples were harvested 7 days after PBS or test article injection. Tissue samples were then homogenized in QIAzol Lysis Reagent using TissueLyser II (Qiagen, Valencia, Calif.). RNA was then purified using MagMAX Technology according to manufacturer instructions (ThermoFisher Scientific, Waltham, Mass.). High capacity cDNA reverse transcription kit (ThermoFisher Scientific, Waltham, Mass.) was used to prepare cDNA. Mouse-specific SGLT2 primers (Integrated DNA Technology, Coralville, Iowa) were used for PCR on a CFX384 Real-Time PCR Detection System (Bio-Rad Laboratories, Inc., Hercules, Calif.).

The results in FIG. 3 demonstrated that replacing a PS internucleotide linkage with an iMeMOP in an SGLT2 ASO fully maintained the mRNA KD activity as compared to the activity of the full PS benchmark ASO. The benchmark ASO (ASO) and the PS control (ASO12) showed similar knockdown activity with an ED₅₀ of <0.5 mpk. The PO control (ASO14) lost most of the knockdown activity (ED₅₀ >0.5 mpk). The iMOP (ASO13) maintained some knockdown activity (ED₅₀ ˜0.5 mpk), which is similar to the result shown in FIG. 2.

### Example 10. Effect of iMOP Linkage at 5′-End of Antisense Strand in a GalXC Molecule

In FIG. 4, GalXC1 is a control GalXC molecule having one of the PS linkages between nucleotide 1 and 2 at the 5′-end of the antisense strand. GalXC2 is a GalXC molecule replacing the 5′-end PS linkage of the antisense strand with an iMOP linkage. The rest of the molecule are identical to the control.

Test nucleic acids were dissolved in phosphate buffered saline (PBS) and subcutaneously injected into female CD-1 mice at 0.5 mg/kg. Tissue samples were harvested 7 days after PBS or test nucleic acid injection. Tissue samples were then homogenized in QIAzol Lysis Reagent using TissueLyser II (Qiagen, Valencia, Calif.). RNA was then purified using MagMAX Technology according to manufacturer instructions (ThermoFisher Scientific, Waltham, Mass.). High capacity cDNA reverse transcription kit (ThermoFisher Scientific, Waltham, Mass.) was used to prepare cDNA. Mouse-specific ALDH2 primers (Integrated DNA Technology, Coralville, Iowa) were used for PCR on a CFX384 Real-Time PCR Detection System (Bio-Rad Laboratories, Inc., Hercules, Calif.).

The results in FIG. 4 demonstrated that replacing a PS internucleotide linkage in GalXC1 with a 4′-O-methylene phosphonate internucleotide linkage in GalXC2 derived from nucleic acid I-9 in a GalXC molecule at 5′-end of the antisense strand maintains mRNA KD activity in vivo.

### Example 11. Replacing Phosphorothioate Linkage with iMOPs on the GAP 2 Position of the SGLT2 ASO Backbone

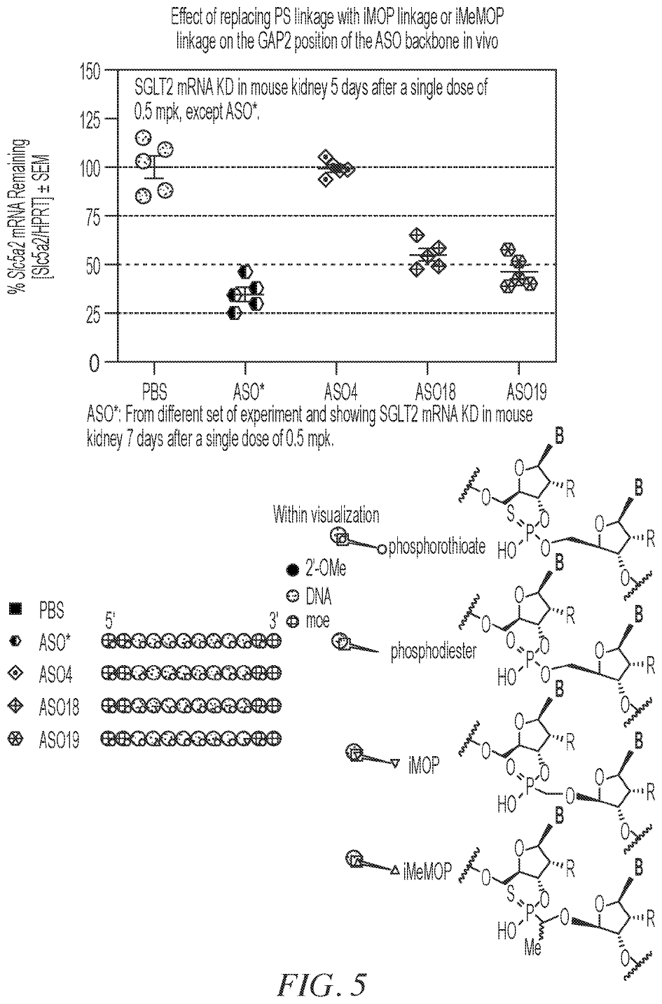

In FIG. 5, ASO is SGLT2 benchmark ASO. ASO4 is an experimental PO control that the only difference from the benchmark is the linkage between nucleotide 4 and 5 (counting from 5′-end) being internucleotide phosphodiester (PO) instead of phosphorothioate (PS) linkage. ASO18 is a test article of which the linkage between nucleotide 4 and 5 is iMOP (shown in nucleic acid I-12) instead of PS. ASO19 is the iMeMOP test article of which the linkage between nucleotide 4 and 5 is iMeMOP (shown in nucleic acid I-15) instead of PS.

The test articles described above were dissolved in phosphate buffered saline (PBS) and subcutaneously injected into female CD-1 mice at 0.5 mg/kg. Tissue samples were harvested 5 days after PBS or test article injection. (Except test article ASO* group, whose samples were harvested 7 days after injection in another experiment.) Tissue samples were then homogenized in QIAzol Lysis Reagent using TissueLyser II (Qiagen, Valencia, Calif.). RNA was then purified using MagMAX Technology according to manufacturer instructions (ThermoFisher Scientific, Waltham, Mass.). High-capacity cDNA reverse transcription kit (ThermoFisher Scientific, Waltham, Mass.) was used to prepare cDNA. Mouse-specific SGLT2 primers (Integrated DNA Technology, Coralville, Iowa) were used for PCR on a CFX384 Real-Time PCR Detection System (Bio-Rad Laboratories, Inc., Hercules, Calif.).

The results in FIG. 5 demonstrated that replacing a PS internucleotide linkage with an iMOP (ASO18) or iMeMOP (ASO19) in an SGLT2 ASO substantially maintained the in vivo mRNA KD activity as compared to the activity of the full PS benchmark ASO. ED₅₀ of both ASOs are ˜0.5 mpk. The benchmark ASO (ASO) showed knockdown activity with an ED50 of <0.5 mpk. The PO control (ASO4) lost most of the knockdown activity (ED50 >0.5 mpk).

### Example 12. Tritosome Stability

To assess the metabolic stability of the compounds with the internucleotide modification in vitro, ˜4 μM of test compounds and their corresponding control compounds were incubated in rat liver tritosomes (acid phosphatase activity) (Sekisui Xenotech, Kansas City, Kans.) at 38° C. The rat liver tritosomes are lysosomes from rat liver cells that have been treated with Triton WR 1339 (also called Tyloxapol). The incubated test compounds and their respective control compounds were collected from incubating tritosomes at different scheduled time points and subsequently extracted from the lysosomal matrix using 96-well/100 mg CLARITY® OTX™ cartridge SPE plates (Phenomenex, Torrance, Calif.) and a 96-well plate vacuum manifold per manufacturer's instructions. The eluents were evaporated using a TURBOVAP® (Biotage, Charlotte, N.C.) solvent evaporation unit and reconstituted in water and analyzed via LC-MS.

An ACQUITY UPLC® instrument (Waters Corporation, Milford, Mass.) was used to deliver mobile phases containing buffer additives at 0.4 mL/min with chromatographic separation accomplished using an ACQUITY UPLC® Oligonucleotide BEH C18 Column 1.7 μm particle sized reversed phase Ultra-Performance Liquid Chromatography (2.1×50 mm) column (Waters Corporation, Milford, Mass.). The column temperature was maintained at 70° C. and the sample injection volume was 8 μL. A SYNAPT® G2S high-resolution time-of-flight mass spectrometer (HRMS, Waters Corporation, Milford, Mass.) operating under negative ion mode and electrospray ionization (ESI) conditions was used to detect the controls, test compounds, and metabolites thereof. Zero charge-state molecular ion masses were obtained via charge-state deconvolution using PROMASS DECONVOLUTION™ software (Novatia, Newtown, Pa.). The controls, test compounds, and their metabolites were identified by comparison of experimentally determined masses to expected theoretical molecular weights.

To assess the metabolic stability of the iMOP linkage in the context of GalXC molecules, two compounds shown in FIG. 4 were tested in the tritosome assay described above. GalXC1 is a GalXC molecule with a PS linkage between nucleotide 1 and 2 at the 5′-end of the antisense strand. GalXC2 is a GalXC molecule replacing the 5′-end PS linkage of the antisense strand with an iMOP linkage.

During the 24 hours incubation period, no cleavage product was observed on the iMOP linkage of the test nucleic acid. The data in Table 2 suggests that the antisense strand containing the iMOP internucleotides linkage showed improved overall metabolic stability as compared to the control antisense strand with a PS linkage. The major metabolites observed were from the 3′-terminus of the antisense strands (data not shown).

TABLE 2
Full Length Antisense Strand Percent Remaining
after Incubation with Rat Tritosome

| Test Article | 0 h | 1 h | 2 h | 4 h | 8 h | 24 h |
|---|---|---|---|---|---|---|
| GalXC1 | 100 | 102 | 104 | 85 | 56 | 25 |
| GalXC2 | 100 | 88 | 86 | 82 | 73 | 49 |

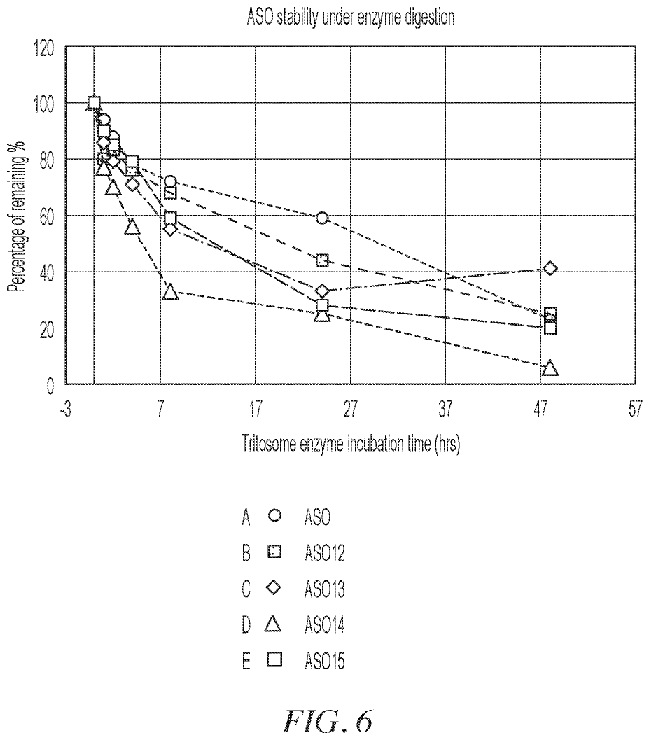

To assess the metabolic stability of the iMOP and iMeMOP linkage in the context of ASO platform, test articles shown in FIG. 3 were tested in the tritosome assay described above. As shown in Table 3, the test articles with iMOP or iMeMOP linkage showed similar metabolic stability as compared to the parent control and the 2′OMe PS control. The PO control of which the linkage between nucleotide 10 and 11 is a phosphodiester linkage (ASO14) showed the least stability. FIG. 6 shows the results of Table 3 in graphical form.

TABLE 3
Full Length Antisense Strand Percent Remaining
after Incubation with Rat Tritosome

| Test Article | 0 h | 1 h | 2 h | 4 h | 8 h | 24 h | 48 h |
|---|---|---|---|---|---|---|---|
| ASO | 100 | 94 | 88 | 78 | 72 | 59 | 23 |
| ASO12 | 100 | 80 | 83 | 76 | 68 | 44 | 25 |
| ASO13 | 100 | 86 | 79 | 71 | 55 | 33 | 41 |
| ASO14 | 100 | 77 | 70 | 56 | 33 | 25 | 6 |
| ASO15 | 100 | 90 | 85 | 79 | 69 | 28 | 20 |

### Example 13. Effect of iMOP and iMeMOP Modifications on Duplex Stability

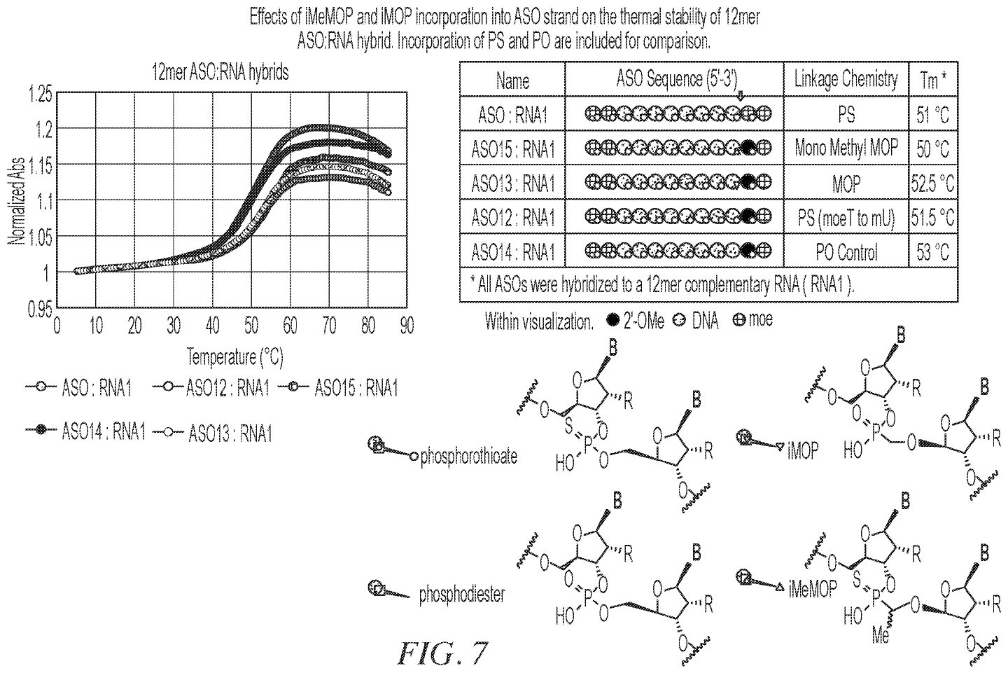

Duplex formation and melting of SGLT2 ASOs and RNA1, a 12mer RNA designed to bind to the SGLT2 ASOs with full Watson-crick complementarity, was monitored by ultraviolet (UV) spectroscopy and on an Agilent Cary 3500 UV-VIS spectrophotometer equipped with a Peltier temperature controller. Duplex concentration was 2 μM (4 μM total concentration of strands) in PBS (Phosphate Buffered Saline) (1×, pH 7.4). After heating to 90° C., samples were slowly cooled to room temperature and refrigerated overnight. Samples were then transferred into cold cuvettes in the spectrophotometer and the change in absorbance at 260 nm was monitored upon heating from 5° C. to 90° C. at a rate of 0.5° C./min. Samples were kept under flowing nitrogen when below 20° C. and absorbance values were recorded every 30 seconds. Tm values were calculated using the baseline method and shown in FIG. 7.

ASO is fully phosphorothioated SGLT2 benchmark ASO. ASO14 is the PO control of the benchmark and has a phosphodiester linkage between nucleotide 10 and 11. ASO13 is the iMOP test article in which the linkage between nucleotide 10 and 11 is iMOP instead of PS. ASO15 is the iMeMOP test article in which the linkage between nucleotide 10 and 11 is iMeMOP instead of PS. Results indicate that ASO14 exhibits the highest thermal stability when bound to complementary RNA1. Results also indicate that replacing a PS internucleotide linkage with novel iMeMOP or iMOP modifications maintain the ASO:RNA duplex thermal stability while incorporation of iMeMOP is marginally destabilizing by −1° C. (see ASO15:RNA1 in FIG. 6), incorporation of iMOP is stabilizing by +1.5° C. (see ASO13:RNA1 in FIG. 7).

### Example 14. Effect of iMOP and iMeMOP Modifications on RNase H Activity

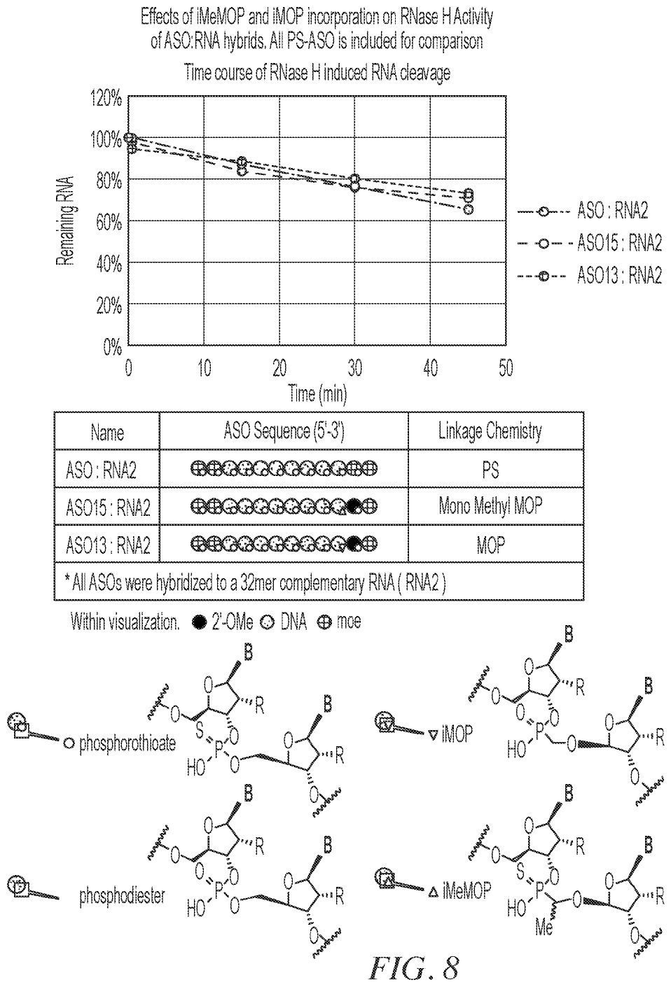

It is known that RNase H enzyme digests the RNA portion of an ASO:RNA hybrid while the ASO strand remains untouched. In order to monitor the efficacy of internucleotide linkage modifications in eliciting RNase H activity when incorporated to ASOs, the iMOP and iMeMOP modified ASOs were hybridized to a complementary RNA and tested for their susceptibility to cleavage by human RNase H. Cleavage reactions were monitored using high-resolution LC-MS method instead of classical electrophoretic methods. Analysis of mass peaks of generated RNA fragments enables the determination of the exact cleavage sites on RNA while quantification of peak areas corresponding to RNA fragments and/or remaining full-length RNA on the LC spectra allows comparison of the cleavage reaction kinetics induced by different ASOs (FIG. 8).

A 32-nucleotide long RNA strand (RNA2) was designed containing a 12-nucleotide stretch with full complementarity to SGLT2 12mer ASOs. Annealing of each ASO to the complement RNA provides the duplexed substrates (ASO15:RNA2, ASO13:RNA2, and ASO:RNA2). ASO is the SGLT2 benchmark ASO in which all linkages are PS. ASO13 is the iMOP test article in which the linkage between nucleotide 10 and 11 is iMOP instead of PS. ASO15 is the iMeMOP test article in which the linkage between nucleotide 10 and 11 is iMeMOP instead of PS.

Generally, 2 nmol of each antisense oligonucleotide was mixed with 1 nmol of RNA in 1× RNase H reaction buffer (50 mM Tris-HCl, 75 mM KCl, 3 mM MgCl₂, and 10 mM DTT at pH 8.3). Samples were heated at 90° C. for 5 minutes and slowly cooled to room temperature to allow the duplex substrates to form. Each annealing solution was made of a 2-fold excess of AON relative to the RNA to ensure all RNA is hybridized to ASO and free RNA does not exist in solution. Next, 100 μL aliquots were transferred into glass total recovery MS vials and kept at LC-MS sample holder at 20° C. (assay temperature) for 1 minute. The assay temperature was chosen to be much lower than the thermal melting temperatures of the ASO:RNA hybrids to further ensure all RNA is hybridized to ASO. After 1-minute incubation, ASO:RNA duplexed substrates were analyzed on a Waters Synapt high resolution LC-MS yielding the spectra for 0 timepoint.

RNA cleavage reactions were then initiated by addition of 2 μL of 0.25 U freshly diluted E. coli RNase H enzyme in 1× RNase H buffer. The enzyme was handled over ice to avoid any loss of activity. The mixture was gently mixed by pipetting and the RNA cleavage was monitored on LC-MS at 30 sec, 15 min, 30 min and 45 min timepoints post enzyme addition. The 0.25 U optimal RNase H concentration for these assays was chosen from a series of preliminary enzyme dilutions (10 U, 5 U, 1 U, 0.5 U and 0.25 U). At 0.25 U, the digestion of RNA is slow allowing the calculation and comparison of cleavage rates as shown in FIG. 8. At each timepoint, the fraction of RNA converted to cleavage product is calculated through quantification of LC peak area corresponding to remaining full-length RNA at that timepoint. As shown in FIG. 8, results indicate that replacing a PS internucleotide linkage with iMeMOP or iMOP in an SGLT2 ASO sequence fully maintains the RNase H activity with comparable cleavage rates to that of the SGLT2 benchmark.

While a number of embodiments of this invention have been described herein, it is apparent that the basic examples provided herein may be altered to provide other embodiments that utilize the nucleic acid or analogues thereof and methods of this invention. Therefore, it will be appreciated that the scope of this invention is to be defined by the specification and appended claims rather than by the specific embodiments that have been represented by way of example.

We claim:

1. A nucleic acid or analogue thereof comprising a 4′-O-methylene phosphonate internucleotide linkage, wherein the 4′-O-methylene phosphonate internucleotide linkage is represented by formula I: 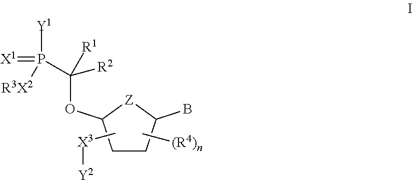
or a pharmaceutically acceptable salt thereof, wherein:
B is a nucleobase or hydrogen;
R¹ and R² are independently hydrogen, halogen, R⁵, —CN, —S(O)R, —S(O)₂R, —Si(OR)₂R, —Si(OR)R₂, or —SiR₃, or:
R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X¹ is O, S, or NR;
X² is —O—, —S—, —B(H)₂—, or a covalent bond;
X³ is —O—, —S—, —Se—, or —N(R)—;
Y¹ is a linking group attaching to the 2′- or 3′-terminal of a nucleoside, a nucleotide, or an oligonucleotide;
Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
Z is —O—, —S—, —N(R)—, or —C(R)₂—; and
n is 0, 1, 2, 3, 4, or 5.

2. The nucleic acid or analogue thereof according to claim 1, wherein the 4′-O-methylene phosphonate internucleotide linkage is selected from any one of the representative formulae: 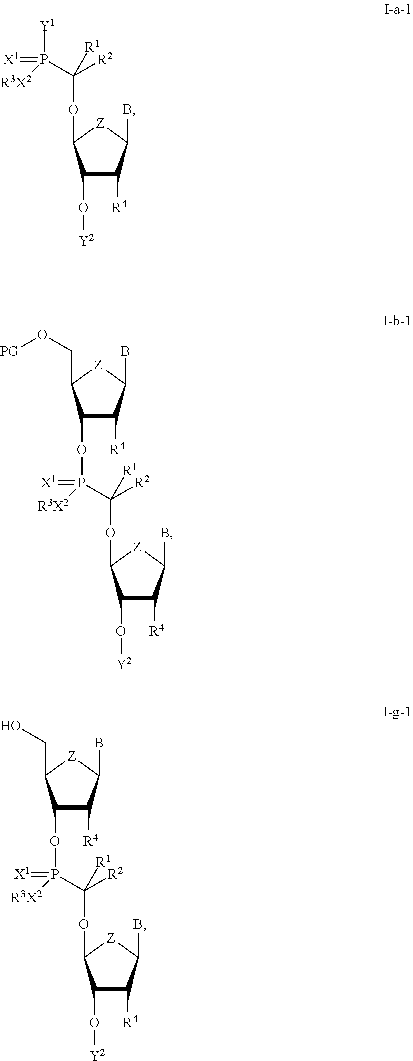 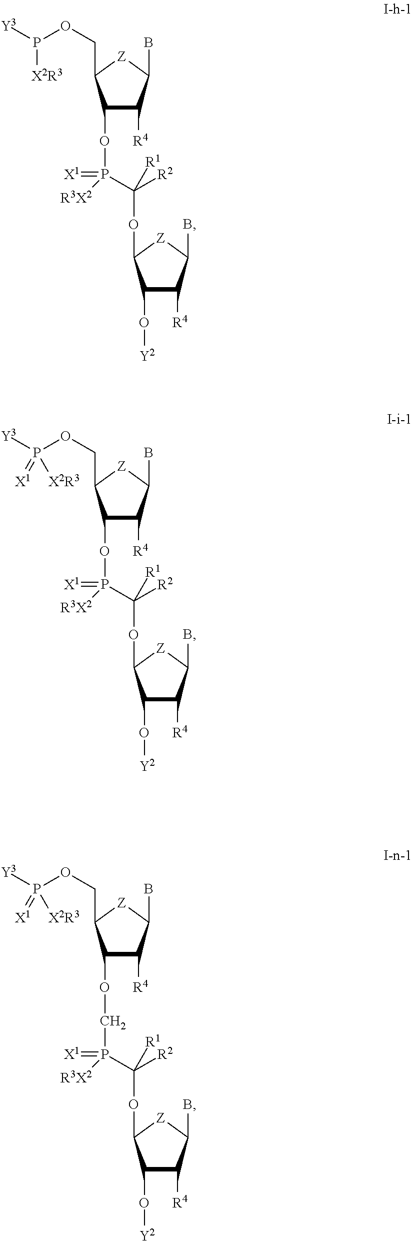 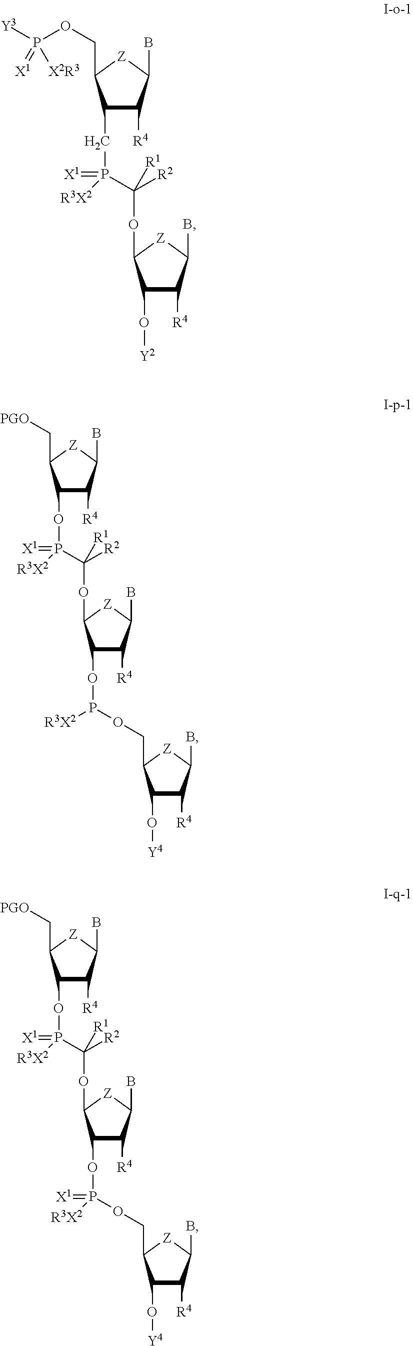
or a pharmaceutically acceptable salt thereof, wherein:
Y³ is a linking group attaching to the 2′- or 3′-terminal of a nucleoside, a nucleotide, or an oligonucleotide; and
Y⁴ is hydrogen, a protecting group, a phosphoramidite analogue, an internucleotide linking group attaching to the 4′- or 5′-terminal of a nucleoside, a nucleotide, or an oligonucleotide, or a linking group attaching to a solid support.

3. The nucleic acid or analogue thereof according to either claim 1 or claim 2, wherein the nucleic acid or analogue thereof is selected from any one of the following formulae: 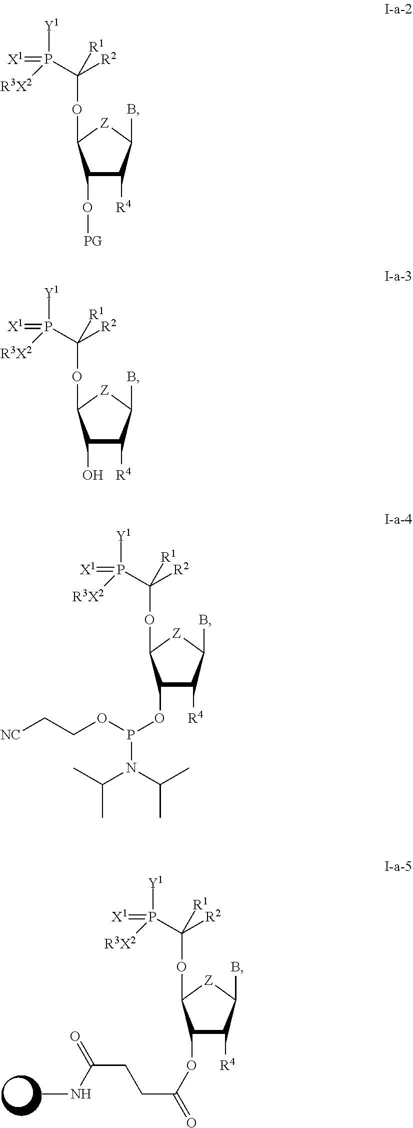 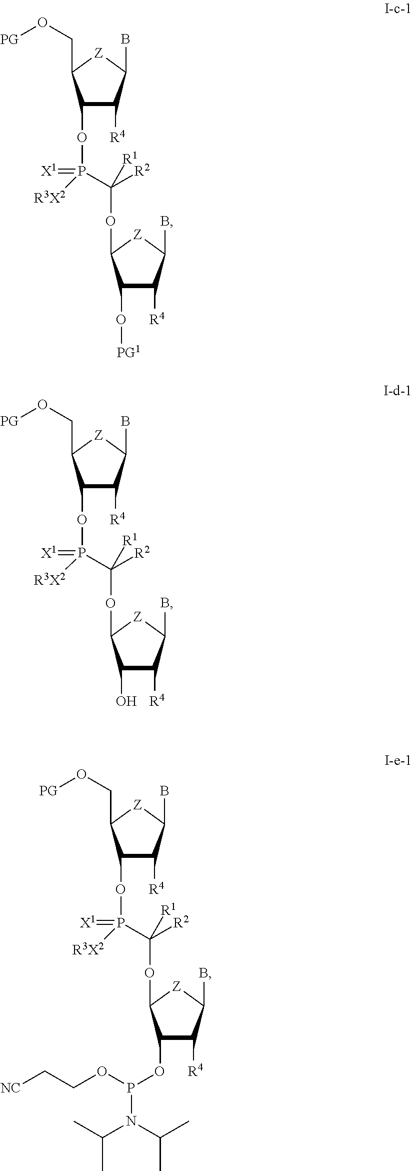 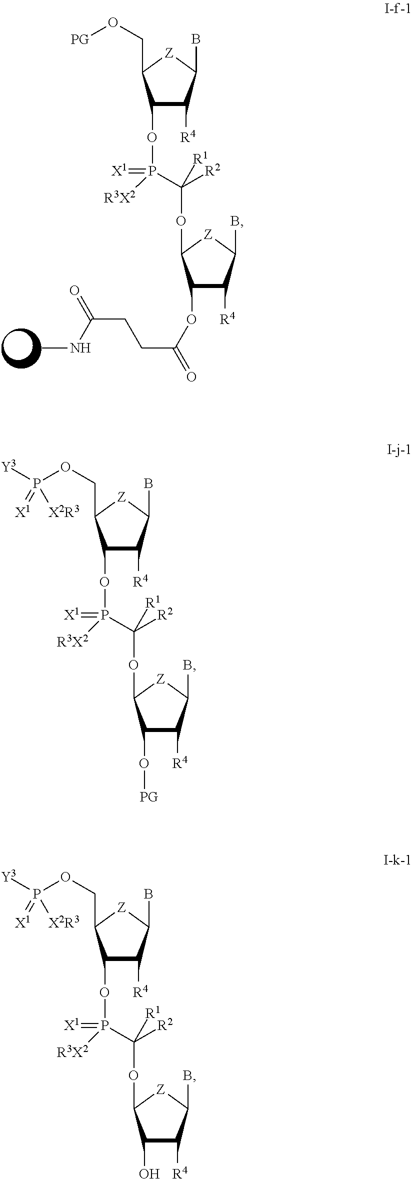 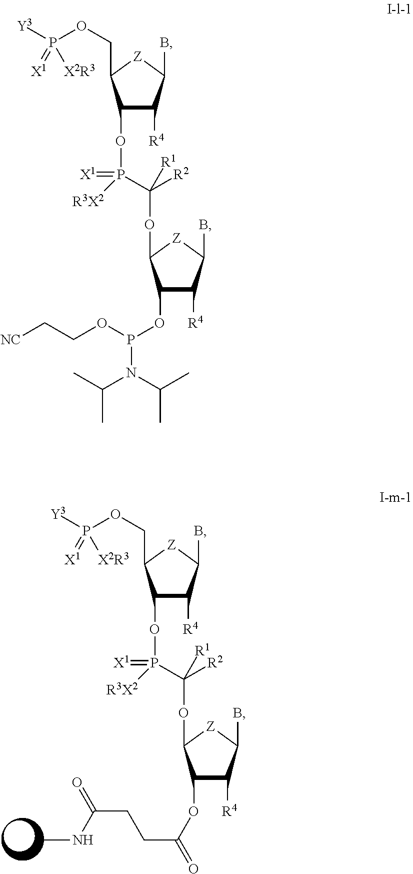
or a pharmaceutically acceptable salt thereof.

4. The nucleic acid or analogue thereof according to any one of claims 1-3, wherein R¹ is hydrogen and R² is hydrogen or methyl.

5. The nucleic acid or analogue thereof according to any one of claims 1-4, wherein each R⁴ is independently hydrogen, hydroxy, fluoro, methoxy, or

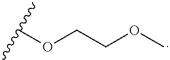 [SMILES: *OCCOC]

6. The nucleic acid or analogue thereof according to any one of claims 1-5, wherein each B is selected from

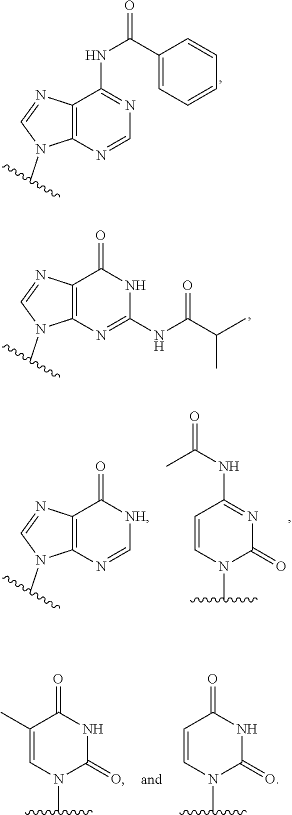

7. The nucleic acid or analogue thereof according to any one of claims 1-6, wherein said nucleic acid or analogue thereof is selected from any one of those depicted in Table 1, or a pharmaceutically acceptable salt thereof.

8. The nucleic acid or analogue thereof according to claim 1, wherein the nucleic acid or analogue thereof is a double-stranded RNAi inhibitor molecule comprising a first strand and a second strand, wherein the first strand is a sense strand and the second strand is an antisense strand.

9. The nucleic acid or analogue thereof according to claim 8, wherein the double stranded RNAi inhibitor molecule comprises a region of complementarity between the sense strand and the antisense strand of 15 to 45 nucleotides.

10. The nucleic acid or analogue thereof according to claim 9, wherein the region of complementarity between the sense strand and the antisense strand is 20 to 30 nucleotides.

11. The nucleic acid or analogue thereof according to claim 10, wherein the region of complementarity between the sense strand and the antisense strand is 21 to 26 nucleotides.

12. The nucleic acid or analogue thereof according to claim 9, wherein the region of complementarity between the sense strand and the antisense strand is 19 to 24 nucleotides.

13. The nucleic acid or analogue thereof according to claim 12, wherein the region of complementarity between the sense strand and the antisense strand is 19 to 21 nucleotides.

14. The nucleic acid or analogue thereof according to claim 8, wherein the double-stranded RNAi inhibitor molecule contains a tetraloop.

15. The nucleic acid or analogue thereof according to claim 1, wherein the nucleic acid or analogue thereof is a single stranded nucleic acid.

16. The nucleic acid or analogue thereof according to claim 15, wherein the single stranded nucleic acid is a single stranded RNAi inhibitor molecule.

17. The nucleic acid or analogue thereof according to claim 15, wherein the single-stranded nucleic acid is a conventional antisense nucleic acid, a ribozyme or an aptamer.

18. The nucleic acid or analogue thereof according to either claim 16 or claim 17, wherein the single stranded RNAi inhibitor molecule is 14-50 nucleotides in length.

19. The nucleic acid or analogue thereof according to claim 18, wherein the single stranded RNAi inhibitor molecule is about 16-30, 18-22, or 20-22 nucleotides in length.

20. The nucleic acid or analogue thereof according to claim 1, wherein the nucleic acid or analogue thereof is a naked nucleic acid.

21. The nucleic acid or analogue thereof according to claim 1, further comprising at least one delivery agent, wherein the at least one delivery agent is conjugated to the nucleic acid or analogue thereof to facilitate transport of the nucleic acid or analogue thereof across an outer membrane of a cell.

22. The nucleic acid or analogue thereof according to claim 1, wherein the delivery agent is selected from the group consisting of carbohydrates, peptides, lipids, vitamins and antibodies.

23. The nucleic acid or analogue thereof according to claim 1, wherein the delivery agent is selected from N-Acetylgalactosamine (GalNAc), mannose-6-phosphate, galactose, oligosaccharide, polysaccharide, cholesterol, polyethylene glycol, folate, vitamin A, vitamin E, lithocholic acid and a cationic lipid.

24. A pharmaceutical composition comprising a nucleic acid or analogue thereof according to claim 1, and a pharmaceutically acceptable carrier, adjuvant, or vehicle.

25. A method for reducing expression of a target gene in a subject in need thereof, comprising administering the pharmaceutical composition of claim 24 to the subject in an amount sufficient to reduce expression of the target gene.

26. A method for treating cancer, a viral infection, or genetic disorder in a subject in need thereof, comprising administering the pharmaceutical composition of claim 24 to the subject in an amount sufficient to treat the cancer, viral infection, or genetic disorder.

27. The method according to either claim 24 or claim 25, wherein the administering comprises systemic administration.

28. A method for preparing a nucleic acid or analogue thereof comprising a 4′-O-methylene phosphonate internucleotide linkage, wherein the 4′-O-methylene phosphonate internucleotide linkage is represented by formula I-c: 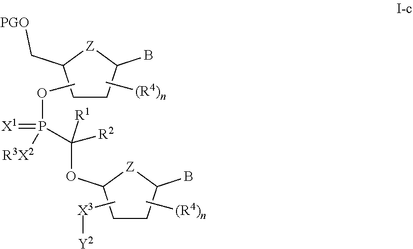
or a pharmaceutically acceptable salt thereof, comprising the steps:
(a) providing a nucleic acid or analogue thereof of formula A4: 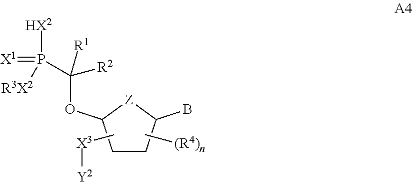
or pharmaceutically acceptable salt thereof, and
(b) condensing the nucleic acid or analogue thereof of formula A4 with a nucleoside or analogue thereof of formula A5: 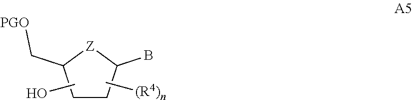
to form the nucleic acid or analogue thereof comprising formula I-b, wherein:
each B is a nucleobase or hydrogen;
PG is a suitable hydroxyl protecting group;
R¹ and R² are independently hydrogen, halogen, R⁵, —CN, —S(O)R, —S(O)₂R, —Si(OR)₂R, —Si(OR)R₂, or —SiR₃, or:
R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X¹ is O, S, or NR;
each X² is independently —O—, —S—, —B(H)₂—, or a covalent bond;
X³ is —O—, —S—, —Se—, or —N(R)—;
Y² is hydrogen, a protecting group, a phosphoramidite analogue, an internucleotide linking group attaching to the 4′- or 5′-terminal of a nucleoside, a nucleotide, or an oligonucleotide, or a linking group attaching to a solid support;
each Z is independently —O—, —S—, —N(R)—, or —C(R)₂—; and
each n is independently 0, 1, 2, 3, 4, or 5.

29. The method of claim 27, wherein Y² is a protecting group.

30. The method of claim 29, further comprising the steps of preparing a nucleic acid of formula I-d: 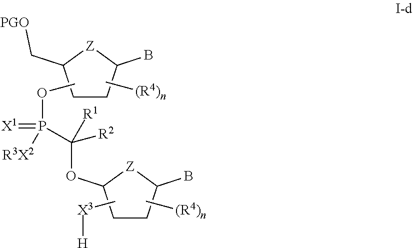
or a pharmaceutically acceptable salt thereof, comprising the steps:
(a) providing a nucleic acid or analogue thereof comprising of formula I-c: 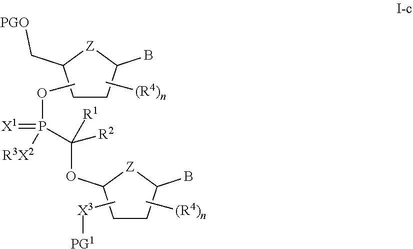
or pharmaceutically acceptable salt thereof, and
(b) deprotecting the nucleic acid or analogue thereof comprising formula I-c to form the nucleic acid or analogue thereof comprising formula I-d, wherein:
each B is a nucleobase or hydrogen;
PG is a suitable hydroxyl protecting group;
PG¹ is a protecting group;
R¹ and R² are independently hydrogen, halogen, R⁵, —CN, —S(O)R, —S(O)₂R, —Si(OR)₂R, —Si(OR)R₂, or —SiR₃, or:
R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X¹ is O, S, or NR;
each X² is independently —O—, —S—, —B(H)₂—, or a covalent bond;
X³ is —O—, —S—, —Se—, or —N(R)—;
each Z is independently —O—, —S—, —N(R)—, or —C(R)₂—; and
each n is independently 0, 1, 2, 3, 4, or 5.

31. The method of claim 30, further comprising the steps of preparing a nucleic acid or analogue thereof of formula I-e: 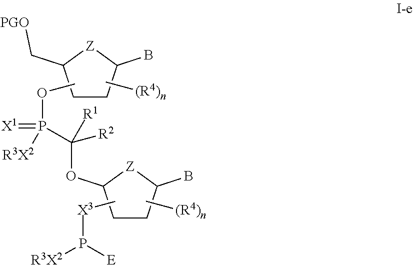
or a pharmaceutical acceptable salt thereof, comprising the steps:
(a) providing a nucleic acid or analogue thereof of formula I-d: 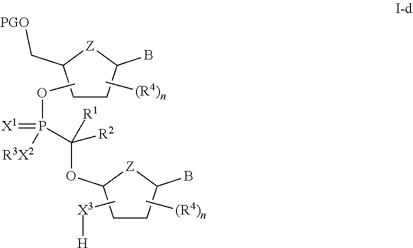
(b) reacting the nucleic acid or analogue thereof of formula I-d with a P(III) forming reagent to form the nucleic acid or analogue thereof of formula I-e, wherein:
each B is a nucleobase or hydrogen;
PG is a suitable hydroxyl protecting group;
R¹ and R² are independently hydrogen, halogen, R⁵, —CN, —S(O)R, —S(O)₂R, —Si(OR)₂R, —Si(OR)R₂, or —SiR₃, or:
R¹ and R² on the same carbon are taken together with their intervening atoms to form a 3-7 membered saturated or partially unsaturated ring having 0-3 heteroatoms, independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, a suitable prodrug, R⁵, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃;
each R⁵ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
E is a halogen or —NR₂;
X¹ is O, S, or NR;
each X² is independently —O—, —S—, —B(H)₂—, or a covalent bond;
X³ is —O—, —S—, —Se—, or —N(R)—;
each Z is independently —O—, —S—, —N(R)—, or —C(R)₂—; and
each n is independently 0, 1, 2, 3, 4, or 5.

32. The method according to any one of claims 28-31, wherein each R¹ is hydrogen and R² is hydrogen of methyl.

33. The method according to any one of claims 28-32, wherein each R⁴ is independently hydrogen, hydroxy, fluoro, methoxy, or

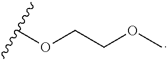 [SMILES: *OCCOC]

34. The method according to any one of claims 28-33, wherein each B is selected from [chemical structure]
